SEQUENCE LISTING (112 sequences)

SEQ ID NO: 1 - protein, 289 aa, Escherichia coli K12
MSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 2 - protein, 388 aa, Escherichia coli K12
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 3 - DNA, 2036 bp, Escherichia coli K12
atgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 4 - protein, 295 aa, Azotobacter vinelandii DJ
MSILVNKDTKVICQGFTGSQGTFHSEQAIAYGTRMVGGVTPGKGGLVHLDLPVFDTVREAVEATGADASVIYVPAPFCKDSILEAAFAGVRLIVCITEGVPTLDMLQVKLKCDELGVRLIGPNCPGVITPGECKIGIQPGNIHMPGRVGIVSRSGTLTYEAVKQTTDAGFGQSTCVGIGGDPIPGSSFIDILGLFQDDPQTEAIVMIGEIGGSAEEEAAAYIKAKVDKPVVSYIAGVTAPSGKRMGHAGAIISGGKGTADEKFAALQDAGVQTVRSLADIGKALAELTGWERKQS

SEQ ID NO: 5 - protein, 389 aa, Azotobacter vinelandii DJ
MNLHEYQGKQLFAEYGLPVSRGVAIDTPEAAAEACDRIGGDCWVAKVQVHAGGRGKAGGVKLVKSREEAKVFAVNWLGKRLVTYQTDASGQPVGKILVEACTEIERELYLGAVVDRSSRRIVFMASTEGGVNIEQVAHETPEKILKASIDPLVGAQPFQARDLAFRLGLEGDQLKQFTHIFIGLAKLFQEHDLALVEVNPLVVQKDGNLLCLDAKINLDTNALFRQPRLRAMHDPSQDDPREVHAAKWELNYVALEGNIGCMVNGAGLAMGTMDIVNLHGGRPANFLDVGGGATKERVTEAFKIILSDAKVKAVLVNIFGGIVRCDMIAEGIIGAVREVGVKVPVVVRLEGNNAELGAEMLARSGLNIIPASTLTDAAVQVVKAAEDNP

SEQ ID NO: 6 - DNA, 2054 bp, Azotobacter vinelandii DJ
atgaatctccatgaatatcagggcaagcagcttttcgccgaatatggtttacccgtgtcccgaggcgttgccatcgataccccggaggccgcggcggaggcctgcgacaggattggcggcgactgctgggtcgcgaaggtccaggtgcatgccggcggtcgtggcaaggccggtggcgtcaagctggtcaagagccgggaggaggcgaaggtcttcgccgtcaactggctgggcaagcgactggtgacctaccagaccgacgcttcggggcagccggtcggcaagatcctggtcgaggcctgcaccgagatcgagcgggagctttacctgggagcggtggtcgatcgctcgagccgccgcatcgtcttcatggcctcgaccgagggcggggtgaacatcgagcaggtcgcccatgaaacgcccgagaagatcctcaaggccagcatcgaccccctggtcggcgcccagccgttccaggcccgcgacctggccttccggctgggtctcgaaggcgatcagctcaagcagttcacccatatcttcatcggtctggccaagctgttccaggagcacgatctggccctggtggaggtgaatccgctggtggtccagaaggacggcaatctgctctgcctggacgccaagatcaatctcgataccaacgccctgttccgccaacccagactgcgcgccatgcacgacccttcccaggacgatccccgcgaagtgcatgcggcgaagtgggagctgaactacgtggccctcgagggcaacatcggctgcatggtcaacggcgccggactggccatgggcaccatggacatcgtcaatctccatgggggccggccggccaacttcctcgacgtcggcggcggcgcgaccaaggagcgggtgaccgaggccttcaagatcattctctccgatgccaaggtaaaagccgtgctggtcaacatcttcggcggcatcgtgcgctgcgacatgatcgccgaaggcatcatcggcgcggtccgggaggtaggcgtcaaggttccggtggtggtccgcctggagggcaacaacgcggaactgggcgccgagatgctggcccggagcggcctgaacatcattccggccagcaccctgaccgatgcggcggtgcaggtggtcaaggcagcggaggacaacccatgagtattttggtcaacaaggacaccaaggtcatctgccagggattcaccggtagccaggggaccttccacagcgaacaggccattgcctatggcacccggatggtcggaggcgtgacgccgggcaagggaggactcgtccatctcgacctgccggtattcgacacggtccgcgaggccgtggaggccaccggcgccgacgcctcggtcatctacgtacccgcgcccttctgcaaggattccattctcgaggcggctttcgccggtgtccggctgatcgtctgcatcaccgagggcgtaccgaccctcgacatgctgcaggtcaagctcaagtgcgacgagctgggcgtgcgcctgatcggccccaactgtccgggcgtgatcactcccggcgagtgcaagatcggcatccagccgggcaatatccacatgccgggcagggtcggcatcgtttcccggtcgggcaccctgacttacgaggcggtgaagcagaccaccgacgcgggcttcggccagtccacctgcgtgggtatcggtggcgacccgattccggggtccagtttcatcgatatcctcggtctgttccaggacgatccgcagaccgaagccatcgtgatgatcggcgaaatcggcggcagtgccgaggaggaggcggcggcctacatcaaggccaaggtcgacaagccggtggtttcctacatcgccggcgtcaccgcgccctcgggcaagcgcatggggcatgccggtgcgatcatctccggcggcaagggcactgcggacgagaagttcgccgccctgcaggatgccggcgtgcagaccgtgcgttccctggcggatatcggcaaggccctggccgaactgaccggctgggagaggaagcagtcctga

SEQ ID NO: 7 - protein, 294 aa, Bradyrhizobium sp.BTAi1
MSILIDKNTKVICQGFTGKNGTFHSEAAIAYGTKMVGGTSPGKGGSTHLGLPVFDTVKEAREATGADASVIYVPPPGAADAICEAIDAEVPLIVCITEGIPVLDMVRVKRSLQGSKSRLIGPNCPGVMTAGECKIGIMPANIFKPGSVGIVSRSGTLTYEAVFQTTSEGLGQTTAVGIGGDPVKGTEFIDMLEMFLADPKTESIIMIGEIGGSAEEDAAQFIKDEAKRGRKKPMVGFIAGVTAPPGRRMGHAGAIISGGKGDAGSKTAAMEAAGITVSPSPARLGKTLVEKLKS

SEQ ID NO: 8 - protein, 398 aa, Bradyrhizobium sp.BTAi1
MNIHEYQAKALLHEFGVPISKGVPVLRPEDSDAAAKALGGPVWVVKSQIHAGGRGKGKFKEASAGDKGGVRLAKSIDEVNAFAKQMLGATLVTVQTGPDGKQVNRLYIEDGSDIDKEFYLSLLVDRETSKVAFVVSTEGGVNIEDVAHSTPEKIITFSVDPATGVMPHHGRAVAKALKLSGDLAKQAEKLTIQLYTAFVAKDMAMLEINPLVVTKQGQLRVLDAKVSFDSNALFKHPEVVALRDETEEDAKEIEASKYDLNYVALDGTIGCMVNGAGLAMATMDIIKLYGMEPANFLDVGGGASKEKVAAAFKIITADPNVKGILVNIFGGIMKCDVIAEGVVAAVKEVGLKVPLVVRLEGTNVDLGKKIISESGLNVLPADNLDDAAQKIVKAVKGG

SEQ ID NO: 9 - DNA, 2138 bp, Bradyrhizobium sp.BTAi1
atgaacattcacgaatatcaggccaaggcactgctgcacgagttcggcgtgccgatttccaagggcgtgccggtgctccgtccggaggactcggatgcggcggcgaaggcgctcggcggtccggtctgggtcgtgaagagccagatccacgccggcggccgtggcaagggcaagttcaaggaggcctcggccggcgacaagggcggcgtccgcctcgccaagtcgattgacgaggtcaatgcgttcgccaagcagatgctcggcgcaaccctcgtcaccgtgcagaccggccccgatggcaagcaggtcaaccgcctctacatcgaggacggctcggatatcgacaaggaattctacctgtcgctgctggtcgatcgcgagacctcgaaggtcgctttcgtggtgtcgaccgaaggcggcgtcaacatcgaggacgttgctcacagcacgcctgagaagatcatcaccttctcagtcgatccggccaccggcgtgatgccgcatcacggtcgcgccgtcgccaaggcgctgaagctctcgggcgatctcgccaagcaggccgagaagctgaccatccagctctataccgccttcgtcgccaaggacatggcgatgctcgagatcaacccgctggtcgtcaccaagcagggccagctgcgtgtgctcgacgccaaggtgtcgttcgactccaacgcgctgttcaagcaccccgaggtcgtggcgctgcgtgacgagaccgaggaagacgccaaggagatcgaggcctccaaatacgatctcaactatgtcgcgctcgacggcaccatcggctgcatggtcaacggcgccggcctcgcgatggcgacgatggacatcatcaagctctacggcatggagccggccaacttcctcgacgtcggcggcggcgccagcaaggagaaggtcgcggcggcgttcaagatcatcaccgccgacccgaacgtgaagggcatcctggtcaacatcttcggcggcatcatgaagtgcgatgtcatcgccgagggcgtcgtggccgcggtcaaggaagtcggcctgaaggtgccgctggtggtgcgcctcgaaggcaccaatgtcgatctcggcaagaagatcatcagcgagtccggtctgaacgtgctgcccgccgacaatctcgacgacgccgcgcagaagatcgtcaaggccgtcaagggaggctgagcgccgtttcaggcgctcgcttagctcctcaccgcaacgcttttagagaaagcacgatgtccattctcatcgacaagaacaccaaggtcatctgtcagggcttcactggcaagaacggcaccttccactccgaggcggcgatcgcctacggcaccaagatggtcggcggcacctcgccgggcaaaggcggctcgacccatctcggcctgccggtgttcgacaccgtcaaggaggctcgcgaggccactggcgctgacgcgtcggtgatctacgtgccgccgccgggtgcggccgacgccatttgcgaggcgatcgacgccgaggtcccgctgatcgtctgcatcaccgagggcatcccggtgctcgacatggtcagggtcaagcgctcgctgcagggctccaagtcgcgcctgatcggcccgaactgcccgggcgtcatgaccgccggagagtgcaagatcggcatcatgccggccaatatcttcaagcccggctcggtcggcatcgtgtcacgctccggcacgctgacctatgaagcggtgttccagaccacctcggaaggcctcggtcagaccaccgcggtcggtatcggcggcgacccggtcaagggcaccgagttcatcgacatgctggagatgttccttgccgaccccaagaccgagtcgatcatcatgatcggcgagatcggcggctcggccgaggaagacgcggcccagttcatcaaggacgaggccaagcgcggccgcaagaagccgatggtcggattcatcgccggcgtcacggcgcctccgggccgtcgcatgggccatgccggcgcgatcatctcgggcggcaagggtgatgccggttcgaagacggccgcgatggaagcggctggtatcacggtgtcgccgtcgccggcgcggctcggcaaaacgcttgtcgaaaagttgaaatcctga

SEQ ID NO: 10 - protein, 291 aa, Azospirillum sp. B510
MAVLVDKNTKVICQGFTGAQGTFHSEQAIAYGTKMVGGVTPGKGGAKHLDLPIFDTVAEAVEKTGANASVIYVPPPFAADAILEAIDAEIPLVVCITEGIPVLDMVRVKRALNGSATRLIGPNCPGVITPDECKIGIMPGHIHKRGKIGIVSRSGTLTYEAVAQTTAAGLGQTTCIGIGGDPVNGTNFVDSLELFVKDPETEGIIMIGEIGGDAEVKGAEFIKASGTRKPVVGFIAGRTAPPGRRMGHAGAVISGGNDTADFKIDFMKSVGIAVADSPASLGSTMLKVFKG

SEQ ID NO: 11 - protein, 389 aa, Azospirillum sp. B510; identical to SEQ ID NO: 5

SEQ ID NO: 12 - DNA, 2074 bp, Azospirillum sp. B510
atgaacatccatgagtaccaggcgaaaagcctgctgaagaagtacggcgtcgcggttccccgcggcggcgtcgcctacaccccgcaggaggccgagacggtcgcccgcgagctgggcggtccggtctgggtggtgaagtcccagatccacgccggcggccgcggcgccggccgcttcaaggacaaccccgaaggcaagggcggcgtccgcgtcgtcaagtcgatcgaggatgtcggcaagaacgccgccgagatgctgaaccacgttctcgtgaccaagcagaccggcgccgaaggccgcgaggtcaagcgcctctatgtcgaggaaggcgccgacatcaagcgcgagctgtatctcggcatgctgatcgaccgcgccaccggccgcgtgacgatcatggcctcgaccgaaggcggcatggagatcgaggaggtcgcccacaacacgccggagaagatcatcaaggtcgcggtcgacccggccaccggcatccagggctaccacacccgcaaggtcgccttcgcgctcggcctggaaggcaagcaggtcggtgcggccgccaagttcatccaggccgcctatcaggccttcatcgacctcgactgcgccatcgtcgagatcaacccgctgatcgtcaccgggtcgggcgacatcctggcgctcgacgccaagatgaacttcgacgacaacgcgctgttccgtcacaaggacgttgaagagctgcgcgacgaggccgaagaggacccggcggagatcgaggcggccaagcacagcctcaactacgtcaagctcgatggcaacatcggctgcatggtcaacggcgccggcctggcgatggccaccatggacatcatcaagctctatggcggcgagccggccaacttcctcgacgtcggcggcggcgccaccaaggagcgcgtcaccgcggccttcaagctgatcctgtccgacagcaacgtcgaaggcatcctggtcaacatcttcggcggcatcatgcgctgcgacgtgatcgccgagggcgtggtcgccgcggcgcgcgaagtgcatctgcatgttccgctggtggtgcgcctggaaggcaccaacgtcgatctgggcaagaagatcctggccgaatccggcctgccgatcctctcggccgacaacctcgccgacgccgccgagaaggtggtcaaggccgtgaaggaggccgcgtgaaatggctgttctcgtcgataagaacacgaaggtgatctgccagggcttcaccggagcccagggcaccttccactccgagcaggccatcgcctacggcaccaagatggtcggcggcgtgacccccggcaagggcggcgccaagcatcttgacctgccgatcttcgacaccgtcgccgaggcggtcgagaagaccggggccaacgcctcggtgatctatgtgccgccgcccttcgcggccgacgcgatcctggaggcgatcgacgccgagatcccgctggtggtctgcatcaccgaaggcatcccggtgctcgacatggtccgcgtcaagcgcgccctcaacggctccgccacgcgcctgatcggcccgaactgccccggcgtcatcacgccggacgagtgcaagatcggcatcatgccgggccacatccacaagcgtggcaagatcggcatcgtctcgcgctccggcacgctgacctatgaggccgtcgcgcagaccacggcggccggtctcggccagaccacctgcatcggcatcggcggcgacccggtcaacggcaccaacttcgtcgacagcctggagctgttcgtgaaggacccggagaccgagggcatcatcatgatcggcgagatcggcggtgacgccgaggtcaagggcgcggagttcatcaaggcgtcgggcacgaggaagccggtcgtcggcttcatcgccggccgcacggcgcctccgggccgccgcatgggccatgccggtgccgtcatctccggcggcaacgacaccgccgacttcaagatcgacttcatgaagtcggtcggcatcgccgtcgccgacagccccgccagcctgggctccaccatgctgaaggtgttcaagggctga

SEQ ID NO: 13 - protein, 640 aa, Halobacterium sp. NRC-1
MPYWSTAGPDQIMTDDELIWRIAGGSGDGIDSTSQNFAKALMRSGLDVFTHRHYPSRIRGGHTYVEIRARDGTVTSRGDGYNFLLALGDSFARNPSEEAVYGDEEVKPLTENLDDLRAGGVIIYDEGLLDDEDVGDLEQQADANDWHLYPLDLRGLAKEHGREVMRNTAGVGATAALIDMDLDHIEDLMSDAMGGDILEQNLTVLRDAYEQVSEMEHTHDLSVPTGSHDEPQVLMSGSHAIAYGAIDAGCRFISGYPMTPWTDAFTIMTQLLPDMGGVSEQVEDEIAAAAMAVGASHAGAKAMSGSSGGGFALMSEPLGLAEMTETPLVLLEAQRAGPSTGMPTKPEQADLEHVLYTSQGDSHRVAFGPKDPKECYEQTRTAFEIAYDYQIPVILLYDQKLSGEYRNVDASFFDREPAADLGTTLSEDQIPDAPHDPTGKYHRYQHDVEDGVSPRTIPGQSGGRYLASGNEHWPNGHISEDTDNRVAQVERRLQKLAAIRDDLDERDQQTHYGDEDADIGLIAWGSQEGTVEEAVHRLNDDGNSVKALGISDLAPFPVAETRAFVDSVDEAIVVEMSSTKQFRGLIQKEVGDIGGKLSSLLKYNGNPFEPAEIVEAVEIEQAGDGAEPAAQTTLEPAAGD

SEQ ID NO: 14 - protein, 312 aa, Halobacterium sp. NRC-1
MSKAFSAIDEDREVDRDAFTPGVEPQPTWCPGCGDFGVLKALKGAMAELGKDPEEILLATGIGCSGKLNSYFDSYGFHTIHGRSLPVARAAKLANHDLEVVAAGGDGDGYGIGGNHFMHTARENHDITYIVFNNEVFGLTKGQTSPTSPKGHKSKTQPHGSAKSPIRPLSLSMTSGASYVARTAAVNPNQAKDILVEAIQHDGFAHVDFLTQCPTWNKDAKQYVPYVDVQESDEYDFDVTDRREAQELMTETEEALYDGTVLTGRYYQDEQRPSYQAEKQSRGDMPEEPVAKRYFDDDYEWERSFDVIDRHK

SEQ ID NO: 15 - DNA, 2864 bp, Halobacterium sp. NRC-1
atgccatattggtccacggctgggccagaccagattatgactgacgacgaactcatctggcgaatcgcagggggttccggagacgggatcgactcgacaagccagaatttcgccaaagcgctgatgcgctcgggcctcgacgtcttcacgcaccgccactacccgtcgcggatccgcggcggccacacgtacgtggagatccgggcgcgggacggtaccgtaacctcccgcggtgacggctacaacttcctgctcgcgctcggcgactcgttcgcccgcaacccgagcgaggaggccgtctacggcgacgaggaagtgaagccgctcactgagaacctcgacgacctgcgcgcgggcggcgtcatcatctacgacgaggggctgctcgacgacgaggacgtcggcgacctcgaacagcaggccgacgccaacgactggcatctctacccgcttgacctgcgcgggctcgccaaggaacacggccgcgaggtcatgcgcaacaccgcgggcgtcggcgccaccgcggcgctcatcgacatggacctcgaccacatcgaggacctgatgagcgacgccatgggcggcgacatcctcgaacagaacctcacggtgctccgcgacgcctacgagcaggtgtcggaaatggagcacacccacgacctatcggtgccgaccgggagccacgacgagccacaagtgctcatgtccgggagccacgcgatcgcgtacggcgcgatcgacgccggctgccggttcatctccgggtatccgatgacgccgtggacggacgcgttcacgatcatgacccagctgttgcccgacatgggcggggtctccgagcaggtcgaagacgagatcgcggcggcggcgatggcggtgggtgcaagccacgccggcgcgaaggcgatgtccggctcctccggcggcgggttcgcgttgatgagcgagcccctgggcctcgcggagatgaccgagacgcccctggtgttgctggaagcccagcgcgccgggccgtccacgggcatgccgacgaagcccgagcaggccgacctggagcacgtgctgtacaccagccagggggacagccaccgcgttgcgttcggccccaaagaccccaaggagtgttacgagcagacccgcacggcgttcgagatcgcgtacgactaccagatccccgtgatcctgctgtacgatcagaagctctccggggagtaccggaacgtcgacgcgtcgttcttcgaccgcgagccggcggcggacctcgggacgacgctctccgaggaccagatccccgacgcgccacacgacccgacggggaagtaccaccgctaccagcacgacgtcgaggacggcgtcagcccccggacgatcccggggcagtccggcggtcggtatctcgcctccggcaacgagcactggccgaacggccacatcagcgaggacaccgacaaccgcgtggcgcaggtcgagcgccgcctccagaagctggcggcgatccgcgacgacctcgacgagcgcgaccagcagacccactacggcgacgaggacgccgacatcggcctcatcgcgtggggcagccaggagggcaccgtcgaggaagcggtccaccggctgaacgacgacggcaacagcgtgaaggcgttggggatcagcgacctcgcgccgttccccgtcgcggagacgcgggcgttcgtcgacagcgtcgacgaagccatcgtcgtggagatgtcctccaccaagcagttccgtggcctcatccagaaggaggtcggagacatcggcgggaagctgtcgagtctcctgaaatacaacggcaacccgttcgagcccgcggagatcgtcgaggccgttgagatcgaacaggccggcgacggcgcggagccggccgcccagaccacactcgaacccgcagcaggtgactgataatgagtaaggcattcagcgcgattgatgaggaccgcgaggtcgaccgggacgcgttcacgcccggcgtcgaaccgcagccgacgtggtgtcctggctgtggtgacttcggtgtcctgaaggccctgaaaggggcgatggcggagctcggcaaggaccccgaggagatactgcttgcgaccgggatcggctgttccgggaagctcaacagctacttcgacagctacggcttccacacgatccacgggcgctccctgcccgtggcccgcgccgcgaagctggccaaccacgacctggaggtcgtggccgccggcggtgacggcgacggctacgggatcggcggcaaccacttcatgcacaccgcccgggagaaccacgacatcacgtacatcgtgttcaacaacgaagtgttcggcctgacgaagggccagacatcgccgacgagccccaaggggcacaagtccaagacccagccccacggctccgcgaagtccccgatccgaccgctctcgctgagcatgacctcgggggcgtcgtacgtggcgcgaaccgcggccgtgaaccccaaccaggcaaaggacatcctcgtggaagccatccagcacgacggcttcgcgcacgtggacttcctgacgcagtgtccgacctggaacaaggacgccaagcagtacgtcccgtacgtggacgtccaggagtccgacgagtacgacttcgacgtcacggaccggcgggaggcacaggagctgatgaccgagaccgaggaagccctctacgacgggaccgtgctgaccggccggtactaccaggacgagcagcggccgtcgtatcaggccgaaaagcagtcccgcggggacatgcccgaggaaccggttgcaaagcggtacttcgacgacgactacgagtgggagcgctcgttcgacgtcatcgaccgccacaagtaa

SEQ ID NO: 16 - protein, 607 aa, Hydrogenobacter thermophilus TK-6
MAFDLTIKIGGEGGEGVISAGDFLTESAARAGYYVVNFKSFPAEIKGGYAQSTIRVSNKKLYTTGDGFDILCCFNGEAYEFNRKHLRPGTVLVYDSSDFEPEEHEGVVMYPVPLSHLAKDIMKAYITKNVIALGVLCGLFDIPVQSIKDSIKAKFLRKGQEIIELNYKALETGINYVRENIKKLDGYLFPPAKEPKDVVIMEGNQAIAKGAVVAGCKFYAAYPITPATTVGNYIVEDLIRVGGWLYQAEDEIASLGMALGASFAGVKAMTATSGPGLCLMTEFISYAGMTELPIVIVDVQRVGPATGMPTKHEQGDLYHAIYSGHGEIPRAVLAPTNVEESFYLTVEAFNLAEKYQIPVIVLTDASLSLRAEAFPTPKVKDIKVINRWVYNAEDDPEGKFRRAGRFLRYALFTEDGITPMGVPGDPNAIHAITGLERQENSDPRNRPDIRTWQMDKRFKKMEKLLREDAEKFYEMDAPFEKADIGIISWGLTASATKEAVERLRSKGRKINALYPKLLWPLRVDILENFAKSCRRIIMPESNYSGQLATVLRAETRIRPISYCIYRGEPFIPREIEEFIEYVLENSYIEEGKFTPANLYGEKAYGLI

SEQ ID NO: 17 - protein, 295 aa, Hydrogenobacter thermophilus TK-6
MLEVHLKPADYKSDVEPTWCSGCGDFGVVAALTRAYSELGLKPENIVSVSGIGCSSRLPLFVKNYSVHSLHGRAIPVAVGIKLARPDLTVIVETGDGDLFSIGAGHNPHAARRNIDITVICMDNQVYGLTKNQVSPTSREGLYGSLTPYGSIDRPVNPIATMLSYGATFVAQTYAGNLKHMTEVIKQAIQHKGFSFVNVISPCPTFNKVDTFQYYKGKVKDINEQGHDPSDYRKALELAFHDLDHYHDPNAPVPIGVFYKAELETYEDRMQSVKRRYKQVEDVQELIDMCKPKAL

SEQ ID NO: 18 - DNA, 2725 bp, Hydrogenobacter thermophilus TK-6
atggcgtttgatttgaccatcaaaataggtggtgaaggtggtgaaggtgttatatccgccggggattttttgacggaatctgcagcacgggctggttattatgtggttaactttaagagcttccccgcggagataaagggtgggtatgcccagtccaccatcagagtctccaacaaaaagctttacacaacaggagatggctttgacattctgtgctgttttaatggtgaggcttacgaatttaacaggaagcatttaaggccgggtacggtgctcgtttatgactcttcggattttgagccggaggagcacgagggtgtggtcatgtatccggttcccctctcccatctggcaaaggacataatgaaggcttacataacaaagaatgtaatagctctgggtgttctctgtgggctgtttgatatacctgtgcagtctataaaagactcaataaaagcaaagtttttaagaaagggacaggagataatagaactaaactataaggctctggagacgggtataaactatgtcagggagaatataaagaaattggatggataccttttccctcctgcaaaggaaccaaaagatgtggtaatcatggagggcaatcaggcaatagccaagggtgcggtggtggcaggctgtaagttttatgcagcttatcccataacgccggcaacgacggtaggaaactacatagtagaagacctcataagggtgggaggttggctctatcaagctgaggatgaaatagcctccctcggtatggctttaggggcttcttttgcaggcgtaaaagctatgaccgccacctccggaccgggattatgccttatgacggagtttatctcttacgcaggtatgacggagcttcccatagtgatagtggatgtgcagagggtaggacctgcaacgggtatgcctaccaagcacgaacagggagacctctaccacgccatatactcagggcacggtgagataccaagggcagtgcttgctcccaccaatgtggaagagagcttttaccttactgtggaggctttcaatctggcggaaaagtatcagatacccgttatagttctgacggatgcatccctttctctgagagcggaagccttccctactccaaaggtaaaggacattaaggtgataaacagatgggtctataatgcagaagatgaccccgagggtaagttcagaagagctggaagatttcttaggtatgccctttttaccgaggacggcataacgcctatgggtgtacccggagaccccaacgccatacacgccataacggggcttgagcgtcaagaaaactcagacccaagaaacagacctgacataagaacatggcagatggacaaaaggtttaagaagatggaaaagctcctgagggaagatgcggaaaagttttacgagatggatgcaccctttgagaaggctgacataggtatcatatcctggggtcttaccgcatccgctacaaaggaggctgttgagagactaaggagcaaaggtagaaaaataaacgccttgtatcccaagctcctctggccactcagggtggatatactggaaaactttgcaaaaagctgtaggagaataatcatgcctgagagtaactacagcggtcagcttgcaactgtgcttagggctgaaacgcgtataagacctataagctactgcatatacaggggagaaccctttataccgagggagatagaggagtttatagagtatgtactggagaactcttacattgaggagggcaaatttacacctgcaaacctttacggcgaaaaggcttacggactaatttaaaggaggtgtaagtatgttagaagttcacttaaaacctgcagactacaagagcgatgtagaacccacctggtgttcgggatgcggtgattttggtgtggtggcggctctaactagagcttattcggagcttggattaaagcctgaaaacatagtttccgtatccggtataggttgttcctcaaggcttcccctctttgttaaaaactactcggtgcattcactgcacggaagagctatcccagtagctgtaggcataaagctggcaaggccggaccttaccgtcatagtggaaacgggcgacggagacctcttctccataggcgcgggacacaacccacacgcagcacgcagaaacatagacataaccgtcatatgtatggacaatcaggtttatggtcttaccaaaaatcaagtttctccaacttcaagggaaggactttacggctccctaacaccttacggctccatagacagacctgtaaaccccatagccaccatgctctcctacggtgccacctttgttgcacagacttatgcgggcaatctcaagcacatgacagaggtgataaagcaagctatacagcataaaggcttttcctttgtaaatgtgatatctccctgccccacctttaacaaagtggacaccttccagtactataagggtaaggtgaaggacataaacgagcagggacacgacccatccgattacagaaaggctcttgaacttgctttccatgaccttgaccactatcacgatccgaacgctccagtacctataggcgtattttacaaagctgagctggaaacctacgaagacaggatgcagtccgtgaagagaaggtacaaacaggtggaagatgtgcaagaactcatagatatgtgtaagccaaaagctttatga

SEQ ID NO: 19 - protein, 578 aa, Bacillus sp. M3-13
MINQLSWKVGGQQGEGIESTGEIFSIALNRLGYYLYGYRHFSSRIKGGHTNNKIRVSTTQVRSISDDLDILVAFDQETIDVNYHELREGGVVIADAKFKPSIPEDGKATLYAVPFTEIATELGTSLMKNMVAVGASSAILDLDAESFREVVQEIFGRKGESIVEKNMEAIRAGVQFIKDQAENLETMQLAKADGNKRLFMIGNDAIALGAVAAGSRFMPAYPITPASEIMEYLIKKLPKFGGTVIQTEDEIAACTMAIGANYAGVRTLTASAGPGLSLMMEAIGLSGMTETPLVVVDTQRGGPSTGLPTKIEQSDLMAMIYGTHGEIPKVVMAPSTVQEAFYDTIEAFNIAEEYQVPVILLTDLQLSLGKQSVEALDYKNIEIRRGKLDINQELPAADDKAYFKRYEVTEDGVSPRVIPGMKHGIHHVTGVEHEETGKPSEVAANRQAQMDKRLRKLNNLKFNTPVHVNAKHEEADVLLVGFNSTRGTIEEAMERLELEGVKANHAQVRLIHPFPTEEIAPLVKAAKKVIVVEYNATGQLANILKMNVGEHEKIRSLLKYDGDPFLPKEIHTKCKELL

SEQ ID NO: 20 - protein, 288 aa, Bacillus sp. M3-13
MATFKDFRNNVKPNWCPGCGDFSVQAAIQRAAANVGLEPENLAVVSGIGCSGRISGYINSYGFHGIHGRSLPIAQGVKMANKDLTVIASGGDGDGFAIGLGHTIHAIRRNIDVTYIVMDNQIYGLTKGQTSPRSEVGFKTKSTPQGSIESSLSVMEMALTAGATFVAQSFSTDLKDLTSLIEQGIKHKGFSLINVFSPCVTYNKVNTYDWFKENLTKLADIEGYDAHNKVSAMQTLMEHNGLVTGLIYQNKDQQSYQDLVPNYSEEPLAKADLQLDEEQFNALVKEFM

SEQ ID NO: 21 - DNA, 2604 bp, Bacillus sp. M3-13
atgatcaatcaactttcatggaaagttggagggcaacaaggggaaggtatcgaaagtaccggtgagattttctccattgcattaaatcgtttaggctattatttatatggttatcgccatttttcttctcgtattaaaggtggacatacgaacaacaaaattcgtgtgagtacgactcaggtccgttccatttcggacgaccttgatatattagtagcgtttgatcaagaaacaatcgacgtaaactatcatgaactccgcgaaggtggagtggtaattgcagatgcaaagtttaaaccaagcatacctgaagacgggaaagctacattgtacgctgtaccattcactgaaattgctactgagcttggaacatcattgatgaagaacatggttgctgtcggagcttcaagtgccatccttgatttagatgcggaatcattccgtgaagtggtgcaagaaattttcggacgcaaaggcgaatccattgttgagaaaaacatggaagcgatccgagcaggtgttcaattcattaaagatcaagctgaaaatttagaaacaatgcagcttgcaaaagcagacggcaataaacgactattcatgatcggtaatgatgcgattgcattgggtgcagttgctgcaggatctcgttttatgccggcttacccaattactccagcatctgaaattatggaatacttaatcaaaaagcttccaaaattcggcggtactgtgattcaaacggaagatgagattgctgcttgtaccatggcaattggtgccaactatgcaggtgtacgtactttgactgcttcagcaggcccgggactatccttaatgatggaagcaattggactttctggtatgacagaaacaccgcttgtagttgtggacacgcaacgtggaggaccaagtacagggttaccgacaaagattgagcagtctgaccttatggcgatgatctatggtactcacggagagatcccgaaagtggtaatggctcctagtactgtacaagaggctttctacgatacaatcgaggcatttaacattgcagaagaatatcaagtacctgtcattcttttaactgatcttcaattgtctctagggaagcaatcggtagaagcattagattacaaaaacattgaaattagacgcggaaagctggatatcaatcaagagcttccggctgctgacgataaagcatatttcaaacgatatgaagtaacagaagatggcgtatctccccgtgtgattcctggcatgaaacacggtatccatcacgttactggtgtagagcacgaagagacaggtaagccttctgaagttgctgcgaaccgtcaagcacagatggacaagcgtcttcgtaaattgaataaccttaaattcaatacgcctgttcatgttaatgcaaagcatgaagaagcggatgtactacttgttggatttaactcgacgcgcggaacgatcgaagaggcaatggaaagattggaattggaaggtgttaaagctaaccatgcacaagtccgcctgatccacccattcccgacagaagaaatcgcgccactggtaaaagcggctaaaaaagttattgttgtggagtataacgctactggacaacttgcaaacatccttaaaatgaatgttggcgagcatgagaaaatccgtagtctcttaaagtatgatggggatccattcttaccgaaagaaatccacacaaaatgcaaggagttgttataaatggcaacgtttaaagactttcgaaataatgtaaaacctaactggtgccctgggtgtggagacttctcggtacaagctgccattcaacgtgctgccgcaaatgttggtttagagcctgaaaatcttgcagtagtatctggaatagggtgttctggacgtatttccgggtacatcaattcctacggtttccatggtattcatggtcgctctctaccaatcgcacaaggtgtgaaaatggcgaataaagatcttacggttatcgcttcaggtggagatggagatggatttgccatcggtttaggtcataccatccatgcaattcgtcgaaatattgatgttacatacatcgttatggataatcagatttatggactaacaaaaggccaaacatcaccacgtagtgaagtaggattcaaaacaaaatctacaccacaaggttccattgaatcctcactgtctgtaatggaaatggctttaacagcaggagcgacatttgtagcgcaaagcttctctactgatttgaaagacctaacttccttgatcgaacaaggaatcaagcataaagggttctctctaattaacgtgtttagcccgtgtgttacatataataaagtgaacacatatgactggtttaaagaaaatttgacaaaattggctgacattgaaggttatgacgctcacaacaaagtttctgcgatgcagacactaatggagcataatggcctagtaactggtttgatctatcagaataaggaccaacagtcttatcaagatttggttcctaattatagcgaagagcctcttgcaaaagcagatcttcaattagacgaagaacaattcaacgcactagtaaaagaattcatgtaa

SEQ ID NO: 22 - protein, 582 aa, Paenibacillus larvae subsp. larvae B-3650
MISQLSWKIGGQQGEGVESTDRIFSTALNRLGYYLYGYRHFSSRIKGGHTNNKIRISTKPIRSISDDLDILVAFDQESIDLNAHELRENAVVVADAKFNPTLPEGINARLFPVPITAIAEELGTSLFKNMAASGASWALLGLPLEVFNKAVEEEYGRKCAAVVEKNIEAVKRGAEYVLDLAGGPLEEFRLEPADGKQKLFIIGNDAIGLGAVAAGCRFMPAYPITPASEIMEYLIKVLPKYGGTVIQTEDEIAACTMAIGANYGGVRAMTTSAGPGLSLMMEAIGLAGMTEIPVVIVDTQRGGPSTGLPTKQEQSDINAMIYGTHGEIPKIVIAPSTIEECFYDTVEAFNLAEEYQCPVIVLTDLQLSLGKQSSELLDYNKISINRGKLVHELEPAEPNTMFKRYEFTEDGISLRVLPGTKYGIHHVTGVEHDQTGRPNEGTDNRKKMMDKRLRKLTNVKVTNPIHVDAPHEEPDVLIIGIGSTGGTIDEARGRLDKDGLKTNHITVRLLNPFPAEELRPYMEKAKTVVVVENNATAQLANLIKLHVGFADKIKNLLKYNGNPFLPSEIYQEVKELNVTWQH

SEQ ID NO: 23 - protein, 288 aa, Paenibacillus larvae subsp. larvae B-3650
MATLKDFRNNVKPNWCPGCGDFSVQASIQRAAANVGLEPEQLAIISGIGCSGRISGYVNAYGLHGVHGRALPIAQGVKMANRELTVVAAGGDGDGFAIGMGHTVHAIRRNIDITYIVMDNQIYGLTKGQTSPRSGEGFKTKSTPQGSIETPLAPLEMALAAGATFVAQSFSSNLKQLTHVIEEGIKHKGFSIINVFSPCVTFNKVNTYDWFKEHVVNLDDLPDYDPSNRIQVMTKLMETEGMLTGIIYQDTSKPSYEQLVPGFKEEALAKQDIHLSEEEFDKLVAEFK

SEQ ID NO: 24 - DNA, 2603 bp, Paenibacillus larvae subsp. larvae B-3650
atgattagtcagctatcgtggaagatcgggggacaacaaggtgaaggggtggaaagcaccgatcgtattttttccacagcattgaaccgccttgggtattatttgtatgggtatcgtcatttctcttctcggattaaagggggacatacgaacaacaaaattcggatcagtacaaagccgattcgatcgatctcggatgatctggatatccttgtagcgtttgaccaagaatccattgatttaaatgcacatgagcttcgggagaatgcagttgttgtggctgatgccaaatttaacccgacattgcctgaagggatcaatgcgcgcttgtttccagtaccgattacagcgattgcagaagaacttggaacgtctcttttcaaaaacatggccgcttcaggcgcatcatgggctttgcttggtcttccattggaagtattcaacaaagcggtagaagaagagtatggccgtaagtgtgcagcagtagttgagaaaaacattgaagcagttaaacgcggagctgagtatgtgcttgatcttgctggaggtcctcttgaagaatttagacttgagccggctgacggtaaacaaaaactgtttattatcggaaatgatgctatcgggcttggcgcagttgcggcgggttgccgtttcatgcctgcatatccgatcaccccagcttccgaaataatggaatatttgattaaagtgcttcctaaatatggcggaactgttatccaaacggaggatgaaattgccgcctgtacgatggcgatcggggcgaactacgggggagtacgtgcaatgaccacttctgcgggaccgggtttgtcactgatgatggaagcgattggtcttgccggaatgacagaaataccggtcgtgattgtggatacccaacgcggaggcccaagtacaggattgccgacaaagcaggaacaaagtgatattaatgcgatgatttacggaactcatggagaaattcctaaaattgtcatcgcacctagtacgattgaagaatgtttctatgatacggtagaggcatttaacttggccgaagaatatcaatgcccggttatcgttttaacagatttgcaactttctcttggcaaacaatcatccgaactgctggattataacaagatctccattaaccgggggaaattggtacatgaattagagcctgccgagcctaatacaatgttcaaacgttatgaatttacggaagatggaatatctctgcgtgttcttcccggaacgaagtatggtattcatcatgtaacaggtgttgagcatgatcaaaccggacgtccgaatgagggaacggataaccggaaaaaaatgatggataaacgccttagaaaattaacaaatgtcaaggtgactaatccgattcatgtggatgcgccgcatgaagaaccggatgtgctaattattggaatcgggtccacaggcggtacgatagatgaagccagaggacgtcttgacaaagacgggctaaaaactaatcacattactgttcgcctgctgaacccattcccggcggaagagctccgcccttatatggaaaaagccaaaactgtagtagttgtagaaaacaacgcaactgcacagctggctaatctgatcaagcttcatgtaggatttgcggataaaattaaaaacctgctgaaatataacgggaatccgttcttaccgtctgaaatctaccaagaagtcaaggagctgaatgtaacatggcaacattgaaagattttcgtaacaacgtaaagccgaactggtgtccaggatgcggggacttttccgtacaggcgtccatccagcgtgctgcggccaatgttggattggaaccggaacagcttgctattatttccggaatcggttgttcaggccggatatccggttatgtaaatgcatacggtctccacggtgttcatggtagagctcttccaatcgctcagggagttaaaatggcaaaccgagaattgactgttgtagccgcaggcggtgacggggacggatttgccatcggcatgggtcatacagtacatgccatccgccgtaatattgatataacttacattgtcatggataatcaaatctatggattgacgaaaggccagacctctccgcgaagcggtgagggcttcaaaacaaaaagtacaccccaagggtccattgagactccattggcaccacttgagatggctcttgcggcaggagcgactttcgtagcccagtctttctccagcaatctgaagcagctgacgcacgtgattgaagaaggtatcaaacataaaggattttctattattaatgtattcagtccttgtgtaaccttcaacaaggtaaatacgtacgactggttcaaagaacatgtggtgaatttagatgatttacctgattatgatccttcaaaccgtattcaggtcatgacaaagctcatggaaacagaagggatgctaaccggaattatttatcaggatacaagtaaaccttcctatgagcagctcgttcctggatttaaggaagaagctctcgcaaaacaagatattcatctgagtgaggaagagtttgacaaattggtagcagagtttaaataa

SEQ ID NO: 25 - protein, 584 aa, Haladaptatus paucihalophilus DX253
MQDLNWAIGGEAGDGIDSTGKIFAQALSRAGRHVFTSKDFASRIRGGYTAYKIRSSTDRVESVVDRLDILVALTQRTIDENLDELHEDSVIIYDGERTEMEDVDIPEEMIGLAVPLRSLAKDAGGTIMQNTVALGAACEVANFPIENLDSALDKKFGAKGEAIVENNKEAARLGQEYVQEEYDYDFEYDVETTDNDYVLLNGDEAIGMGAIAAGCRFYSGYPITPATNVMEYLTGRIEHFGGTVMQAEDELSAINMALGAARAGARSMTATSGPGIDLMTETFGLIAQSETPLVICDVMRSGPSTGMPTKQEQGDLNMTLYGGHGEIPRFVVAPTNVAECFHKTVEAFNFAEKYQTPVFLLADLAMAVTEQTFSPEEFDMDSVEIERGNIVDEDDIEAWTDEKDRFQPHFPTADGISPRAFPGTKGGAHMSTGLEHNALGRRTEDTEIRVEQVDKRNRKVETAQEEEDWSPREFGDEDADTLVISWGSNEGPMREALDFLEEDDVSVRFLSVPYIFPRPDLTEDIESADTVIVVECNETGQFANVLEHDALTRVERINKYNGIRFKADELADDIKAKLGQEVEA

SEQ ID NO: 26 - protein, 287 aa, Haladaptatus paucihalophilus DX253
MSSEVRFTDFKSDKQPTWCPGCGDFGTMNGMMKALAETGNSPDDTFVVAGIGCSGKIGTFMHSYAIHGVHGRALPVGTGVKLANPDLEVMVAGGDGDGYSIGVGHFIHAVRRNVDMSYVVMDNRIYGLTKGQASPTSREDFETSTTPEGPQQPPVNPLALALSAGATFIAQSFSTDAQRHAEIVQKAIEHDGFGFVNVFSPCVTFNDVDTYDYFRDSIVDLADEGHDPHDYEAAKEKILDASKEYQGVIYQDEDSVPYSELHGIEGNMSEIPDGAPEDAMDLVREFY

SEQ ID NO: 27 - DNA, 2615 bp, Haladaptatus paucihalophilus DX253
atgcaagacctgaactgggccatcggcggcgaagccggcgatggaatcgattcgaccgggaaaatctttgcgcaggcactctcccgagcgggccgacatgtcttcacgtcgaaggatttcgcgtcccgtattcgagggggctacaccgcgtacaagatccggtcgtctaccgaccgagtcgagagcgtcgtcgaccgactggacatcctcgtggcactgacccagcggaccatcgacgagaacctcgacgaacttcacgaggacagcgtgatcatctacgacggggaacggacggagatggaggacgtcgacatccccgaggagatgatcggattggccgttccgctccgcagtctggcgaaggacgcgggtggaaccatcatgcagaacaccgtcgcgctcggtgcggcgtgtgaagtggcgaacttccccatcgagaacctcgacagcgcgctcgacaagaagttcggcgcgaagggtgaggccatcgtcgagaacaacaaggaagccgcccgtctcggacaggagtacgtccaggaggagtacgactacgacttcgagtacgacgtggaaacgacggacaacgactacgtcctgctcaacggtgacgaggccatcggcatgggtgctatcgccgctggctgtcgcttctactccggctaccccatcacgcccgcgacgaacgtcatggagtatctcacgggccgaatcgagcacttcggcggcacggtgatgcaggccgaggacgaactgtcggccatcaacatggcgctcggcgcggcgcgcgctggcgcacgctcgatgacggcgacgtccggtccgggtatcgacctgatgaccgagacgttcggtctcatcgcacagagcgagacgccgctcgtcatctgcgacgtgatgcgctccggtccctcgaccgggatgccgacgaaacaggaacagggcgacctgaacatgacgctgtacggcggccacggcgagattccgcggttcgtcgtcgcgccgacgaacgtcgccgagtgtttccacaagaccgtcgaggcgttcaacttcgccgagaagtaccagacccccgtcttcctgctcgccgacctcgccatggccgtcaccgagcagacgttctcgcccgaggagttcgacatggattccgtcgaaatcgagcgcggaaacatcgtggacgaggacgacatcgaggcgtggacggacgagaaggaccggttccagccccacttcccgaccgctgacggcatcagcccgcgcgcgttccccggaacgaagggcggtgcccacatgtccaccggtctcgaacacaatgcgctcggtcggcggaccgaggacaccgaaatccgcgtcgagcaggtcgacaagcgaaaccgcaaggtcgagacggcacaggaagaagaagactggagtccgcgcgagttcggcgacgaagacgccgacacgctcgtcatctcgtgggggtcgaacgaagggccgatgcgcgaagccctcgacttcctcgaagaggacgacgtgagcgttcggttcctctcggttccgtacatcttcccccgccccgacctcaccgaggacatcgagtccgcggacaccgtcatcgtggtcgagtgtaacgaaaccgggcagttcgccaacgttctcgaacacgacgcgctcactcgtgtcgagcggataaacaagtacaacggtattcgattcaaggccgacgagttggccgacgacatcaaagcgaaactcggacaggaggtagaagcatgagttcagaggttcgattcaccgacttcaagtcggacaagcaaccgacgtggtgtcccggatgcggcgacttcgggacgatgaacgggatgatgaaggcactcgccgaaaccggcaacagcccggacgacacgttcgtcgtcgcgggtatcggctgttccggaaaaatcgggacgttcatgcactcctacgcgattcacggcgtgcacgggcgtgcgcttcccgtcggcaccggcgtcaaactcgccaaccccgacctcgaagtgatggtcgcgggcggcgacggtgacggctactccatcggtgtgggtcactttatccacgccgtgcgccggaacgtggacatgtcctacgtcgtcatggacaaccgcatctacgggctgacgaagggacaggcctcgccgaccagccgcgaggacttcgagacgagtacgacgccggaaggcccgcaacagcccccggtcaacccgctcgccctcgccctctcggcgggtgcgacgttcatcgcacagtccttctcgaccgacgcacagcgacacgccgaaatcgtccagaaggccatcgagcacgacggcttcggcttcgtgaacgtcttctcgccctgcgtcacgttcaacgacgtggacacgtacgactacttccgcgactccatcgtcgacctcgcggacgagggtcacgacccgcacgactacgaggcggccaaagagaagattctcgacgccagcaaggagtatcagggcgtcatctaccaggacgaagatagcgttccgtacagcgaactccacggcatcgagggcaacatgtccgagattcccgacggcgcacccgaggacgcgatggacctcgtgcgcgagttctactga

SEQ ID NO: 28 - protein, 573 aa, Magnetococcus sp. MC-1
MEKKDLIIRVAGEGGEGIISSGDFIAAACARAGLEVYTFKTFPAEIKGGYAMYQVRASSEKLYCQGDTFDVFCAFNGEAYEQNKDKIKPGTAFVYDYPGGDFEPDEIPEGVFAYPIPMSQTAKEMKSYRSKNMVALGALSELFNISENTLKEVLSDKFGKKGEEVLAFNLEAFDKGKALAKALTKADPFRVADPQEPKDVIIMAGNDAVGLGGILGGLEFFSAYPITPATEVAKYVATHLPKCGGDLVQAEDEIASIAQVLGASYAGKKSMTATSGPGLALMSEMLGMAHMSETPCLVVDVQRGGPSTGLPTKHEQSDLFLAIHGGHGDSPRIVLSVEDVKDCISMTVDGLNLAEKYQAPVIVLSDGSLAFSTQTIPRPKPEDFTIINRKTWDGQGTYKRYELTEDNISPMAAPGTPNAKHIATGLEHGETGAPNYSPANHELMHRKRFNKQNSVLDFYKNMEVEGVEGEADVGIITWGSTIGVVREAMQRLTAEGLKVKAMYPKLLWPMPVADYDAFGATCKKVIVPEVNFQGQLSHFIRAETSIKPIPYTICGGLPFTPEMIVNRVKEEIQ

SEQ ID NO: 29 - protein, 292 aa, Magnetococcus sp. MC-1
MTVEAFHKMENMKPKDYKSEVPTTWCPGCGHFGILNGVYRAMAELGIDSTKFAAISGIGCSSRMPYFVDSYKMHTLHGRAGAVATGTQVARPDLCVVVAGGDGDGFSIGGGHMPHMARKNVNMTYVLMDNGIYGLTKGQYSPTSRPEMTAYTTPYGGPENPMNPLLYMLTYGATYVAQAFAGKPKDCAELIKGAMEHEGFAYVNIFSQCPTFNKIDTVDFYRDLVEPIPEDHDTSDLGAAMELARRPGGKAPTGLLYKTSAPTLDQNLAKIRERLGGHVGYDKNKIIALAKP

SEQ ID NO: 30 - DNA, 2597 bp, Magnetococcus sp. MC-1
atggagaagaaagatctgattatccgcgtggcaggtgaggggggggaaggtatcatctcctccggtgacttcattgctgccgcatgtgcgcgggctggtttggaggtctacacctttaaaaccttcccggcggaaatcaagggcgggtacgcaatgtatcaagtccgtgccagtagcgagaagctctattgtcagggtgacacctttgacgtgttctgcgcctttaatggcgaagcttatgagcagaacaaagataagattaaacccggcaccgcttttgtctatgactatccaggcggtgattttgaacctgacgagatccctgagggtgtgtttgcatacccgatccccatgtcacaaacagcgaaggaaatgaaatcctaccgctccaaaaacatggtggctctgggtgctctgtcggagttgtttaacatctcagagaacacgcttaaagaggtgttgagcgacaagtttggtaaaaaaggcgaagaggttttggcgttcaacctagaagcttttgataagggtaaagcgctggcaaaggctctcaccaaagcggatcctttccgtgtggcggatccgcaagagcctaaagatgtgatcatcatggcgggtaacgatgccgtgggtctgggtggcattttgggtggcttggagtttttctctgcctatcccattacccccgcgaccgaggtggccaagtatgtggcgactcacctgcctaagtgtggtggggatttggtgcaggctgaggatgagatcgcctctatcgcgcaggtgttgggtgcctcttatgcgggtaaaaaatccatgactgccacctctggtcctggtctggcgctcatgtccgagatgttgggcatggcccacatgtctgagaccccctgtctggtggtggatgtgcaacgtggtggtccatccacgggtctgcccactaagcatgagcagtcggatctgtttttggccattcatggtggtcatggcgactccccgcgtattgtgctctcggtggaagatgtgaaagattgcatcagcatgactgtggacggtctgaatttggctgagaaatatcaggcccccgtgattgtgctctccgacggctctctggccttctctacgcagaccattccccgccctaaacccgaagattttaccatcatcaatcgtaaaacctgggatggccaaggcacctataagcgttatgagttaaccgaagataacatctccccgatggcggctcccggtacccctaatgccaagcacattgccacgggtctggagcatggtgaaacgggtgcgcccaactattcgcctgccaaccatgagttgatgcatcgcaagcgcttcaacaagcaaaactctgtgttagatttttataaaaacatggaagttgagggggttgagggcgaagcggatgtgggcattatcacttggggttccaccatcggggtggtgcgtgaggcgatgcaacgtttgaccgcagaggggctgaaggtcaaggcgatgtatcccaaattgctgtggccaatgccggttgcggactatgatgcctttggtgccacctgtaaaaaggtgattgtccctgaggtcaacttccaggggcagctttcccactttatccgtgcggaaacgtccattaagcccattccttacacgatctgtggcggtttgccgttcacacctgagatgattgtgaaccgggttaaggaggagatccaatgactgtcgaagccttccacaagatggaaaatatgaagcccaaggactacaagtccgaggttcccaccacatggtgcccaggttgtggccactttggtattctgaacggtgtctaccgtgcgatggcagagttgggcattgactcaaccaaatttgccgccatttccggtattggctgctcgtcacgtatgccatacttcgttgactcctacaaaatgcacaccctgcacggtcgtgctggtgcggtggcaacgggtacccaggttgcgcgtcctgatctgtgcgtggtggtggcgggtggtgatggcgatggtttctccatcggtggtggtcacatgccccacatggcgcgtaaaaatgtcaacatgacctacgtgctcatggataatgggatctatggtttgaccaagggtcaatactctccgacctcgcgtccagagatgacggcctataccaccccttatggtggtcctgagaatcccatgaacccgctgctctacatgctcacctatggtgcgacctatgtggcccaggcttttgccggcaagcccaaggattgtgcggagttgatcaagggtgccatggagcatgaagggtttgcttatgtgaacatcttctctcagtgccccacctttaacaaaattgacacggtggatttctatcgtgatctggtagagcctatccctgaggatcatgatacttccgatcttggggccgcgatggagttggctcgtcgtccgggtggtaaagccccgactggcctgttgtacaaaacttcagcaccaaccttggaccagaacttggccaaaattcgtgagcgccttggtggtcacgtgggctatgataagaacaagatcattgccctggcaaagccgtaa

SEQ ID NO: 31 - protein, 472 aa, Hydrogenobacter thermophilus TK-6
MFKKVLVANRGEIACRVIRACKELGIQTVAIYNEIESTARHVKMADEAYMIGVNPLDTYLNAERIVDLALEVGAEAIHPGYGFLAENEHFARLCEEKGITFIGPHWKVIELMGDKARSKEVMKRAGVPTVPGSDGILKDVEEAKRIAKEIGYPVLLKASAGGGGRGIRICRNEEELVRNYENAYNEAVKAFGRGDLLLEKYIENPKHIEFQVLGDKYGNVIHLGERDCSIQRRNQKLVEIAPSLLLTPEQREYYGSLVVKAAKEIGYYSAGTMEFIADEKGNLYFIEMNTRIQVEHPVTEMITGVDIVKWQIRIAAGERLRYSQEDIRFNGYSIECRINAEDPKKGFAPSIGTIERYYVPGGFGIRVEHASSKGYEITPYYDSLIAKLIVWAPLWEVAVDRMRSALETYEISGVKTTIPLLINIMKDKDFRDGKFTTRYLEEHPHVFDYAEHRDKEDFVAFISAVIASYHGL

SEQ ID NO: 32 - protein, 652 aa, Hydrogenobacter thermophilus TK-6
MQAVEIMEEIREKFKEFEKGGFRKKILITDLTPRDGQQCKLATRVRTDDLLPLCEAMDKVGFYAVEVWGGATYDVCLRYLKEDPWERLRRIKEVMPNTKLQMLFRGQNIVGYRPKSDKLVYKFVERAIKNGITVFRVFDALNDNRNIKTAVKAIKELGGEAHAEISYTRSPIHTYQKWIEYALEIAEMGADWLSFKDATGIIMPFETYAIIKGIKEATGGKLPVLLHNHDMSGTAIVNHMMAVLAGVDMLDTVLSPLAFGSSHPATESVVAMLEGTPFDTGIDMKKLDELAEIVKQIRKKYKKYETEYAGVNAKVLIHKIPGGMISNMVAQLIEANALDKIEEALEEVPNVERDLGHPPLLTPSSQIVGVQAVLNVISGERYKVITKEVRDYVEGKYGKPPGPISKELAEKILGPGKEPDFSIRAADLADPNDWDKAYEETKAILGREPTDEEVLLYALFPMQAKDFFVAREKGELHPEPVDELVETTEVKAGVVPGAAPVEFEIVYHGEKFKVKVEGVSAHQEPGKPRKYYIRVDGRLEEVQITPHVEAIPKGGPTPTAVQAEEKGIPKATQPGDATAPMPGRVVRVLVKEGDKVKEGQTVAIVEAMKMENEIHAPISGVVEKVFVKPGDNVTPDDALLRIKHIEEEVSYG

SEQ ID NO: 33 - DNA, 3401 bp, Hydrogenobacter thermophilus TK-6
atgtttaagaaggttttggtggcaaatagaggtgagatagcttgcagggttataagagcgtgtaaagagctgggtatacagacggttgccatatacaacgagattgaatccaccgcaaggcatgtaaagatggcggacgaagcctacatgataggtgtaaatcctctggatacctacctgaacgcagaaaggatagtggacctggctcttgaggtgggggctgaggctatacatcccggttatggctttctggcggagaacgagcactttgccagattgtgcgaagagaagggcataaccttcataggtccccactggaaggtcatagagcttatgggagacaaagccaggtcaaaggaggttatgaaaagggcgggcgtcccaacagtccctggaagcgacggcatactgaaagatgtagaagaagccaaacgcatagccaaagagataggctatcctgtgcttttaaaggcttctgcgggaggtggaggaaggggtataaggatatgcaggaacgaggaggagctggtaagaaactacgagaacgcttacaacgaggcggtcaaagccttcggcaggggggacctgcttctggaaaagtacatagaaaaccccaagcacattgagtttcaggttttgggagataagtacggaaatgttatacacctgggagaaagagactgttccatacagagaagaaaccaaaaacttgtagagattgccccatcgctccttcttacacctgagcaaagagagtattacggctctcttgtggtaaaagcagcaaaggagataggttattacagcgctggaactatggagtttatagccgacgaaaagggcaacctgtacttcatagagatgaacacccgcattcaggtggagcatccggtaactgagatgatcacaggtgttgacatagtaaagtggcaaatcaggatagcggcaggagaaaggttaaggtactctcaggaagacataaggttcaacggctactccatagagtgcaggataaacgcggaagatcccaagaaaggctttgcccccagcataggcaccatagagagatactatgtgcccggaggatttggcataagggttgaacatgcctcatcaaagggttacgagatcactccctattacgactccctcatagccaagctcatcgtgtgggctcctctctgggaggttgccgttgacagaatgaggtccgctcttgaaacctatgagatctccggtgtcaaaaccaccataccgctccttataaacatcatgaaggacaaagacttcagagatggtaaatttaccacaaggtacctggaggagcatccccatgtttttgattacgctgaacacagagacaaagaggactttgtagcttttatatccgcagtaatagccagttatcatgggctttgataaaaactctggaggtgtagtacatgcaagcagttgagattatggaagagataagagaaaagttcaaagagtttgaaaagggaggctttaggaagaaaatactcataacagaccttacgcccagggacggacagcagtgcaaactggcaactcgggtcagaacagatgaccttttgcccctctgtgaggctatggacaaggtggggttctatgcagttgaggtgtggggaggtgccacctatgatgtgtgcctcagatacctcaaagaagacccgtgggagagactgaggcgcataaaggaagtgatgcccaacactaagctccagatgctctttagaggtcaaaacatagtgggatacagacccaagtccgataagctggtttataagtttgtggagagagcaataaaaaacggtataaccgttttcagagtgtttgacgctctcaatgacaacaggaacataaagaccgctgtaaaagccataaaggagctgggtggtgaggcacatgccgagataagctatacaagaagtcctatacacacctaccaaaagtggatagagtacgctcttgaaatagcggagatgggtgcggactggctgtcttttaaggatgccacgggtataattatgccctttgaaacttacgccataataaagggtataaaggaagccacaggtggaaaacttccggtgctacttcacaaccacgacatgagcggaaccgccatagtcaatcacatgatggctgtgctggctggtgtggacatgctggatactgttctctcaccgcttgcctttggctcttctcaccctgcgacggaatccgtggttgccatgcttgaaggaacaccctttgatacaggcatagacatgaagaagttggacgaacttgctgagatagtaaagcaaataaggaagaagtacaaaaagtatgagacggagtatgctggtgtaaatgccaaagtgctcatccacaagatacccggcggtatgatatccaacatggtggcacagctcatagaggcaaatgctttggataaaatagaggaagctctcgaagaggtaccaaatgtggaaagggacctggggcatccaccacttctgacaccttcttcacagatagtgggtgttcaggcggttctcaatgtgatatccggtgagcgatacaaggttataaccaaagaggtaagagactatgtggaaggcaagtatggaaagccacccggtcccatatctaaggagctggctgagaagatcctcggtcctggaaaggaacccgacttctccataagagctgcagacctggcagaccccaacgactgggataaggcttatgaagagacaaaagctatacttggaagagagcctacagatgaggaggtgctcctttatgctctcttccccatgcaggcaaaggacttttttgtagccagagagaagggagaacttcatcctgagccagttgatgagcttgttgaaacaactgaagtaaaggcaggtgttgttccaggtgcagcacctgttgagtttgaaatcgtctatcacggtgagaagttcaaagtaaaagtggaaggtgtgagcgctcatcaggagcccggaaagcccagaaagtactacataagggtggacgggaggctggaagaggtgcagataacgccccatgtggaagctataccaaaaggaggacccactccaacggcagtacaagccgaagagaaaggcatacctaaagctacccagccaggtgatgctactgctcctatgccgggaagggtcgtgagggttttggtaaaggaaggtgataaagtaaaagaaggtcaaacggtagccatagtggaagccatgaagatggagaacgagatccacgctcccataagcggtgtagtagaaaaggtctttgtcaaacccggagataatgtaacacctgacgatgccctcctaagaataaaacacatagaggaagaggtcagttacggctga

SEQ ID NO: 34 - protein, 472 aa, Candidatus Nitrospira defluvii
MFRKILIANRGEIAMRIIRGCRELNIATAAIYSEADSSGIYVKKADESYLVGPGPVKGFLDGKQIVEIAKRIGADAIHPGYGFLSENTKFARLCQTSGITFIGPSPETIDLMGSKVKARQIAQQAGVPIVPGTEGGVTSVDDALAFAHQINYPVMIKASAGGGGRGLRVVRSDQELRENIDVASREAQAAFGDGSIFIEKYIERPHHIEFQILGDKHGNIIHLGERDCSIQRRHQKLIEIAPSLILTPKLRAQMGEAAIAIAKAVHYDNAGTVEFLLDHEGHFYFMEMNPRLQVEHTVTEQITAIDIVRNQISIAAGKPLEIRQKDVTLQGHAIQCRINAEDPRNNFMPCTGTITAYLSPGGIGVRIDGAVYRDYTIPPYYDALLAKLTVRGRTWEETVSRMRRSLEEYVLRGVKTTIPFMKNVMMEQDFQAGRFDTSYLETHPDLYQYEESEEPEDLVLAISAAIAAYEGL

SEQ ID NO: 35 - protein, 643 aa, Candidatus Nitrospira defluvii
MRVKPSRPSASRAVQVMQAASPEFRVTPAPGKKLLMTEVALRDGHQCLLATRMRTEDMLPIAQKLDAVGFWSLEVWGGATFDTCLRFLKEDPWERLRALRAAMPKTKLQMLLRGQNLVGYRHYADDVLEKFIERSAFNGIDVFRIFDALNDVRNLERAIREVKACEKHVEAAISYTTSPVHRLDGFVTMGKRLEDLGADTICIKDMAGLLAPVDAYRLVKSLKAAVRVPIHLHSHYTSGMGTMSALMAVMAGLDLLDTSISPLAGGASHPPTESMVAALRGTPYDSGLDLEDLQPIAEHFRNVRRKYRQFESDFTGVDAEILTSQIPGGMLSNLAAQLAEQNALDRMKEVMDEIPRVRKDMGYPPLVTPTSQIVGTQATLNVLTGEQGERYKVITTETKNYFLGLYGRAPGPLDKEIMARAIGDEEPVKGRPADRLESEFEKLKKDMPESATTLEDQLSFALFPAIARDFFEARERGDLRAEPLEPTETKGPAVAHDLHLAPAEFNITVHGENYHVVVSGSGRTTDGRKPYYIRVNDRLQEVSLEPLQEVLAGVPESPEAGSTSKPKRPRPTKPGDVAPPMPGRVVKVLVTDGAQVKTGDPLLIIEAMKMESQVPAPMDGRVAAILVVEGDNVKIDETVIQLE

SEQ ID NO: 36 - DNA, 3374 bp, Candidatus Nitrospira defluvii
atgtttcggaagatccttattgccaaccgtggcgaaatcgccatgcgcatcatccgtggctgtcgtgagctcaatatcgcgacagcggcgatctattctgaagccgactcttcaggaatctacgtcaaaaaagccgacgagtcctacctcgtaggcccgggacccgtcaaggggttcctggacggaaaacagatcgtggagatcgccaagcgcatcggcgccgacgcgattcatcccggatacgggttcctctctgaaaacactaaattcgcccggctctgccaaacctcaggcattaccttcatcggtccgtcccccgagacgatcgacctcatgggcagcaaagtgaaggcgcgacagatcgcccagcaggcgggggtcccgatcgtccccggcaccgaaggcggagtcaccagcgtcgacgacgccctggccttcgcccatcagatcaactaccccgtcatgatcaaggccagcgccggcggcgggggccgaggattgcgggtcgtccggtccgatcaggaattgcgagagaacatcgatgtcgcgtcgcgagaagcacaggccgcgttcggcgacggcagcatcttcatcgagaaatacatcgaacgaccgcaccatatcgaatttcaaatcctgggcgacaaacacggcaacatcatccacctgggtgagcgggattgttccattcaacggcggcaccagaaactgatcgaaatcgccccctcattgatcctgacgcccaaactgcgcgcccaaatgggcgaggccgccattgccatcgcgaaagcggtgcactacgacaatgccggcaccgtcgagttcctcctcgaccacgagggccatttctacttcatggaaatgaatccccgcctccaggtggaacataccgtcacggaacagatcacggccatcgatatcgtccgcaatcaaatttccattgcggcgggaaagcctctggagatccggcagaaggacgtaacgttgcagggccatgcgattcagtgccgcatcaatgccgaagacccgcgcaacaacttcatgccctgcacaggcaccatcaccgcctatctgtcacccggcggaatcggagtccgcatcgacggcgcggtctatcgcgattacacgattcctccctattatgatgcgctgttggcaaaactgaccgtccgcgggcgcacctgggaagagaccgtgagccgcatgcggcgttcccttgaagagtatgtgctgcgcggggtgaaaacgaccattccgttcatgaagaacgtgatgatggaacaggattttcaagccggacgattcgatacgtcctacctggaaacccatccggacctgtatcaatacgaagaatccgaggagcctgaggacctggtgctggccatctccgcagcgatcgccgcgtacgaaggactctgataaaaactctggaggtgtagtacatgcgtgtaaaacccagccggccctctgcctcacgcgccgtccaggttatgcaggcggcgagccctgagttccgcgtgaccccggcgccggggaaaaagcttttaatgaccgaggttgcgttgcgcgacgggcatcaatgcctactcgcgaccaggatgcgcaccgaggacatgctacccatcgcccaaaaactggacgctgtgggattctggtcgttggaagtctggggcggcgccaccttcgatacctgcctccggttcctcaaggaagacccctgggagcgcctgcgcgcgctccgcgcggcgatgccgaagacgaagctgcaaatgttgttgcgcggccagaacctggtcgggtatcgccactacgccgacgacgtgctggagaagtttatcgagcgctcggcgtttaacggcatcgatgtcttccgcatcttcgacgccctcaacgatgttcgcaatctggagcgggccatccgtgaagtgaaagcctgcgaaaagcatgtggaagcggccatctcctacaccaccagcccggtccaccggctggacgggttcgtcacgatgggcaaacggttggaagacctgggcgccgataccatctgcatcaaagacatggccggcctgctggcgcccgtcgatgcctaccgtctggtcaagagcctcaaagcagcggttcgcgtgcccatccacctgcactcccactacacctcgggcatgggaaccatgtcggcgctgatggcggtcatggccgggctcgatctcctggacacctcgatttctccgcttgccggaggcgcctcgcatccccccaccgaatctatggtggctgcgttacggggcacgccctatgacagcggattggacctggaagatctgcagcccattgcagagcatttccgaaacgtgcgccggaagtaccggcaatttgaaagcgacttcaccggtgtggacgctgaaattctgacgtcccagattcccggcggcatgctctccaatctcgccgcccaactggccgaacaaaacgccttggaccgaatgaaagaagtgatggacgaaattccccgtgtccgcaaagacatgggctatccgccgcttgtcacgccgaccagccagatcgtcggcacgcaggccaccctcaacgtgctcactggtgaacagggcgagcgctacaaggtcatcactacggagaccaagaattatttcctcggcctctacggccgggctcccgggccgcttgataaagagatcatggcacgggccatcggggacgaagagcccgtaaagggccgaccggccgaccggcttgaatcggaatttgaaaaactcaagaaggacatgcccgagtccgccacgacgctggaagatcaactgtcgttcgccctcttccccgcgattgccagggatttcttcgaagcacgcgagcggggcgacctgcgggcagagccgctggagccgacggaaacgaagggtcctgccgtggcccacgatctccacctcgcgccggccgaattcaacatcaccgtgcacggcgagaattatcatgtcgtggtctcgggctcaggccgcaccaccgacggccgcaagccttactacatccgggtcaacgaccggctgcaggaagtctcactggaaccgctgcaggaagtgctggccggcgtgcccgaatccccagaggccggcagcacgagcaagccgaaacggccccgaccgaccaaacccggcgatgtcgccccgcccatgcccggtcgtgtcgtgaaagtcctggtaacggacggcgcccaggtaaagaccggtgatccgctcctgatcattgaggccatgaaaatggaaagccaagttcctgcgccgatggacgggcgggtcgcggcgattctggtcgtcgaaggcgacaacgtcaagatcgacgaaaccgtcattcaactggagtag

SEQ ID NO: 37 - protein, 472 aa, Hydrogenobacter thermophilus TK-6; identical to SEQ ID NO: 31

SEQ ID NO: 38 - protein, 652 aa, Hydrogenobacter thermophilus TK-6
MQAVEIMEEIREKFKEFEKGGFRKKILITDLTPRDGQQCKLATRVRTDDLLPLCEAMDKVGFYAVEVWGGATYDVCLRYLKEDPWERLRRIKEVMPNTKLQMLFRGQNIVGYRPKSDKLVYKFVERAIKNGITVFRVFDALNDNRNIKTAVKAIKELGGEAHAEISYTRSPIHTYQKWIEYALEIAEMGADWLSFKDATGIIAPVETYAIIKGIKEATGGKLPVLLHNHDMSGMATVNHLMAVLAGVDMLDTVLSPLAFGSSHPATESVVAMLRGTPFDTGIDMKKLQELAEIVKQIRKKYKKYETEYAGVNAKVLIHKIPGGMISNMVAQLIEANALDKIEEALEEVPNVERDLGHPPLLTPSSQIVGVQAVLNVISGERYKVITKEVRDYVEGKYGKPPGPISKELAEKILGPGKEPDFSIRAADLADPNDWDKAYEETKAILGREPTDEEVLLYALFPMQAKDFFVAREKGELHPEPVDELVETTEVKAGVVPGAAPVEFEIVYHGEKFKVKVEGVSAHQEPGKPRKYYIRVDGRLEEVQITPHVEAIPKGGPTPTAVQAEEKGIPKATQPGDATAPMPGRVVRVLVKEGDKVKEGQTVAIVEAMKMENEIHAPISGVVEKVFVKPGDNVTPDDALLRIKHIEEEVSYG

SEQ ID NO: 39 - DNA, 3401 bp, Hydrogenobacter thermophilus TK-6
atgtttaagaaggttttggtggcaaatagaggtgagatagcttgcagggttataagagcgtgtaaagagctgggtatacagacggttgccatatacaacgagattgaatccaccgcaaggcatgtaaagatggcggacgaagcctacatgataggtgtaaatcctctggatacctacctgaacgcagaaaggatagtggacctggctcttgaggtgggggctgaggctatacatcccggttatggctttctggcggagaacgagcactttgccagattgtgcgaagagaagggcataaccttcataggtccccactggaaggtcatagagcttatgggagacaaagccaggtcaaaggaggttatgaaaagggcgggcgtcccaacagtccctggaagcgacggcatactgaaagatgtagaagaagccaaacgcatagccaaagagataggctatcctgtgcttttaaaggcttctgcgggaggtggaggaaggggtataaggatatgcaggaacgaggaggagctggtaagaaactacgagaacgcttacaacgaggcggtcaaagccttcggcaggggggacctgcttctggaaaagtacatagaaaaccccaagcacattgagtttcaggttttgggagataagtacggaaatgttatacacctgggagaaagagactgttccatacagagaagaaaccaaaaacttgtagagattgccccatcgctccttcttacacctgagcaaagagagtattacggctctcttgtggtaaaagcagcaaaggagataggttattacagcgctggaactatggagtttatagccgacgaaaagggcaacctgtacttcatagagatgaacacccgcattcaggtggagcatccggtaactgagatgatcacaggtgttgacatagtaaagtggcaaatcaggatagcggcaggagaaaggttaaggtactctcaggaagacataaggttcaacggctactccatagagtgcaggataaacgcggaagatcccaagaaaggctttgcccccagcataggcaccatagagagatactatgtgcccggaggatttggcataagggttgaacatgcctcatcaaagggttacgagatcactccctattacgactccctcatagccaagctcatcgtgtgggctcctctctgggaggttgccgttgacagaatgaggtccgctcttgaaacctatgagatctccggtgtcaaaaccaccataccgctccttataaacatcatgaaggacaaagacttcagagatggtaaatttaccacaaggtacctggaggagcatccccatgtttttgattacgctgaacacagagacaaagaggactttgtagcttttatatccgcagtaatagccagttatcatgggctttgataaaaactctggaggtgtagtacatgcaagcagttgagattatggaagagataagagaaaagttcaaagagtttgaaaagggaggctttaggaagaaaatactcataacagaccttacgcccagggacggacagcagtgcaaactggcaactcgggtcagaacagatgaccttttgcccctctgtgaggctatggacaaggtggggttctatgcagttgaggtgtggggaggtgccacctatgatgtgtgcctcagatacctcaaagaagacccgtgggagagactgaggcgcataaaggaagtgatgcccaacactaagctccagatgctctttagaggtcaaaacatagtgggatacagacccaagtccgataagctggtttataagtttgtggagagagcaataaaaaacggtataaccgttttcagagtgtttgacgctctcaatgacaacaggaacataaagaccgctgtaaaagccataaaggagctgggtggtgaggcacatgccgagataagctatacaagaagtcctatacacacctaccaaaagtggatagagtacgctcttgaaatagcggagatgggtgcggactggctgtcttttaaggatgccacgggtataattgcgcccgtcgaaacttacgccataataaagggtataaaggaagccacaggtggaaaacttccggtgctacttcacaaccacgacatgagcggaatggccaccgtcaatcacctgatggctgtgctggctggtgtggacatgctggatactgttctctcaccgcttgcctttggctcttctcaccctgcgacggaatccgtggttgccatgcttcggggaacaccctttgatacaggcatagacatgaagaagttgcaggaacttgctgagatagtaaagcaaataaggaagaagtacaaaaagtatgagacggagtatgctggtgtaaatgccaaagtgctcatccacaagatacccggcggtatgatatccaacatggtggcacagctcatagaggcaaatgctttggataaaatagaggaagctctcgaagaggtaccaaatgtggaaagggacctggggcatccaccacttctgacaccttcttcacagatagtgggtgttcaggcggttctcaatgtgatatccggtgagcgatacaaggttataaccaaagaggtaagagactatgtggaaggcaagtatggaaagccacccggtcccatatctaaggagctggctgagaagatcctcggtcctggaaaggaacccgacttctccataagagctgcagacctggcagaccccaacgactgggataaggcttatgaagagacaaaagctatacttggaagagagcctacagatgaggaggtgctcctttatgctctcttccccatgcaggcaaaggacttttttgtagccagagagaagggagaacttcatcctgagccagttgatgagcttgttgaaacaactgaagtaaaggcaggtgttgttccaggtgcagcacctgttgagtttgaaatcgtctatcacggtgagaagttcaaagtaaaagtggaaggtgtgagcgctcatcaggagcccggaaagcccagaaagtactacataagggtggacgggaggctggaagaggtgcagataacgccccatgtggaagctataccaaaaggaggacccactccaacggcagtacaagccgaagagaaaggcatacctaaagctacccagccaggtgatgctactgctcctatgccgggaagggtcgtgagggttttggtaaaggaaggtgataaagtaaaagaaggtcaaacggtagccatagtggaagccatgaagatggagaacgagatccacgctcccataagcggtgtagtagaaaaggtctttgtcaaacccggagataatgtaacacctgacgatgccctcctaagaataaaacacatagaggaagaggtcagttacggctaa

SEQ ID NO: 40 - protein, 472 aa, Hydrogenobacter thermophilus TK-6; identical to SEQ ID NO: 31

SEQ ID NO: 41 - protein, 652 aa, Hydrogenobacter thermophilus TK-6
MQAVEIMEEIREKFKEFEKGGFRKKILITDLTPRDGQQCKLATRVRTDDLLPLCEAMDKVGFYAVEVWGGATYDVCLRYLKEDPWERLRRIKEVMPNTKLQMLFRGQNIVGYRPKSDKLVYKFVERAIKNGITVFRVFDALNDNRNIKTAVKAIKELGGEAHAEISYTRSPIHTYQKWIEYALEIAEMGADWLSFKDATGIIAPVETYAIIKGIKEATGGKLPVLLHNHDMSGMAIVNHLMAVLAGVDMLDTVLSPLAFGSSHPATESVVAMLEGTPFDTGIDMKKLDELAEIVKQIRKKYKKYETEYAGVNAKVLIHKIPGGMISNMVAQLIEANALDKIEEALEEVPNVERDLGHPPLLTPSSQIVGVQAVLNVISGERYKVITKEVRDYVEGKYGKPPGPISKELAEKILGPGKEPDFSIRAADLADPNDWDKAYEETKAILGREPTDEEVLLYALFPMQAKDFFVAREKGELHPEPVDELVETTEVKAGVVPGAAPVEFEIVYHGEKFKVKVEGVSAHQEPGKPRKYYIRVDGRLEEVQITPHVEAIPKGGPTPTAVQAEEKGIPKATQPGDATAPMPGRVVRVLVKEGDKVKEGQTVAIVEAMKMENEIHAPISGVVEKVFVKPGDNVTPDDALLRIKHIEEEVSYG

SEQ ID NO: 42 - DNA, 3401 bp, Hydrogenobacter thermophilus TK-6
atgtttaagaaggttttggtggcaaatagaggtgagatagcttgcagggttataagagcgtgtaaagagctgggtatacagacggttgccatatacaacgagattgaatccaccgcaaggcatgtaaagatggcggacgaagcctacatgataggtgtaaatcctctggatacctacctgaacgcagaaaggatagtggacctggctcttgaggtgggggctgaggctatacatcccggttatggctttctggcggagaacgagcactttgccagattgtgcgaagagaagggcataaccttcataggtccccactggaaggtcatagagcttatgggagacaaagccaggtcaaaggaggttatgaaaagggcgggcgtcccaacagtccctggaagcgacggcatactgaaagatgtagaagaagccaaacgcatagccaaagagataggctatcctgtgcttttaaaggcttctgcgggaggtggaggaaggggtataaggatatgcaggaacgaggaggagctggtaagaaactacgagaacgcttacaacgaggcggtcaaagccttcggcaggggggacctgcttctggaaaagtacatagaaaaccccaagcacattgagtttcaggttttgggagataagtacggaaatgttatacacctgggagaaagagactgttccatacagagaagaaaccaaaaacttgtagagattgccccatcgctccttcttacacctgagcaaagagagtattacggctctcttgtggtaaaagcagcaaaggagataggttattacagcgctggaactatggagtttatagccgacgaaaagggcaacctgtacttcatagagatgaacacccgcattcaggtggagcatccggtaactgagatgatcacaggtgttgacatagtaaagtggcaaatcaggatagcggcaggagaaaggttaaggtactctcaggaagacataaggttcaacggctactccatagagtgcaggataaacgcggaagatcccaagaaaggctttgcccccagcataggcaccatagagagatactatgtgcccggaggatttggcataagggttgaacatgcctcatcaaagggttacgagatcactccctattacgactccctcatagccaagctcatcgtgtgggctcctctctgggaggttgccgttgacagaatgaggtccgctcttgaaacctatgagatctccggtgtcaaaaccaccataccgctccttataaacatcatgaaggacaaagacttcagagatggtaaatttaccacaaggtacctggaggagcatccccatgtttttgattacgctgaacacagagacaaagaggactttgtagcttttatatccgcagtaatagccagttatcatgggctttgataaaaactctggaggtgtagtacatgcaagcagttgagattatggaagagataagagaaaagttcaaagagtttgaaaagggaggctttaggaagaaaatactcataacagaccttacgcccagggacggacagcagtgcaaactggcaactcgggtcagaacagatgaccttttgcccctctgtgaggctatggacaaggtggggttctatgcagttgaggtgtggggaggtgccacctatgatgtgtgcctcagatacctcaaagaagacccgtgggagagactgaggcgcataaaggaagtgatgcccaacactaagctccagatgctctttagaggtcaaaacatagtgggatacagacccaagtccgataagctggtttataagtttgtggagagagcaataaaaaacggtataaccgttttcagagtgtttgacgctctcaatgacaacaggaacataaagaccgctgtaaaagccataaaggagctgggtggtgaggcacatgccgagataagctatacaagaagtcctatacacacctaccaaaagtggatagagtacgctcttgaaatagcggagatgggtgcggactggctgtcttttaaggatgccacgggtataattgcgcccgtcgaaacttacgccataataaagggtataaaggaagccacaggtggaaaacttccggtgctacttcacaaccacgacatgagcggaatggccatagtcaatcacctgatggctgtgctggctggtgtggacatgctggatactgttctctcaccgcttgcctttggctcttctcaccctgcgacggaatccgtggttgccatgcttgaaggaacaccctttgatacaggcatagacatgaagaagttggacgaacttgctgagatagtaaagcaaataaggaagaagtacaaaaagtatgagacggagtatgctggtgtaaatgccaaagtgctcatccacaagatacccggcggtatgatatccaacatggtggcacagctcatagaggcaaatgctttggataaaatagaggaagctctcgaagaggtaccaaatgtggaaagggacctggggcatccaccacttctgacaccttcttcacagatagtgggtgttcaggcggttctcaatgtgatatccggtgagcgatacaaggttataaccaaagaggtaagagactatgtggaaggcaagtatggaaagccacccggtcccatatctaaggagctggctgagaagatcctcggtcctggaaaggaacccgacttctccataagagctgcagacctggcagaccccaacgactgggataaggcttatgaagagacaaaagctatacttggaagagagcctacagatgaggaggtgctcctttatgctctcttccccatgcaggcaaaggacttttttgtagccagagagaagggagaacttcatcctgagccagttgatgagcttgttgaaacaactgaagtaaaggcaggtgttgttccaggtgcagcacctgttgagtttgaaatcgtctatcacggtgagaagttcaaagtaaaagtggaaggtgtgagcgctcatcaggagcccggaaagcccagaaagtactacataagggtggacgggaggctggaagaggtgcagataacgccccatgtggaagctataccaaaaggaggacccactccaacggcagtacaagccgaagagaaaggcatacctaaagctacccagccaggtgatgctactgctcctatgccgggaagggtcgtgagggttttggtaaaggaaggtgataaagtaaaagaaggtcaaacggtagccatagtggaagccatgaagatggagaacgagatccacgctcccataagcggtgtagtagaaaaggtctttgtcaaacccggagataatgtaacacctgacgatgccctcctaagaataaaacacatagaggaagaggtcagttacggctga

SEQ ID NO: 43 - protein, 472 aa, Thiocystis violascens DSM198
MLRKILIANRGEIAVRVIRACAEMGIRSAAIYAEADRHSLHVKKADEAYSLGSDPLAGYLNVHNIVNLALSTGCDAVHPGYGFLSENPELARACARRGLTFIGPTAEVIARMGDKTEARLAMQKAGVPVTPGSPGNLESLDAALRFADEIGYPIMLKATSGGGGRGIRRCDDAHALRNNYERVISEATKAFGRAEVFLEKCVVNPKHIEVQILGDHHGNCVHLYERDCSIQRRNQKLIEIAPSPQLDEAERQYVGGLAVLAARAVGYTNAGTIEFLRDSDGRFYFMEMNTRIQVEHTITETITGVDLVEEQIRIAAGLPLRFKQHEIQRRGFAMQFRVNAEDPKNNFLPSFGRISRYYAPGGPGVRTDGAIYTGYTVPPHYDSMLAKVIVWALNWEDVVNRGHRALRDIGVYGVKTTIPFYQEILRHPDFRSGSFDTSFLETHPELLDYSTKRRREDVAAVLAAAIAAHAGL

SEQ ID NO: 44 - protein, 609 aa, Thiocystis violascens DSM198
MPKINITDVVLRDAHQSLLATRMRTEDMLPICPKLDAIGYWSLECWGGATFDACVRFLKEDPWERLRKLREALPNTRLQMLLRGQNLLGYRHYSDDVVRAFVARAAQNGMDVFRIFDALNDPRNLKTAIEATKAAGKHAQGTICYTVSPVHTVAGFVQLGKELAAMGCDSIAIKDMAGLLTPYVTAELVKALKDSVDLPLHLHSHATSGLADMCHLKAIENGCDTLDTAISSMAGGTSHPPTESLVAALRGTDYDTGLDLEAIQEVGMYFYQIRKKYHQFESDFTGVDTRVQVNQVPGGMISNLANQLKEQNSLERMNAVLEEIPRVRMDLGYPPLVTPTSQIVGTQAVLNVLTDKRYQTITNEVKLYLQGRYGRAPGAINPTLQQQAIGNEDLIDCRPADLLTPEMERLRHDIGELAISEEDALTYAMFPEIGRAFLEHRAAGTLHPEPLEPLPSGAGPRTAPTEFNIAVHGETYHVKVTGTGHKSQDERHFYFAIDGIPEEVVVETLDELVLTGGAQGAVKKAIAGKRPKPTQPGHVATSMPGNIVDVLVKEGDTVAAGQPVLITEAMKMETEIQAPIAGTVTAMFVIKGDAVNPDEVLLEITPAER

SEQ ID NO: 45 - DNA, 3272 bp, Thiocystis violascens DSM198
atgcttcgaaagattctgatcgcgaaccgcggcgagattgcggtccgtgtcatccgcgcctgtgccgagatggggatccgctcggcggccatctatgccgaggccgaccgtcattcgctccatgtcaaaaaggccgacgaagcctatagcctgggcagcgatccgctggcgggctatctcaatgtccacaacatcgtcaacctggccctgtcgaccggttgcgatgccgtgcatcccggctacggttttctgtccgaaaacccggaactggcgcgcgcctgcgcgcgacgcggactgaccttcatcggcccgaccgccgaggtgatcgcccgcatgggcgacaagaccgaggcgcggctcgcgatgcagaaggccggtgttccggtgacgcccggcagccccggcaacctggagagcctggacgcggccctgcgcttcgccgacgagatcggctatccgatcatgctcaaggcgacctccggcggcggcgggcgcggcatccggcgctgtgacgatgcccatgcgctgcgcaataactacgagcgcgtcatctccgaagccaccaaggcgtttggtcgcgccgaggtcttcctggaaaagtgcgtggtcaatcccaaacacatcgaagttcagatcttgggcgatcatcatggcaactgcgtgcatctctacgagcgcgattgctcgatccagcgacgcaatcagaagctgatcgagatcgccccctcgccgcagctcgacgaggccgaacgccagtatgtcggcggcctggcggtgctggcggcgcgcgctgtcggttacaccaatgccggcaccatcgagtttctgcgcgattcggacgggcgtttctatttcatggagatgaacacccgcatccaggtcgagcacaccatcaccgagaccatcaccggggtcgatctggtggaggaacagatccgcattgccgccgggctgccgctgcgtttcaagcagcacgagatccaacggcgcggcttcgccatgcagttccgcgtcaatgccgaggatcccaagaacaatttcctgccgagcttcgggcgcatctcgcgctattacgcccccggcggtccgggcgtgcgtaccgatggggcgatctacaccggctacacggttccgccgcattatgattccatgctggccaaggtgatcgtctgggcgctgaactgggaggatgtcgtcaatcgcggccatcgcgcgctgcgcgacatcggcgtctatggcgtcaagaccaccatccccttctatcaggagatcctgcgtcaccccgattttcgctctggatccttcgataccagttttctggagacgcatcccgagttgctggactattccaccaaacgtcgccgcgaggatgtcgccgccgtgctggcagcggcgatcgcggcgcatgccggtttgtaataaaaactctggaggtgtagtacatgccaaagatcaacattaccgacgttgtcctgcgcgacgcccaccagtcgctgctcgcgacgcgcatgcgcaccgaggacatgctgccgatctgtcccaagctggacgccatcggctactggtcgctggaatgctggggcggcgcgaccttcgatgcctgcgtgcgcttcctgaaggaagatccctgggagcgtctgcgcaagctgcgcgaggcgctgccgaacactcgcctgcagatgctgctgcgcggccagaatctgcttggctaccgtcattattccgatgacgtggtacgcgccttcgtggcccgtgctgcccagaacggcatggatgtgttccgcattttcgatgcactcaacgatccgcgcaatctcaagacggcgatcgaggccaccaaggccgccggcaagcatgcccaaggcaccatctgctacacggtcagtccggttcacaccgtggccggtttcgtccagttggggaaggaactggcggccatgggctgcgactccatcgccatcaaggacatggcgggtctgctgacgccctatgtcacggccgagctggtgaaggcgctgaaggatagcgtcgacctgccgctgcatctgcactcgcacgccacctcaggtctggccgatatgtgccatctgaaggccatcgagaacggctgtgataccctggataccgccatttcatcgatggctggcggcacctcgcacccgcccaccgagagtctggtcgccgcattgcgcggcaccgactacgacaccggcctggacctggaggcgatccaggaagtcgggatgtatttctatcagatccgcaagaagtaccaccagttcgagagcgacttcaccggcgtggacacccgggtccaggtcaatcaagtgcccggcggcatgatctccaatctggccaaccagttgaaggaacagaattcgctggagcgcatgaacgcggtgctcgaagagattccgcgagtacgcatggatctcggctatcccccgctggtgacgccaacctcgcagatcgtcggcacccaggcggtgctcaacgtcctgaccgacaagcgctaccagaccatcaccaacgaggtgaagctctatctgcaggggcgctacggacgcgcgccgggcgcgattaacccgacccttcagcagcaggccatcggcaacgaggacctgatcgactgccgcccggccgacctgctgacaccggagatggagcgactccgccacgatatcggcgaactcgcaatctccgaggaagacgccctcacgtatgccatgttcccggagatcgggcgcgctttcctggaacatcgcgccgccggcaccctgcatccggaaccgctggagccgctacccagcggcgctggcccccgcaccgcgcccaccgagttcaatatcgccgtccatggcgagacctatcacgtcaaagtgacaggcacgggacataagagtcaggacgaacgtcatttctatttcgccatcgatggcatcccggaagaggtggtggtcgagacgctcgacgaactggtgctgacgggcggcgcccagggcgcggtcaagaaagccatcgccggcaagcgtcccaagcccactcagcccggccatgtcgccacctcgatgcccggcaacatcgtcgacgtgctggtgaaggaaggcgatacggtggcggccggtcagccggtgctgatcaccgaggcgatgaagatggagaccgagattcaggcgcccatcgccgggacggtcaccgccatgttcgtcatcaagggcgatgcggtgaatccggatgaggtgttgctggagatcacgccggctgagcgttaa

SEQ ID NO: 46 - protein, 472 aa, Mariprofundus ferrooxydans PV-1
MFKRILVANRGECAIRIIRSCRELGIESVAIYSEADAHALHVKKADRAVMIGPDPVKSYLNIHRIVGVALDSGCDAVHPGYGFLSENDEFARAIIDAGLTYIGPSPDAIRDMGSKTKARESMIAAGVPVIPGSDGALNNVDEALELAHKMGYPVMLKAAAGGGGRGIRRCDSDAQLRENYVVTQREAMAAFGSDILFMEKCIVEPHHIEFQVLADSHGNTVHLFERDCSIQRRNQKLIEIAPSNFLTPKLRESMGAIAVKAAQAVGYVNAGTVEFLVDKDRNFWFMEMNTRLQVEHTITETITGVDIVAQQISIAAGEALPFTQADLSFRGFAIEFRINAEDPKNNFLPMPGRITRYISPGGMGVRVDGCVYAGYEIPPYYDSMCAKLTVSGLNWHNTVMRAQRALGEYDIRGMKTTLPFYRTIASSEVFMQGEFNTGFMDQHPELLDYNDNERREDIAAAVAMAIAVHAGL

SEQ ID NO: 47 - protein, 617 aa, Mariprofundus ferrooxydans PV-1
MTDTKKKLAITELALRDGHQSLLATRMRLDDMLPICEKLDTIGYWSIEAWGGATFDTCLRYLKEGPWVRLRELNKALPNTPIQMLLRGQNLLGYRHYADDVVKKFVDMAAANGVDVFRVFDAMNDLRNVRTAVNQVKANDKHAEGTICYTTSPVHTLEYFIDLGKGFEDMGCDTLAIKDMAGLLTPTATRELILALKQSVSIPLHLHSHATAGVAEMVQWEAVHAGCDIIDTAISPLAGGTSHPPTEAMVAAFAGTEYDTGLNLVALQEIAAYFKEVRKKYARFESDSTGVDTRVFVNQIPGGMISNLANQLRDQGAQDKMDAVLDEIPRVRKDFGYPPLVTPTSQIVGTQAVLNVMSGKKYKVITNETRDYLKGLYGRALGEINEEVRKLAIGDEEPIDIRPADLLVPELDALTREVGDRATSVEDVLSYALFPTIALEFFEERASGQFKPESLDTPLEASSTPEVVTAPSLAPTEFNIIIHGEEYHIKIEGSGHKSDDVRPFYVKVDNVLEEVTVETLTEVVPTHNGNFDVSKASKGSRRPKATSDSDVTTAMPGRIVAINVAIGDQVEAGTTVLTVEAMKMENQVHAPVSGTVTAINVAVGDSVNPDECLMQID

SEQ ID NO: 48 - DNA, 3362 bp, Mariprofundus ferrooxydans PV-1
atgtttaaacgtattctggtagccaaccgtggtgagtgtgccattcgaattatccgttcatgtcgtgagctgggtatcgaatcggttgccatctattctgaagctgatgcccatgccctgcatgtgaaaaaagccgatcgcgctgtgatgatcggtcctgatccggtcaagagctatctgaacattcacaggatagtcggcgtcgcactggactccggttgcgatgctgtacatccgggctacggcttcctctctgaaaacgatgaatttgcgcgggcgattatcgatgcaggactgacctatatcggcccctcccccgacgcaatccgtgatatgggtagcaagaccaaggcacgcgaatcgatgattgccgccggcgttccggtgattcccggttcggacggagctctcaacaatgtcgatgaggcgctggagctggcgcataaaatgggttacccggtcatgctcaaggcggcggccggcggcggcggacgcggcattcgtcgctgcgacagcgatgctcaactgcgcgaaaattatgtcgtaacccagcgcgaagcgatggctgcattcggctccgatatcctgttcatggaaaaatgcattgtcgaaccgcatcatattgaattccaggttctggccgacagtcatggcaataccgtgcacctgtttgaacgcgactgctcaattcagcgacgtaaccagaagctgatcgaaattgccccgagcaactttctcacccccaagctgcgtgagagcatgggcgccattgcggtcaaggcagctcaggctgtgggctatgtcaatgccggtaccgtcgaatttctggtcgacaaggacagaaacttctggttcatggagatgaacacccgcctgcaggtggagcataccatcaccgaaaccattaccggcgtcgatattgtcgcccagcagatctcgattgcagcaggtgaagcccttcccttcacgcaggcggatctgagcttccgtggctttgccatcgagtttcgcatcaatgccgaagatccgaaaaacaacttcctgccgatgcccggtcgtattacccgctatatatctcccggcggcatgggtgtgcgcgtggatggctgcgtctatgccggctacgaaatcccgccctactacgattcgatgtgtgccaaactgacggtatccggtctgaactggcataacaccgtcatgcgggcccagcgtgcactcggcgaatacgatattcgcggcatgaaaaccacgctaccgttttaccgtactatcgcctcatcggaagtgttcatgcagggtgaattcaacaccggctttatggatcagcatccggagctgctggattacaacgataatgagcggcgtgaagatatcgctgctgcggtggcgatggccatcgccgtgcatgccggcctgtaatcgggtcgggaaggttaacgtcgctggcacgcccgtgtgccaacatgcggataagcaaacacaacatcgcgtaaaaaaggtatagagatatgactgacacaaagaaaaaactggcaattaccgaactggctctgcgtgacggacatcagtcgctgctggctacgcgtatgcggctcgacgacatgctgccgatttgcgagaagctcgatactatcggctactggtcgattgaagcgtggggcggcgcgaccttcgatacctgcctgcgctacctgaaagagggtccgtgggtacgcttgcgtgagctgaacaaggcgctgccgaacacacccatccagatgctgctgcgcggccagaacctgcttggctaccgtcattatgccgacgatgtggtgaagaagtttgtcgatatggctgccgccaacggcgttgacgtattccgtgtattcgatgcaatgaatgacctgcgcaatgtgcgtacggccgtgaatcaggtcaaagccaacgacaagcacgccgagggcaccatctgctacaccaccagcccggtacatacgctggaatactttatcgatctgggtaagggcttcgaagatatgggctgcgacacgctggcgatcaaggatatggcgggactgcttacgccgacggctacgcgtgaactgatcctggccctgaaacagtctgtctccatcccgctgcatctgcactcccacgcaacagccggcgtggccgagatggtacagtgggaagcggtgcatgccggttgcgacatcatcgataccgccatcagcccgctggccggcggcaccagccatccaccgacagaagccatggtcgcggcctttgccggtactgaatacgacacaggtctgaatctggtagcgttgcaggaaatcgccgcctacttcaaggaagtgcgtaaaaaatatgcccgttttgaatccgattcaaccggcgtggacacccgcgtattcgtcaaccagatccctggcggcatgatctccaatctggccaatcagctacgtgatcagggcgcacaggataagatggacgccgtgctcgatgaaattccacgcgtccgcaaggatttcggctacccgccactggtcacaccaaccagccagattgtcggcacccaggccgtgctcaatgtcatgtccggcaagaaatacaaggtcattaccaacgagacgcgcgactacctgaaaggcttgtatggccgtgcactcggcgaaatcaatgaagaggtgcgcaagctggccatcggcgatgaagagccgattgatatccgtcctgccgacctgctggtgcctgagctcgatgccctgacccgtgaagtcggtgatcgggctacttcggtggaggatgtactctcctatgccctgttcccgaccattgctctggagtttttcgaagagcgggccagcggtcagttcaaacctgaatcactggacacgcctctggaagccagttccacacctgaggttgttaccgcaccgtccctggcgcctaccgaattcaacatcatcattcatggtgaagaataccatatcaagatcgaaggttccggtcacaagagcgatgatgtgcgtccgttttatgtcaaggtggataatgtactggaagaggtcaccgttgagacgctgaccgaggtcgtacctacccataacggcaattttgatgtcagcaaggcatccaagggttcacgcaggccgaaagcaaccagcgacagcgatgtaacaacggccatgccgggtcgtatcgtggcgatcaatgtcgccatcggcgaccaggtagaagccggcaccaccgtcctgaccgtggaagcgatgaagatggaaaatcaggtgcatgcaccggtttccggtacggtcaccgccatcaatgtcgcagtcggcgatagcgtcaatcccgatgagtgcctgatgcagatcgactaa

SEQ ID NO: 49 - protein, 558 aa, Pseudomonas stutzeri ATCC14405
MRINDFRIVLPVVRLHFAEQSNLRRFCLTGQETVIPDTHISKYLSQRKQLFIFSNPPHGRRVKRIASKASDPDPLAGRLLNDPREDSVIKKLLIANRGEIAVRIVRACAEMGVRSVAVFSEADRHALHVKRADEAYFIGEDPLAGYLNPRKLVNLAVETGCDALHPGYGFLSENAELAEICAERGIKFVGPSADVIRRMGDKTEARRSMIKAGVPVTPGTEGNVKDLAEALREAERIGYPVMLKATSGGGGRGIRRCNSQAELESAYPRVISEATKAFGSAEVFLEKCIVEPKHIEAQVLADSFGNTVHLFERDCSIQRRNQKLIEIAPSPQLTPEQRAYIGDLAVRAAKAVGYENAGTVEFLLADGEVYFMEMNTRVQVEHTITEEITGIDIVREQIRIASGQPLSVKQEDIQHRGFSLQFRINAEDPRNNFLPCFGKITRYYAPGGPGVRTDTAIYTGYTIPPYYDSMCLKLVVWALTWEEALARGSRALDDMRVQGVKTTATYYQQILANPDFRSGQFNTSFVDNHPELLNYSIKRKPGELALAIAAAIAAHAGL

SEQ ID NO: 50 - protein, 603 aa, Pseudomonas stutzeri ATCC14405
MTAQKKITVTDTILRDAHQSLLATRMRTEDMLPICDKLDRVGYWSLEVWGGATFDACVRFLKEDPWERLRQLKAALPNTRLQMLLRGQNLLGYRHYSDDVVEAFCARAAENGIDVFRIFDAMNDVRNLETAIRAVKKSGKHAQGTIAYTTSPVHTVELFVEQARQMAAMGVDSIAIKDMAGLLTPFATGDLVRALKAEIDLPVFIHSHDTAGVASMCQLKAIENGADHIDTAISSMAWGTSHPGTESMVAALKGTPYDTGLDLELLQEIGLYFYAVRKKYHQFESEFTGVDTRVQVNQVPGGMISNLANQLKEQGALHRMDEVLAEIPKVRKDLGYPPLVTPTSQIVGTQAFFNVLAGERYKTITNEVKLYLQGRYGQAPAPVCERLRFMAIGSEEVIECRPADLLAPELDKLRKDIGGLAKSEEDVLTFAMFPDIGRKFLEEREAGTLQPEVLLPIPDGNVAAASVEGTPTEFVIDVHGESYRVDITGVGVKGEGKRHFYLSIDGMPEEVVFEPLNAFVGGGGSGRKQASAPGDVSTTMPGNVVDVLVAVGDVVKAGQTVLVSEAMKMETEIQAPIAGTVKAVHVAKGDRVNPGEVLIEIEG

SEQ ID NO: 51 - DNA, 3499 bp, Pseudomonas stutzeri ATCC14405
atgcgcatcaatgattttcgcatcgttttaccagtagttcgcctgcatttcgcggaacagtcaaacctgcggcgtttctgtctgactggtcaagaaacagtcattcctgacacacatataagtaaatacttatcccaaagaaaacaattattcattttcagtaatccccctcacgggcgtagggtgaaacgaatcgccagcaaggcgagtgatcctgacccgctcgcgggtcgcctgctcaacgatccgagggaagacagcgtgatcaagaagctgctgatcgccaaccgcggggaaatcgcggtgcgcatcgtccgcgcctgtgccgaaatgggcgtccgctcggtggcggtgttctccgaagccgaccgccatgcgctgcacgtcaagcgcgccgacgaggcctatttcatcggcgaggacccgctggccggctacctgaacccgcgcaagctggtaaacctggcggtagagaccggctgcgatgccctgcatcccggctatggattcctctccgagaacgccgaactggcggaaatctgcgccgagcgcgggatcaagttcgtcgggccttcggcagacgtgattcgccgcatgggcgacaagaccgaagcccgtcgcagcatgatcaaggccggcgtgccggtcacgccgggcaccgaaggcaacgtcaaggacctcgccgaggcgctgcgcgaagccgagcgcatcggttatccggtgatgctcaaggccacctccggtggtggcggtcgtggcattcgtcgctgcaactcgcaggcagagctcgagtcggcgtacccgcgggtgatctccgaagcgaccaaggccttcggcagtgccgaggtgttcctggaaaagtgcatcgtcgagcccaagcacatcgaggcgcaggtactggctgacagtttcggcaacaccgtgcacctgttcgagcgcgactgctcgatccagcggcgcaaccagaagctcatcgagatcgcccccagcccgcagctcacccccgagcagcgcgcctatatcggcgacctggccgtgcgtgccgccaaggcggtgggttacgagaacgccggtaccgtggagttcctgctcgccgatggcgaggtgtacttcatggagatgaacacccgggtgcaggtggagcacaccatcaccgaggaaatcaccggcatcgacatcgtgcgcgagcagatccgcatcgcttcgggccagccgctgtcggtcaagcaggaagacatccagcatcgcggcttctccctgcagttccgcatcaacgccgaggacccgcgcaacaacttcctgccctgcttcggcaagatcactcgctactacgctcccggcgggccgggcgtgcgcaccgacacggcgatctacaccggttacaccattccaccgtattacgactccatgtgcctgaagctggtggtctgggcgctgacctgggaagaggcgctggcccgcggctcgcgcgcgctggatgacatgcgcgtgcagggtgtgaagaccactgccacctactaccagcagattctcgccaatccggatttccgcagcggccagttcaataccagcttcgtcgacaaccatccggaactgctgaactactcgatcaaacgcaagccgggcgagctggccctggccattgccgccgccatcgccgcccacgcaggcctgtaaggaacgcaccatgactgcccagaagaaaatcaccgtcaccgacaccatcctgcgtgacgcccaccagtcgctgctggccacccgcatgcgcaccgaagacatgctgccgatctgcgacaagctcgaccgcgtcggctactggtcgctggaagtctggggtggcgccaccttcgacgcctgcgtgcgcttcctcaaggaggacccatgggagcgcctgcgccagctcaaggcagcgctgcccaatacccgcctgcagatgctgctgcgcgggcagaacctgctgggctaccgtcactacagcgatgacgtggtggaggcgttctgtgcccgtgcggcggagaacggcatcgacgtgttccgcatcttcgatgctatgaacgacgtacggaacctggaaaccgccatccgcgcggtgaagaagagcggcaagcacgcccagggcaccatcgcctataccaccagcccggtgcacaccgtcgagctgttcgtcgagcaggcgcggcagatggcggccatgggcgtcgactccatagccatcaaggacatggctggcctgctgaccccgttcgccactggcgatctggtccgcgcgctgaaggccgagatcgaccttccggtgttcatccattcccacgacaccgctggtgtggccagcatgtgccagctcaaggccatcgagaatggcgccgaccacatcgacaccgccatctccagcatggcctggggcaccagccatccgggcaccgagtccatggtcgccgcgctcaagggcacgccgtacgacaccggcctcgacctcgagctgctgcaggagatcggcctgtacttctacgccgtgcgcaagaagtatcaccagttcgaaagcgagttcaccggcgtcgacacccgcgtgcaggtcaaccaggtgcccggcgggatgatttccaacctcgccaaccagctcaaggagcagggtgcgctgcaccgcatggacgaagtgctggcggagattcccaaggtgcgcaaggacctcggctacccgccgctggtcacgccgacctcgcagatcgtcggcacccaggcgttcttcaatgtgctcgccggggagcgctacaagaccatcaccaacgaggtgaagctctacctgcagggccgctacggtcaggcgccggcaccggtctgcgagcgcctgcgcttcatggccatcggtagcgaggaggtcatcgagtgccgtccggccgacctgctggcaccggagctggacaagctgcgcaaggacatcggcgggctggccaagagcgaagaagacgtgctgaccttcgccatgttcccggacatcggccgcaagttcctcgaggagcgcgaggcaggcacgttgcagccggaagtgctgctgccgattcccgatggcaatgtcgcggcggccagcgtcgaaggtacgccgaccgagttcgtcatcgatgtccacggcgagagctaccgtgtcgacatcaccggtgtcggcgtcaagggcgagggcaagcggcacttctacctgtccatcgacggcatgccggaggaagtggtgttcgagccgttgaacgctttcgtcggcggtggcggcagcgggcgcaagcaggccagcgcgccgggcgacgtcagcaccaccatgccgggcaacgtggtcgacgtgctggtcgccgtcggcgacgtggtgaaggccgggcagacggtgctggtcagcgaggcgatgaagatggagaccgagatccaggcaccgatcgccggcaccgtgaaggccgttcacgtcgccaaaggtgaccgggtgaacccgggagaagtcttgatagagatcgagggctaa

SEQ ID NO: 52 - protein, 741 aa, Chlorobium limicola DSM 245
MASKSTIIYTKIDEAPALATYSLLPIIQAFTRGTGVDVETRDISLAGRIIANFPENLTEEQRIPDYLAQLGELALTPEANIIKLPNISASIPQLKAAIKELQEHGYNVPNYPEAPSNDEEKAIQARYAKVLGSAVNPVLREGNSDRRAPLSVKAYAQKHPHRMAAWSKDSKAHVSHMNEGDFYGSEQSVTVPAATTVRIEYVNGANEVTVLKEKTALLAGEVIDTSVMNVRKLRDFYAEQIEDAKSQGVLLSLHLKATMMKISDPIMFGHAVSVFYKDVFDKHGALLAELGVNVNNGLGDLYAKIQTLPEDKRAEIEADIMAVYKTRPELAMVDSDKGITNLHVPNDIIIDASMPVVVRDGGKMWGPDGQLHDCKAVIPDRCYATMYGEIVDDCRKNGAFDPSTIGSVPNVGLMAQKAEEYGSHDKTFTAAGDGVIRVVDADGTVLMSQKVETGDIFRMCQAKDAPIRDWVGLAVRRAKATGAPAVFWLDSNRAHDAQIIAKVNEYLKDLDTDGVEIKIMPPVEAMRFTLGRFRAGQDTISVTGNVLRDYLTDLFPIIELGTSAKMLSIVPLLNGGGLFETGAGGSAPKHVQQFQKEGYLRWDSLGEFSALAASLEHLAQTFGNPKAQVLADTLDQAIGKFLDNQKSPARKVGQIDNRGSHFYLALYWAEALAAQDSDAEMKARFAGVASSLAAKEELINAELIAAQGSPVDMGGYYQPDDEKTAAAMRPSGTLNAIIDAM

SEQ ID NO: 53 - DNA, 2226 bp, Chlorobium limicola DSM 245
atggcaagcaaatcgaccatcatctacaccaagatcgacgaggcgccggcactggcgacttactcgctgcttccgatcatccaggcctttacccgtggaaccggcgttgatgtcgagaccagggatatctcccttgccggcaggattatcgccaacttcccggagaatctgaccgaagagcagaggattcccgactacctcgcccagcttggcgagcttgcgctcaccccggaagccaacatcatcaaactgccgaatatcagcgcttcaattcctcagttgaaagccgcgatcaaagagcttcaggagcatggttacaatgttccgaactaccccgaagccccgtcgaatgacgaagagaaagcaattcaggcccgttatgccaaggtacttggcagtgccgtgaacccggtgcttcgcgaaggcaactccgaccgccgcgcgccgctttcggtcaaggcatacgcccagaaacatccgcaccgtatggctgcatggagcaaagactccaaggctcacgtttcccacatgaacgagggcgacttctacggcagcgagcagtccgtaaccgtgcctgccgccaccaccgttcgtatcgaatatgtcaacggcgccaacgaggtgaccgtgctgaaagagaaaaccgcactgctcgccggtgaagtgatcgacacgtcggtcatgaacgtgcgcaagctccgcgatttctacgctgagcagatcgaggatgccaaatcgcagggcgtgcttctttcgctgcacctgaaggctaccatgatgaagatctccgatccgatcatgttcggccacgctgtttcggtgttctacaaggatgtgtttgacaagcatggcgcattgctcgccgagcttggcgtgaacgtcaacaacggcctcggcgatctctacgctaaaatccagaccctgccggaagacaaacgcgccgagatcgaggctgacatcatggcggtctacaagacccgtcccgagctggcgatggtcgattccgacaagggcatcaccaacctgcacgtgccgaacgacatcatcatcgacgcttccatgccggtcgttgtgcgcgacggtggcaagatgtggggccccgacggtcagcttcacgactgcaaggccgtgattccggatcgctgctacgccaccatgtacggcgaaatcgtggacgactgccgcaagaacggcgcgttcgatccttccaccatcggcagcgtgccgaatgtcggcctgatggcgcagaaggctgaagagtatggttcgcacgacaagaccttcaccgcggctggcgacggcgtgattcgtgtggtcgatgccgacggtacggtactcatgtcgcagaaggtcgagaccggcgacattttccgcatgtgccaggccaaggatgctccgatccgcgactgggtcggccttgccgttcgccgcgccaaagccaccggtgctccggctgtgttctggctcgacagcaaccgtgctcacgatgcgcagatcatcgccaaggtgaacgagtatctcaaagacctcgacaccgacggcgtcgagatcaagatcatgcctccggtcgaagccatgcgcttcaccctcggccgtttccgtgccggacaggacaccatttcggtgaccggcaacgtgcttcgtgactacctcaccgacctgttcccgatcatcgagctcggcaccagcgccaagatgctttcgatcgttccgctgctcaacggtggtggcctgtttgaaaccggtgcaggtggttcggctcccaagcacgtgcagcagttccagaaagagggctacctccgctgggattcgctcggcgagttctcggctctggccgcgtcgcttgagcacctcgcacagaccttcggcaaccccaaggctcaggtgctggccgacacgctcgatcaggcgatcggtaagttcctcgacaaccagaagtcgcccgcccgcaaagtcggccagatcgacaaccgcggcagccacttctacctcgcgctctactgggcagaggctcttgccgcacaggattccgatgccgagatgaaggcacgtttcgctggcgttgcttcttcgctcgccgcgaaagaggagctcatcaacgccgagctgatcgccgcacagggcagcccggttgacatgggtggctactaccagcccgatgacgaaaagaccgccgcagccatgcgtccgagcggtacgctcaacgcgatcatcgacgccatgtga

SEQ ID NO: 54 - protein, 400 aa, Kosmotoga olearia TBF 19.5.1
MEGQKIKVENNSILVPNNPIIPYIAGDGIGPEIMRAAMLVWNSAISRVYAGKRKVVWKEIYAGEKAIEIFGDPLPEETIEAIKSHVVSIKSPLTTPVGRGYRSLNVKLRQVLDLYACIRPVKWIKGVPAPVKHPELLDVVIFRENTEDVYAGIEWKKGSQEAKKVIDFLRDTFNLEIRGDSGLGLKPISEFATKRITRKAIQYALENGRKSVTIVHKGNIMKYTEGAFVEWAYEVALNEFEGKVVSERELNEPVSEKLIVKDRIADNMFQQILLEPSEYDIMLLPNLNGDYLSDAVAAQVGGIGLVPGANIGDFVALFEPTHGTAPQLAGKEIANPTSLILSGAMMFDYIGWKEVGSIIRKAVEKTIMDGKMTIDLARKKGVEPLKTTEFAEEIIKNIEE

SEQ ID NO: 55 - DNA, 1203 bp, Kosmotoga olearia TBF 19.5.1
atggaaggacagaaaataaaggtagaaaacaacagtattttggttccaaataatcccataatcccatatatagcaggtgatggaatagggcccgaaataatgagggctgcgatgttggtgtggaattcagcaatttctcgtgtttatgcagggaaaagaaaagtcgtatggaaggaaatatatgcaggtgaaaaggctatagaaatctttggtgatccacttcctgaagaaacaatagaagctattaagagtcatgttgtttctataaaatcacctttgaccaccccggtcggaaggggatacaggagccttaatgtgaagctcaggcaggttctggatctgtatgcatgtataaggcctgtcaaatggataaaaggagttccagctccagttaagcacccggaacttttagatgtggtaattttccgtgagaacacggaagacgtgtacgctggaatagaatggaaaaaaggctcacaagaagcgaaaaaggttatcgactttttaagagatacgtttaatctggaaattagaggcgattcaggacttggattgaagcccataagtgaattcgctacgaagagaattacgagaaaagctattcaatacgccctggaaaatggcagaaagagtgtcaccatagtccataagggaaatataatgaaatacacagagggcgcttttgtagaatgggcttatgaagtggctttgaatgaatttgaaggcaaagtggtttcggagagagagttaaatgagcccgtatctgaaaaattgatcgtaaaagatagaatagcggataacatgttccagcagatactcttagaaccttcggagtacgatataatgctcctccctaacctgaatggagattatctgtctgatgctgttgcagctcaggttggtggtatagggttagttcctggtgcaaacataggagattttgtggctttgtttgaaccaacacacggtacagcaccgcaacttgctggaaaggaaatagcaaacccaacatccttgatattatccggtgctatgatgttcgattatattggatggaaagaagttggaagtattataagaaaagctgttgagaaaactataatggacgggaagatgaccatagatctcgcaagaaagaaaggtgtagagcctcttaaaaccacggaatttgcagaagaaatcattaaaaacattgaagaatag

SEQ ID NO: 56 - protein, 418 aa, Acinetobacter baumannii ACICU
MGYQKIVVPADGDKITVKADLSLNVPNHPIIPFIEGDGIGVDITPAMKKVVDAAILKAYGGKRSIEWMEVYCGEKANKIYGTYMPEETFEALREFVVSIKGPLTTPVGGGIRSLNVALRQELDLYVCVRPVRWFQGVPSPVQHPELTDMVIFRENSEDIYAGIEWKADSEEAKKVIKFLQEEMGVTKIRFPEGCGIGIKPVSKEGTQRLVRKAIQFAIDNDKPSVTLVHKGNIMKYTEGAFKEWGYELALDRFGGELIDGGPWVKIKNPKNGKDIIIKDVIADAFLQQILMRPADYSVIATLNLNGDYISDALAAEVGGIGIAPGANIGGAIAVYEATHGTAPKYAGQDKVNPGSIILSAEMMLRDMGWTEAADLIIKGISGAIAAKTVTYDFERLMPGATLLRCSEFGDAIIQHMED

SEQ ID NO: 57 - DNA, 1257 bp, Acinetobacter baumannii ACICU
atgggttatcagaagatcgtggttcctgccgacggtgataaaattacagtaaaagcagacctgtcactgaatgtaccaaatcatccaattattcctttcattgagggtgacggtattggtgtagatattacaccggcaatgaaaaaagttgttgatgcggcaattttaaaagcctatggcggcaaacgctctattgaatggatggaagtgtattgcggtgaaaaggccaataaaatttacggtacttatatgccggaagaaacctttgaagcgctgcgtgaatttgtagtttcaattaaaggccctttaactacaccagtcggtggtggcattcgttcacttaatgttgcactacgccaagaactggatttgtatgtatgtgtgcgtcctgtgcgttggttccaaggcgtcccttcacctgttcaacatcctgagttaactgacatggtgattttccgtgaaaactcggaagatatttatgcaggtattgaatggaaagcagattctgaagaagctaaaaaagttattaaattccttcaagaagaaatgggggtcacaaaaattcgtttccctgaaggatgtggtattggtattaaacccgtttccaaagaaggaacacagcgcttagttcgtaaggccattcagtttgcaatcgataatgacaaaccttcggtgactcttgttcataaaggcaacattatgaaatataccgaaggtgcctttaaagaatgggggtatgagttagcgctagatcgtttcggtggtgaattaatcgatggtggcccatgggttaaaattaagaatcctaaaaatggtaaagacatcattattaaagacgtgattgcagatgctttcttgcaacaaatcttgatgcgtcctgctgactactctgtaattgcaacccttaatttaaatggtgactatatttcagatgctttagcagcagaagtagggggaatcgggattgcgccaggtgcgaatattggtggagctattgcagtgtatgaagcaacgcatggcactgcacctaaatatgctgggcaagataaagtcaacccgggttcaattattctctctgctgaaatgatgctccgtgatatggggtggacagaagcagcggacctgattattaaaggtatttcaggagcgattgcagctaaaaccgtaacttacgattttgagcgtttaatgccgggagcgaccttgttacgttgctcagaatttggcgatgccataattcagcacatggaagattaa

SEQ ID NO: 58 - protein, 417 aa, Marine gamma proteobacterium HTCC2080
MSYKHIKVPESGDVITVNEDSSLSVPDKPIIPYIEGDGIGVDITPVMIDVVNAAVDKAYGGQKAISWMEIYTGEKAAELYEGDWFPEETLEAIKTYAVAIKGPLTTPVGGGFRSLNVALRQELDLYTCLRPVRWFEGVPSPVRRPEDCNMVIFRENSEDIYAGIEYQAGTPEAQKVVDFIINEMGATKIRFPTDVGIGIKPVSSAGTKRLVRKAIQYAIDQNLPSVTLVHKGNIMKFTEGAFRDWGYELAQEEFGGQLVDGGPWVEIKNPITGDPIIIKDVIADAMLQQVLTRPKEYSVVATLNLNGDYLSDALAAQVGGIGIAPGANLSDTVALFEATHGTAPKYAGQDKVNPGSLILSAEMMMRHLGWNEAADLIVDGVNGAIQAKTVTYDFERLMDGATLVSCSDFGKAIIKAM

SEQ ID NO: 59 - DNA, 1254 bp, Marine gamma proteobacterium HTCC2080
atgtcatacaagcacattaaggttccggaaagcggagacgtgatcacagtcaacgaggacagcagcctgtctgtgcctgacaagcctatcatcccttacatcgaaggtgacggaattggtgtcgacattacgccggtaatgattgatgtcgtcaatgccgcagtagacaaggcctacggggggcaaaaggccatatcttggatggagatatacaccggtgaaaaagcggctgaattgtacgaaggggactggtttcctgaggagacgctggaggccataaaaacctatgccgtcgctatcaagggaccattgacaaccccggtaggtggaggctttcgctcactcaacgtggcgctgcgtcaagagctagatctttacacctgcctgcggccggttcgctggtttgagggtgtcccttctcctgtacgtcgccctgaagactgcaacatggtgatctttcgagagaattcggaagatatatatgcgggcatcgaatatcaggctggaacacctgaagcgcaaaaggttgttgatttcatcattaatgaaatgggcgcgacaaagattcgttttccaacggacgtaggcattggcataaagcctgtctcctctgcgggaaccaagcgcttggttcgtaaagctattcagtatgccatcgatcaaaatctgccatctgtcacccttgtacacaaaggcaacatcatgaaatttaccgagggggcatttcgggattggggttacgagcttgctcaggaagagtttggcgggcagttagtagacggtggtccgtgggtggaaatcaaaaacccaataaccggtgatccgatcatcattaaagatgtgattgctgatgccatgctgcagcaggttttgacgcgtccaaaggaatacagtgtagtcgcaactttgaatcttaatggtgattatctttccgatgctttggccgctcaggtcggtggaattggtatcgctcctggcgctaacctttccgataccgttgcattgtttgaagccacccacggaacagcacctaaatacgctggtcaggacaaggttaatccgggctcgttgattttgtcggccgaaatgatgatgcgccacctaggatggaatgaggccgcagatcttatcgtcgatggtgtgaacggtgcgattcaagccaaaaccgtgacttatgactttgagcgattgatggacggggctactttggtctcatgttctgacttcggaaaagccataataaaagccatgtaa

SEQ ID NO: 60 - protein, 422 aa, Nitrosococcus halophilus Nc4
MAYDKISLPSDGEPITVKEDYSLEVPARPLIPFIEGDGIGVDITPVMRQVVDEAVAKAYGGERSLAWAEVYAGEKAAQVYGADQWLPAETLDVLRQFVVSIKGPLTTPVGKGIRSLNVAIRQTLDLYACIRPVRYFSGTPSPLADPSRTNMVVFRENTEDIYAGIEWAARSPEAKQVIEFLQQQMGVEKIRFPESSGIGIKPVSQEGSQRLIRKALQYAIDNDRRSVTLVHKGNIMKFTEGAFCDWGYALAQEEFGARPIDGGPWCEFTNPKSGGKIIVKDAIADNFLQQILLRPEEYDVIATLNLNGDYISDALAAQVGGIGMAPGANMGDRVAVFEATHGTAPKYAGQDRVNPSSIILSGEMMLRHLGWNEAADLIIQGISGAIAAKRVTYDLARLMEGATQVPCSGFGKAIIEHMDVSS

SEQ ID NO: 61 - DNA, 1269 bp, Nitrosococcus halophilus Nc4
atggcctatgacaagatttcccttccctccgatggcgaacccattaccgtcaaggaggactacagccttgaagtccccgcccgtcccctcattccctttatagaaggggatggcattggggtggatatcaccccggtgatgcgccaggtggtggatgaggcggtggcgaaggcctatgggggagagcgttccctggcctgggccgaggtgtatgcaggggagaaggccgcgcaagtgtatggcgccgatcaatggttgccggcggagactttggatgtcctgcggcaattcgtggtgtctatcaagggaccgctaaccacgccggtcggcaaaggtatccgttctcttaatgtggcgatccgccaaaccttggatctttatgcctgtatccggccggtccgttatttttcgggcacgccgagccctctggctgatccctcccgcaccaatatggtggtgtttcgggaaaataccgaggatatctatgccgggatcgagtgggcggcccgttcgccggaggcgaagcaggtcattgagtttttacaacagcagatgggggtggaaaaaatccgtttcccggaaagctccggcattggcattaaaccggtatcccaggaaggttctcaacgcctgatccgcaaagccctgcaatacgccatcgataatgatcgccgttcggtgaccctagtgcataaggggaacatcatgaagtttaccgaaggcgccttctgtgactggggttatgccttggcccaggaggagtttggcgcccggcccattgatggggggccctggtgtgaattcacgaatcctaaaagcggcggcaaaattattgtcaaagacgcgattgccgataattttctccaacagatcctgctccgccccgaggaatatgatgtcattgcgaccctgaatcttaatggagattacatttctgacgctttagcggcccaagtggggggaattggcatggcgccgggagcgaacatgggggatagggtcgccgtgtttgaggccacccacgggacggcccccaagtatgccggtcaggatcgggtcaatcccagcagcattatcctttcaggggaaatgatgttgcgtcatctcggctggaatgaagcggcggatctcatcatccaagggatttcgggggctatcgccgccaagagggtgacttacgatctagcccgattgatggaaggcgccacccaagtaccctgttctggatttggaaaggcgattatcgagcatatggacgtttccagctag

SEQ ID NO: 62 - protein, 432 aa, Corynebacterium glutamicum ATCC 13032
MSNVGKPRTAQEIQQDWDTNPRWNGITRDYTADQVADLQGSVIEEHTLARRGSEILWDAVTQEGDGYINALGALTGNQAVQQVRAGLKAVYLSGWQVAGDANLSGHTYPDQSLYPANSVPSVVRRINNALLRSDEIARTEGDTSVDNWVVPIVADGEAGFGGALNVYELQKAMIAAGAAGTHWEDQLASEKKCGHLGGKVLIPTQQHIRTLNSARLAADVANTPTVVIARTDAEAATLITSDVDERDQPFITGERTAEGYYHVKNGLEPCIARAKSYAPYADMIWMETGTPDLELAKKFAEGVRSEFPDQLLSYNCSPSFNWSAHLEADEIAKFQKELGAMGFKFQFITLAGFHSLNYGMFDLAYGYAREGMTSFVDLQNREFKAAEERGFTAVKHQREVGAGYFDQIATTVDPNSSTTALKGSTEEGQFHN

SEQ ID NO: 63 - DNA, 1299 bp, Corynebacterium glutamicum ATCC 13032
atgtcaaacgttggaaagccacgtaccgcacaggaaatccagcaggattgggacaccaaccctcgttggaacggcatcacccgcgactacaccgcagaccaggtagctgatctgcagggttccgtcatcgaggagcacactcttgctcgccgcggctcagagatcctctgggacgcagtcacccaggaaggtgacggatacatcaacgcgcttggcgcactcaccggtaaccaggctgttcagcaggttcgtgcaggcctgaaggctgtctacctgtccggttggcaggtcgcaggtgacgccaacctctccggccacacctaccctgaccagtccctctacccagcgaactccgttccaagcgtcgttcgtcgcatcaacaacgcactgctgcgttccgatgaaatcgcacgcaccgaaggcgacacctccgttgacaactgggttgtcccaatcgtcgcggacggcgaagctggcttcggtggagcactcaacgtctacgaactccagaaggcaatgatcgcagctggcgctgcaggcacccactgggaagaccagctcgcttctgaaaagaagtgtggccacctcggcggcaaggttctgatcccaacccagcagcacatccgcaccctgaactctgcccgccttgcagcagacgttgcaaacaccccaactgttgttatcgcacgtaccgacgctgaggcagcaaccctgatcacctctgacgttgatgagcgcgaccaaccattcatcaccggtgagcgcaccgcagaaggctactaccacgtcaagaatggtctcgagccatgtatcgcacgtgcaaagtcctacgcaccatacgcagatatgatctggatggagaccggcacccctgacctggagctcgctaagaagttcgctgaaggcgttcgctctgagttcccagaccagctgctgtcctacaactgctccccatccttcaactggtctgcacacctcgaggcagatgagatcgctaagttccagaaggaactcggcgcaatgggcttcaagttccagttcatcaccctcgcaggcttccactccctcaactacggcatgttcgacctggcttacggatacgctcgcgaaggcatgacctccttcgttgacctgcagaaccgtgagttcaaggcagctgaagagcgtggcttcaccgctgttaagcaccagcgtgaggttggcgcaggctacttcgaccagatcgcaaccaccgttgacccgaactcttctaccaccgctttgaagggttccactgaagaaggccagttccacaactag

SEQ ID NO: 64 - protein, 431 aa, Gordonia alkanivorans NBRC 16433
MSNVGKPRTAAEIQQDWDTNPRWKGIKRDYTAEQVAQLQGSVVEEHTLARRGAEILWDGVTKGDGSYINALGALTGNQAVQQVRAGLKAVYLSGWQVAGDANLSGHTYPDQSLYPANSVPNVVRRINNALLRADEIARVEGDDSVDNWVVPIVADGEAGFGGALNVYELQKAMIAAGAAGTHWEDQLASEKKCGHLGGKVLIPTQQHIRTLNSARLAADVAGVPTVVIARTDAEAATLITSDVDDRDKQFVTGERTAEGYYHVKNGIEPCIERAKSYAPYADMIWMETGTPDLELARKFAEAVKAEYPDQLLSYNCSPSFNWSKHLDDSTIAKFQNELGAMGFTFQFITLAGFHSLNYGMFDLAYGYAREQMTAFVDLQNREFKAADERGFTAVKHQREVGAGYFDSIATTVDPNTSTAALKGSTEEGQFH

SEQ ID NO: 65 - DNA, 1296 bp, Gordonia alkanivorans NBRC 16433
atgagcaacgtcggaaagccccgcaccgccgcggagatccagcaggactgggacaccaacccccgctggaagggcatcaagcgcgactacaccgccgagcaggtcgctcagctccagggttcggtcgtcgaggagcacaccctcgcccgccgtggcgccgagatcctgtgggacggcgtgaccaagggtgacggttcctacatcaacgctctcggcgccctcaccggcaaccaggccgtgcagcaggtccgcgccggcctgaaggccgtgtacctgtcgggttggcaggtcgccggtgacgccaacctgtccggccacacctaccccgaccagtcgctgtacccggcgaactcggttcccaacgttgttcgtcgcatcaacaacgcgctgctccgcgccgacgagatcgcccgcgtcgagggtgacgactcggtcgacaactgggtcgtgccgatcgtcgccgatggtgaggccggcttcggtggcgctctcaacgtctacgagctccagaaggccatgatcgccgcgggtgctgccggtacccactgggaggatcagctcgcctcggagaagaagtgcggccacctcggtggcaaggtgctcatcccgacccagcagcacatccgcaccctgaactcggcccgcctggccgccgacgtcgccggtgtccccaccgtcgtcatcgcgcgtaccgacgccgaggccgcgaccctcatcacctccgatgtggacgaccgcgacaagcagttcgtcaccggtgagcgcaccgccgagggctactaccacgtgaagaacggcatcgagccgtgcatcgagcgtgcgaagtcctacgctccgtacgccgacatgatctggatggagaccggtaccccggatctcgagctggctcgcaagttcgccgaggccgtcaaggccgagtaccccgaccagctgctgtcctacaactgcagcccgtcgttcaactggagcaagcacctcgacgacagcaccatcgccaagttccagaacgagctgggcgccatgggcttcaccttccagttcatcaccctggccggcttccactcgctcaactacggcatgttcgaccttgcctacggttacgcccgcgagcagatgaccgccttcgtcgacctgcagaaccgcgagttcaaggcagccgacgagcgtggcttcaccgccgtcaagcaccagcgtgaggtcggcgccgggtacttcgacagcatcgccaccaccgtcgacccgaacacctcgaccgcagctctcaagggctcgaccgaagagggccagttccactag

SEQ ID NO: 66 - protein, 429 aa, Nocardia farcinica IFM 10152
MSTTGTPKTAEEIQKDWDTNPRWKGVTRNYTAEQVVALQGNVVEEHTLARRGSEILWDLVNNEDYINSLGALTGNQAVQQVRAGLKAIYLSGWQVAGDANLSGHTYPDQSLYPANSVPAVVRRINNALLRADEIAKIEGDTSVENWLAPIVADGEAGFGGALNVYELQKAMIAAGVAGSHWEDQLASEKKCGHLGGKVLIPTQQHIRTLTSARLAADVAGVPTVVIARTDAEAATLITSDVDERDRPFITGERTSEGFYQVKNGIEPCIARAKAYAPYADLIWMETGTPDLELAKKFSEAVRSEFPDQLLAYNCSPSFNWSAHLDDSTIAKFQKELGAMGFKFQFITLAGFHSLNYGMFDLAYGYAREGMTAFVDLQNREFKAAEERGFTAIKHQREVGAGYFDAIATTVDPNTSTAALKGSTEEGQFH

SEQ ID NO: 67 - DNA, 1290 bp, Nocardia farcinica IFM 10152
atgtcgaccaccggcaccccgaagaccgctgaggagatccagaaggattgggacaccaaccctcgctggaagggcgtcacccgtaactacaccgccgagcaggtggttgcgcttcagggcaacgtcgtcgaggagcacaccctcgcccgtcgcggctcggagatcctgtgggacctcgtcaacaacgaggactacatcaactcgctgggcgccctcaccggcaaccaggccgtgcagcaggtccgcgccggcctgaaggccatctacctgtccggctggcaggtcgccggtgacgcgaacctctcgggtcacacctaccccgaccagtcgctgtacccggccaactcggttccggccgtggtccgccgcatcaacaacgcgctgctgcgcgccgacgagatcgccaagatcgagggcgacacctccgtcgagaactggctggccccgatcgtggccgacggtgaggcgggcttcggtggcgcgctcaacgtctacgagctgcagaaggccatgatcgccgccggtgtcgccggctcgcactgggaagaccagctggcctcggagaagaagtgcggccacctgggcggcaaggtgctcatccccacccagcagcacatccgcaccctgacctccgcgcgtctggccgccgacgtggccggtgtgccgaccgtcgtcatcgcccgcaccgatgccgaggccgccaccctgatcacctccgacgtggacgagcgcgaccgcccgttcatcaccggtgagcgcacctccgagggcttctaccaggtcaagaacggcatcgagccctgcatcgcccgcgccaaggcctacgcgccctacgcggacctgatctggatggagaccggcaccccggacctcgagctggccaagaagttctccgaggccgtgcgcagcgagttcccggaccagctgctggcctacaactgctcgccgtcgttcaactggtcggcgcacctggacgacagcaccatcgccaagttccagaaggagctgggcgcgatgggcttcaagttccagttcatcaccctggcgggcttccactcgctcaactacggcatgttcgacctggcctacggctacgcccgcgagggcatgaccgccttcgtcgacctgcagaaccgcgagttcaaggccgccgaggagcgtggcttcaccgccatcaagcaccagcgcgaggtcggcgcgggctacttcgacgccatcgccaccaccgtcgacccgaacacctcgacggccgcgctgaagggctccaccgaagagggtcagttccactga

SEQ ID NO: 68 - protein, 429 aa, Rhodococcus pyridinivorans AK37
MSTTGTPRTAEEIQKDWDTNPRWKGITRNYTAEQVAKLQGNVVEEATLARRGSEILWDLVNNEDYINSLGALTGNQAVQQVRAGLKAIYLSGWQVAGDANLSGHTYPDQSLYPANSVPQVVRRINNALLRADEIAKVEGDTSVDNWLAPIVADGEAGFGGALNVYELQKAMIAAGVAGSHWEDQLASEKKCGHLGGKVLIPTQQHIRTLTSARLAADVADVPTVVIARTDAEAATLITSDVDERDRPFITGERTAEGFYHVKNGIEPCIARAKAYAPYSDLIWMETGVPDLEVAKKFAEGVRSEFPDQLLAYNCSPSFNWKAHLDDATIAKFQKELGAMGFKFQFITLAGFHSLNYGMFDLAHGYAREGMTAFVDLQEREFKAAKERGFTAIKHQREVGAGYFDTIATTVDPNTSTAALKGSTEEGQFH

SEQ ID NO: 69 - DNA, 1290 bp, Rhodococcus pyridinivorans AK37
atgtcgaccaccggcaccccgaggactgcagaagagatccagaaggattgggacaccaatccgcgctggaaggggatcacccgcaactacaccgccgagcaggtcgccaagctgcagggcaacgtcgtcgaggaagccaccctcgctcgccgcggttccgagatcctgtgggacctcgtcaacaacgaggactacatcaactcgctcggcgccctcaccggtaaccaggcggtccagcaggtccgcgccggcctgaaggccatctacctctccggttggcaggtcgccggcgacgccaacctgtccggccacacctacccggaccagtcgctgtacccggcgaactcggttccgcaggtcgtccgccgtatcaacaacgcgctgctgcgcgccgacgagatcgccaaggtcgagggcgacacttccgtcgacaactggctcgctccgatcgtcgccgacggtgaggccggcttcggtggcgccctcaacgtctacgagctgcagaaggccatgatcgcggccggcgtcgccggttcccactgggaggaccagctcgcgtcggagaagaagtgcggtcacctcggtggcaaggtgctcatccccacgcagcagcacatccgcaccctgacctcggctcgcctcgcggccgacgtcgcggacgtcccgaccgtggtcatcgcccgcaccgacgccgaggccgcgaccctcatcacctccgatgtcgacgagcgtgaccgcccgttcatcaccggtgagcgcaccgccgagggcttctaccacgtcaagaacggcatcgagccctgcatcgcccgtgcgaaggcctacgctccgtactccgacctcatctggatggagaccggtgttccggacctcgaggtcgccaagaagttcgccgagggcgtccgcagcgagttcccggaccagctgctggcctacaactgctcgccgtccttcaactggaaggctcacctggacgacgcgaccatcgcgaagttccagaaggagctcggcgcgatgggcttcaagttccagttcatcaccctcgccggcttccactcgctcaactacggcatgttcgacctggcgcacggctacgcccgcgagggcatgacggccttcgtcgacctgcaggagcgcgagttcaaggcggccaaggagcgcggcttcaccgccatcaagcaccagcgtgaggtcggtgccggctacttcgacaccatcgccaccaccgtcgatcccaacacctccacggctgccctgaagggctccaccgaggaaggccagttccactag

SEQ ID NO: 70 - protein, 429 aa, Rhodococcus jostii RHA1
MSTTGTPKTAAEIQQDWDTNPRWKGVTRNYTAEQVTKLQGTVVEEQTLARRGSEILWDLVNNEDYINSLGALTGNQAVQQVRAGLKAIYLSGWQVAGDANLSGHTYPDQSLYPANSVPQVVRRINNALLRADEIAKVEGDTSVDNWLAPIVADGEAGFGGALNVYELQKAMIAAGVAGSHWEDQLASEKKCGHLGGKVLVPTQQHIRTLTSARLAADVADVPTVVIARTDAEAATLITSDVDERDQQFLDGTRTAEGFFGIKNGIEPCIARAKAYAPYADLIWMETGVPDLEVAKKFAESVRSEFPDQLLAYNCSPSFNWKAHLDDATIAKFQKELGAMGFKFQFITLAGFHSLNYGMFDLAHGYAREGMTAFVDLQEREFKAAEERGFTAIKHQREVGAGYFDSIATTVDPNTSTAALKGSTEEGQFH

SEQ ID NO: 71 - DNA, 1290 bp, Rhodococcus jostii RHA1
atgtcgaccaccggcaccccgaagaccgcagctgaaatccagcaggattgggacaccaacccgcgctggaagggagtaacccgcaactacacggcggagcaggtcaccaagctccagggcaccgttgtcgaagagcagaccctcgcacgccgtggttccgagatcctctgggacctcgtgaacaacgaggactacatcaactcgctgggcgcgctgaccggcaaccaggccgttcagcaggtccgtgcaggcctcaaggccatctacctgtccggttggcaggtcgccggtgacgcgaacctgtccggacatacctaccccgaccagagcctctacccggccaactcggtcccgcaggtcgtgcgccgcatcaacaatgcgctgctgcgtgccgacgagatcgccaaggtcgagggcgacacctccgtcgacaactggctcgccccgatcgtcgccgacggagaagcaggcttcggtggcgcgctcaacgtctacgagctgcagaaggccatgatcgcggccggcgtcgccggttcgcactgggaagaccagctcgcgtcggagaagaagtgtggccacctcggtggcaaggtcctcgtccccacgcagcagcacatccgcaccctgacctcggctcgcctcgccgccgacgtcgcggacgttcccaccgtggtcatcgcccgcaccgatgccgaggccgcgaccctcatcacgtccgacgtcgacgagcgcgaccagcagttcctggacggaacccgcaccgccgagggcttcttcggtatcaagaacggcatcgagccctgcatcgcgcgcgccaaggcctacgccccgtacgccgacctcatctggatggagaccggcgtgccggacctcgaggtcgccaagaagttcgccgagtcggttcgcagcgagttcccggaccagctgctcgcgtacaactgctcgccgtccttcaactggaaggcgcacctggacgacgccaccatcgcgaagttccagaaggagctcggcgcgatgggcttcaagttccagttcatcaccctggccggcttccactcgctcaactacggcatgttcgacctggcgcacggctacgcccgcgagggcatgaccgccttcgtcgacctgcaggagcgcgagttcaaggccgccgaggagcgtggcttcaccgccatcaagcatcagcgtgaggtcggtgccggctacttcgacagcatcgccaccacggtcgaccccaacacctcgacggctgccctgaagggctccaccgaagagggtcagttccactga

SEQ ID NO: 72 - DNA, 375 bp, Pyrococcus furiosis
atgattagtgaaaccataagaagtggggactggaaaggagaaaagcacgtccccgttatagagtatgaaagagaaggggagcttgttaaagttaaggtgcaggttggtaaagaaatcccgcatccaaacaccactgagcaccacatcagatacatagagctttatttcttaccagaaggtgagaactttgtttaccaggttggaagagttgagtttacagctcacggagagtctgtaaacggcccaaacacgagtgatgtgtacacagaacccatagcttactttgtgctcaagactaagaagaagggcaagctctatgctcttagctactgtaacatccacggcctttgggaaaacgaagtcactttagagtga

SEQ ID NO: 73 - protein, 124 aa, Pyrococcus furiosis
MISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSYCNIHGLWENEVTLE

SEQ ID NO: 74 - DNA, 375 bp, Pyrococcus furiosis
atgattagtgaaaccataagaagtggggactggaaaggagaaaagcacgtccccgttatagagtatgaaagagaaggggagcttgttaaagttaaggtgcaggttggtaaagaaatcccgcatccaaacaccactgagcaccacatcagatacatagagctttatttcttaccagaaggtgagaactttgtttaccaggttggaagagttgagtttacagctcacggagagtctgtaaacggcccaaacacgagtgatgtgtacacagaacccatagcttactttgtgctcaagactaagaagaagggcaagctctatgctcttagcgactgtaacatccacggcctttgggaaaacgaagtcactttagagtga

SEQ ID NO: 75 - protein, 124 aa, Pyrococcus furiosis
MISETIRSGDWKGEKHVPVIEYEREGELVKVKVQVGKEIPHPNTTEHHIRYIELYFLPEGENFVYQVGRVEFTAHGESVNGPNTSDVYTEPIAYFVLKTKKKGKLYALSDCNIHGLWENEVTLE

SEQ ID NO: 76 - DNA, 861 bp, Camelina sativa
atggcagaaaacaaagaagaagatgttaagcttggagctaacaaattcagagaaacacagccattaggaacagctgctcaaacagacaaagattacaaagaaccaccaccagctcctttgtttgaaccaggggaattatcatcatggtcattttacagagctggaattgcagaatttatggctactttcttgtttttgtacatcactatcttgactgttatgggtcttaagagatctgatagtctgtgtagttcagttggtattcaaggtgttgcttgggcttttggtggtatgatctttgctttggtttactgtactgctggtatctcaggaggacacatcaacccagctgtgacctttggattgttcttggcaaggaaactgtccttaaccagggctattttctacatagtgatgcaatgccttggtgcaatttgtggtgctggtgttgtgaagggattcatggttggtccataccagagacttggtggtggtgctaatgttgttaaccatggttacaccaaaggtgatggccttggtgctgaaattattggcacttttgtccttgtttacactgttttctctgctactgatgctaagagaaatgccagagactcacatgttcctattttggcaccacttcccatcggattcgcggttttcttggttcatttggccaccattcccatcaccggaactggcatcaaccccgctaggagtcttggagctgcgatcatctacaacacagaccaggcatgggacgaccactggatcttttgggttggaccattcattggagctgcacttgctgcagtttaccatcaaataatcatcagagccattccattccacaagtcgtct

SEQ ID NO: 77 - protein, 287 aa, Camelina sativa
MAENKEEDVKLGANKFRETQPLGTAAQTDKDYKEPPPAPLFEPGELSSWSFYRAGIAEFMATFLFLYITILTVMGLKRSDSLCSSVGIQGVAWAFGGMIFALVYCTAGISGGHINPAVTFGLFLARKLSLTRAIFYIVMQCLGAICGAGVVKGFMVGPYQRLGGGANVVNHGYTKGDGLGAEIIGTFVLVYTVFSATDAKRNARDSHVPILAPLPIGFAVFLVHLATIPITGTGINPARSLGAAIIYNTDQAWDDHWIFWVGPFIGAALAAVYHQIIIRAIPFHKSS

SEQ ID NO: 78 - DNA, 2496 bp, Synechococcus sp. PCC 7002
ccgtaagcatcaacgattctttacatcatcatccatcggcgcgacttgctcacatcgcagcattaagattgcagttgccatagccacaatcccagaaaaaattcacgatccagtacccgaaagcctttttttaaaccaattttagataagttttagttatttttttatccaaaaagacttaagtccagcttatttacatgtcatggccttaggactatattaaatctcacatccatagtcgaaagactatcaacaggccaagtttaagggcaatgtccttgaggattctgccctttctctcagtttttcatcattgattcttcgatcaattgagtacagcacctagttaaagcaaacacaaatatatgaatcaatacagtcatcgtaaatttttgatcaccactggcgtggcagcgggcagcttatccatattttctttgtagtaattagagttttagcacagaaacaattggaactttcttgggcattttaaacaattttatatttatcgaggaggaatctactgttatgagacaacagcaacttttttggctgactactttgatcgttgggggcaatatttttcaggctgctacgccactacaggcccaggaaattaatttgacaacatcgctgagttcaccaacactacaggattctcgctatctagcctcggcctccatgggacaaatggcctcagtatctagattacgggacgtgaagccgacggattgggcttatgaagcactacaaagtctggtggaacggtatggttgcattgttggttatccagatcaaacattccgcggcgatcgccccctgagccgttatgaatttgccgccggactaaatgcttgcctcaatgccctagaacggcagatccaaggcaataatgccgatgtatcctccagcgatcttgcaaccctccggcgattgaccaacgagtttcaggcggaattagccaccctcggcacaagggttgatgatctcgaagcccgcaccagtgaactcgaaaaccaacaattttcaacgaccacaaaactgaatggagaagctattttctctatcagtggggcaacgggtggtgaaccagagggcaacgatgctcagattaccttcaataatcgtctgcggctgaatttgaccaccagttttaccggaaaagatgccctgattactggcttacaagcctacaatttttcggcgggtaaatctattacaggtacaggtaacgttgccgaaactctctttcccaatgatgcctctatccttggggatagcatgactaacctcgcctgggaaccacaatttgctggtttgaatccacaaaatctacaacctagttgcggtaacaatagcctttgtctgtacaagttgctctatgttagaccgatcacagataaattaacggcatttattggcccgaaggcggaagttaccgatgcctttccggcgattcttccctttgctagtgaaggccagggagcactttctcgctttgcaactttgaatccagtattgcggatgtctgggggaaccagtggtacaggactcgcttccgcagctggctttatctataaacccaatgatgtcatcgattggcgggcactctatgggtcagtgaatgcggcaatccctggtaatgaaggttttccggggacgccgttgggggctggcttgttcaatggcagttttatcgccgcaacacaattgacgcttcatcctaatgacaagcttgatctaggtctgaactatgcctacagctaccaccagatcaatattgcgggtacgggtttaacaggagctgagacgcgtattcttggcgatctaccactgaccaccccagtacgatttaactcctttggggcaacagtaaactggcgcgtcagtccaaaagttaacctgacaggttatggggcatacatcatgacagatcaagcgaatagtggctctgcctatacaaatctaagcagttggatggcgggtctgtattttccagatgcattcgcgaagggcaatgcggcagggattttgtttggtcaaccactttatcgggtagatgcgggtaatggggcgagtttaagtccagcaaacattggcgatcgccaaaccccctaccaactggaagccttttatcgccatcaaatcaatgatcacatcagcattacgccgggggcatttgtgattttcaatccagaaggagatgcccaaaatgaaacaaccagcgtttttgcgttgcgtacgacttataccttctagaactaactgatcaccattttacttagtagaaacttatgagtgtttttgttgcggctgatagtattgataaagtatttccgttgtcgggggtggtgaatatattaccctttaatattttttaccttcataaatcatgttcaaaactttaatcaaaaatagtgcggcgatcgcgtttgtacttttaggttccatagccgttattcctggggcaagttcccaaattagtgctactccctt

SEQ ID NO: 79 - protein, 576 aa, Synechococcus sp. PCC 7002
MRQQQLFWLTTLIVGGNIFQAATPLQAQEINLTTSLSSPTLQDSRYLASASMGQMASVSRLRDVKPTDWAYEALQSLVERYGCIVGYPDQTFRGDRPLSRYEFAAGLNACLNALERQIQGNNADVSSSDLATLRRLTNEFQAELATLGTRVDDLEARTSELENQQFSTTTKLNGEAIFSISGATGGEPEGNDAQITFNNRLRLNLTTSFTGKDALITGLQAYNFSAGKSITGTGNVAETLFPNDASILGDSMTNLAWEPQFAGLNPQNLQPSCGNNSLCLYKLLYVRPITDKLTAFIGPKAEVTDAFPAILPFASEGQGALSRFATLNPVLRMSGGTSGTGLASAAGFIYKPNDVIDWRALYGSVNAAIPGNEGFPGTPLGAGLFNGSFIAATQLTLHPNDKLDLGLNYAYSYHQINIAGTGLTGAETRILGDLPLTTPVRFNSFGATVNWRVSPKVNLTGYGAYIMTDQANSGSAYTNLSSWMAGLYFPDAFAKGNAAGILFGQPLYRVDAGNGASLSPANIGDRQTPYQLEAFYRHQINDHISITPGAFVIFNPEGDAQNETTSVFALRTTYTF

SEQ ID NO: 80 - DNA, 948 bp, Thioalkalivibrio sp. K90mix
atggcttttgatccggtagttctgttcttcctgctcggggcgattgccgggctggccaagtcggacctcaagatcccgatggcgatctacgaggcactgtcgatttacctcctgctggccatcggcttgcatggtggcgtgaagctggcggaaagcgagctggtgccgctcatcctgcctggccttgcggtgctgatggtcggggccctgatcccgctgctggcgttcccggtgctgcgctggctggggcatatgccgcgcgcggattcggcctccatcgccgcgcactacgggtcggtcagtgtggtgacgttctcggtggcggtggcctttctcgcggcccgagggatcgactacgagggccacatggtggtcttcctggtgctgctggagatgccggcactggtgatcggcatcctgctggcgcgcatgggcacgaagggaccggtgcaatggggcaagaccatgcacgaggtctttttcggcaagagcatcttcctgctcgccggtgggctggtgatcggattcgtggccggtcccgaactgatggacccactggagccgatgttcttcgatctgttcaagggcgtgctggccctgttcctgctggagatggggctggtcgcctcgagccggatcgccgaggtgcgccagtacgggctgttcctggtagtgttcgcgatcgtgatgccggtggtctcggcgatcctcgggatcctgctgggctggggcctgggcatgagcctgggcggtacgctgctgctggctaccctgtacgcgagtgcgtcctacatcgccgcacccgcggccatgcggatcgcggtccccaaggccaaccccgcgctgtcgatcggggcctcgctgggggttaccttcccgttcaatattttcctgggcgtcccgctgtatttctggatgacccagtggctctactcgttgggaggctag

SEQ ID NO: 81 - protein, 315 aa, Thioalkalivibrio sp. K90mix
MAFDPVVLFFLLGAIAGLAKSDLKIPMAIYEALSIYLLLAIGLHGGVKLAESELVPLILPGLAVLMVGALIPLLAFPVLRWLGHMPRADSASIAAHYGSVSVVTFSVAVAFLAARGIDYEGHMVVFLVLLEMPALVIGILLARMGTKGPVQWGKTMHEVFFGKSIFLLAGGLVIGFVAGPELMDPLEPMFFDLFKGVLALFLLEMGLVASSRIAEVRQYGLFLVVFAIVMPVVSAILGILLGWGLGMSLGGTLLLATLYASASYIAAPAAMRIAVPKANPALSIGASLGVTFPFNIFLGVPLYFWMTQWLYSLGG

SEQ ID NO: 82 - protein, 57 aa, Nicotiana tabacum
MASSVLSSAAVATRSNVAQANMVAPFTGLKSAASFPVSRKQNLDITSIASNGGRVQC

SEQ ID NO: 83 - protein, 25 aa, Saccharomyces cerevisiae
MLSLRQSIRFFKPATRTLCSSRYLL

SEQ ID NO: 84 - protein, 78 aa, Arabidopsis thaliana
MYLTASSSASSSIIRAASSRSSSLFSFRSVLSPSVSSTSPSSLLARRSFGTISPAFRRWSHSFHSKPSPFRFTSQIRA

SEQ ID NO: 85 - protein, 19 aa, Saccharomyces cerevisiae
MLSARSAIKRPIVRGLATV

SEQ ID NO: 86 - protein, 26 aa, Arabidopsis thaliana
MRILPKSGGGALCLLFVFALCSVAHS

SEQ ID NO: 87 - protein, 9 aa, Artificial
  PTS-2 signal sequence
  misc_feature (3)..(7): Xaa can be any naturally occurring amino acid
RLXXXXXHL

SEQ ID NO: 88 - protein, 10 aa, Artificial
  PTS-2 signal sequence
MRLSIHAEHL

SEQ ID NO: 89 - protein, 85 aa, Arabidopsis thaliana
MLRTVSCLASRSSSSLFFRFFRQFPRSYMSLTSSTAALRVPSRNLRRISSPSVAGRRLLLRRGLRIPSAAVRSVNGQFSRLSVRA

SEQ ID NO: 90 - protein, 35 aa, Chlamydomonas reinhardtii
MALVARPVLSARVAASRPRVAARKAVRVSAKYGEN

SEQ ID NO: 91 - protein, 29 aa, Chlamydomonas reinhardtii
MQALSSRVNIAAKPQRAQRLVVRAEEVKA

SEQ ID NO: 92 - protein, 35 aa, Chlamydomonas reinhardtii
MQTLASRPSLRASARVAPRRAPRVAVVTKAALDPQ

SEQ ID NO: 93 - protein, 31 aa, Chlamydomonas reinhardtii
MQALATRPSAIRPTKAARRSSVVVRADGFIG

SEQ ID NO: 94 - protein, 51 aa, Chlamydomonas reinhardtii
MAFALASRKALQVTCKATGKKTAAKAAAPKSSGVEFYGPNRAKWLGPYSEN

SEQ ID NO: 95 - protein, 50 aa, Chlamydomonas reinhardtii
MAAVIAKSSVSAAVARPARSSVRPMAALKPAVKAAPVAAPAQANQMMVWT

SEQ ID NO: 96 - protein, 40 aa, Chlamydomonas reinhardtii
MAAMLASKQGAFMGRSSFAPAPKGVASRGSLQVVAGLKEV

SEQ ID NO: 97 - protein, 4 aa, Arabidopsis thaliana
CVVQ

SEQ ID NO: 98 - DNA, 516 bp, Pyrococcus furiosis
atggtcgtgaaaagaacaatgactaaaaagttcttggaagaagcctttgcaggcgaaagcatggcccatatgaggtatttgatctttgccgagaaagctgaacaagaaggatttccaaacatagccaagctgttcagggcaatagcttacgcagagtttgttcacgctaaaaaccacttcatagctctaggaaaattaggcaaaactccagaaaacttacagatgggaatagagggagaaacgttcgaagttgaggaaatgtacccagtatacaacaaagccgcagaattccaaggagaaaaggaagcagttagaacaacccactatgctttagaggcggagaagatccacgctgaactctatagaaaggcaaaagagaaagctgagaaaggggaagacattgaaataaagaaagtttacatatgcccaatctgtggatacaccgctgttgatgaggctccagaatactgtccagtttgtggagctccaaaagaaaagttcgttgtctttgaatga

SEQ ID NO: 99 - protein, 171 aa, Pyrococcus furiosis
MVVKRTMTKKFLEEAFAGESMAHMRYLIFAEKAEQEGFPNIAKLFRAIAYAEFVHAKNHFIALGKLGKTPENLQMGIEGETFEVEEMYPVYNKAAEFQGEKEAVRTTHYALEAEKIHAELYRKAKEKAEKGEDIEIKKVYICPICGYTAVDEAPEYCPVCGAPKEKFVVFE

SEQ ID NO: 100 - DNA, 1782 bp, Unknown
  Glyoxylate carboligase nucleotide sequence
atggctaagatgagggctgtggatgctgctatgtatgtgcttgaaaaggagggaataactaccgcatttggtgtgcctggtgctgctattaatcctttctattcagctatgagaaagcatggaggtatcagacacatattggcaaggcatgtggaaggtgctagtcatatggcagagggatacaccagagctactgctggaaacattggagtttgtcttggtactagtggaccagctggtacagatatgatcaccgcactctatagtgcttctgctgattctattcctatcttatgcatcacaggtcaagctccaagagcaaggcttcacaaagaagatttccaggctgtggatattgaggctatcgcaaagcctgtttctaaaatggctgtgactgttagagaagctgcacttgtgccaagggttttgcaacaggcttttcatttgatgagatcaggaaggcctggtccagtgctcgttgatcttcctttcgatgtgcaagttgctgaaattgagtttgatcctgatatgtatgaacctcttccagtgtacaagccagctgcatctagaatgcaaatcgaaaaagctgttgagatgttgattcaggcagagaggcctgtgatcgttgctggaggtggagttattaatgcagatgctgctgctcttttgcaacagtttgctgaactcacctcagtgcctgttatcccaactttaatgggttggggatgtattcctgatgatcacgagctcatggctggaatggtgggtttacaaactgcacatagatacggtaacgctacactcttagcatctgatatggttttcggtattggaaatagatttgctaacaggcacacaggttcagtggaaaagtacactgagggaagaaaaattgttcatattgatattgagcctacccagatcggtagggtgctttgcccagatttgggaatagtttctgatgctaaggcagctttaacacttttggtggaagttgctcaagagatgcagaaggcaggaagactcccatgtaggaaagaatgggttgctgagtgccaacagagaaagaggactctcctcagaaaaacacatttcgataacgtgcctgttaagccacaaagagtttatgaagagatgaacaaagcttttggtagggatgtgtgttacgttactacaatcggactttctcaaatagcagctgcacagatgttgcacgttttcaaagatagacattggataaactgtggacaggctggtcctcttggatggactatcccagctgcattgggtgtttgcgctgctgatcctaagagaaacgttgtggctataagtggagatttcgatttccaattcctcatcgaagagttagctgttggagcacagtttaaaataccatacattcacgtgttggttaataacgcttaccttggattgattagacaatcacagagggctttcgatatggattactgtgttcaacttgcattcgaaaatatcaactcttcagaagtgaatggttacggagttgatcatgtgaaggttgctgaaggtctcggatgcaaggcaataagagttttcaaacctgaagatattgctccagcatttgagcaagctaaagcacttatggctcagtacagagttcctgttgtggttgaagtgattttggagagggttacaaatatctcaatgggaagtgagctcgataacgttatggaattcgaggatattgctgataacgctgctgatgctccaactgagacttgttttatgcactacgaatga

SEQ ID NO: 101 - protein, 593 aa, Unknown
  Glyoxylate carboligase amino acid sequence
MAKMRAVDAAMYVLEKEGITTAFGVPGAAINPFYSAMRKHGGIRHILARHVEGASHMAEGYTRATAGNIGVCLGTSGPAGTDMITALYSASADSIPILCITGQAPRARLHKEDFQAVDIEAIAKPVSKMAVTVREAALVPRVLQQAFHLMRSGRPGPVLVDLPFDVQVAEIEFDPDMYEPLPVYKPAASRMQIEKAVEMLIQAERPVIVAGGGVINADAAALLQQFAELTSVPVIPTLMGWGCIPDDHELMAGMVGLQTAHRYGNATLLASDMVFGIGNRFANRHTGSVEKYTEGRKIVHIDIEPTQIGRVLCPDLGIVSDAKAALTLLVEVAQEMQKAGRLPCRKEWVAECQQRKRTLLRKTHFDNVPVKPQRVYEEMNKAFGRDVCYVTTIGLSQIAAAQMLHVFKDRHWINCGQAGPLGWTIPAALGVCAADPKRNVVAISGDFDFQFLIEELAVGAQFKIPYIHVLVNNAYLGLIRQSQRAFDMDYCVQLAFENINSSEVNGYGVDHVKVAEGLGCKAIRVFKPEDIAPAFEQAKALMAQYRVPVVVEVILERVTNISMGSELDNVMEFEDIADNAADAPTETCFMHYE

SEQ ID NO: 102 - DNA, 879 bp, Artificial
  Tartronic semialdehyde reductase nucleotide sequence
atgaagttaggttttatcggtctcggtattatgggaacaccaatggcaatcaatctcgcaagggctggacaccaattacacgttacagctattggacctgttgcagatgaacttttgtcacttggtgctgttagtgtggaaaccgcaagacaagttactgaggcttctgatataatctttattatggtgcctgatactccacaggttgaagaggtgctcttcggagagaatggttgtacaaaggcttcattaaagggaaaaaccatcgttgatatgtcttcaatcagtcctatagaaaccaaaagatttgctagacaagttaacgagcttggaggagattatttggatgcaccagtgagtggaggtgaaattggagctagagagggtactctttctatcatggttggaggagatgaagctgtttttgagagggtgaagcctctcttcgaactcctcggaaaaaatatcactctcgtgggtggtaacggagatggtcaaacatgcaaggttgcaaatcagataattgtggctttgaacatagaagcagtttctgaggctcttttgtttgcatcaaaagctggtgcagatccagttagagtgaggcaggcacttatgggaggtttcgctagttctagaatattggaagttcatggagagagaatgataaagagaacttttaatcctggattcaagatcgcactccaccaaaaagatctcaacttagctcttcagtctgctaaagcattggctctcaatcttccaaacactgctacatgtcaagagttgttcaatacctgcgctgcaaacggaggttcacagttggatcacagtgctctcgtgcaggctttagaactcatggcaaaccacaaactcgcataa

SEQ ID NO: 103 - protein, 292 aa, Unknown
  Tartronic semialdehyde amino acid sequence
MKLGFIGLGIMGTPMAINLARAGHQLHVTAIGPVADELLSLGAVSVETARQVTEASDIIFIMVPDTPQVEEVLFGENGCTKASLKGKTIVDMSSISPIETKRFARQVNELGGDYLDAPVSGGEIGAREGTLSIMVGGDEAVFERVKPLFELLGKNITLVGGNGDGQTCKVANQIIVALNIEAVSEALLFASKAGADPVRVRQALMGGFASSRILEVHGERMIKRTFNPGFKIALHQKDLNLALQSAKALALNLPNTATCQELFNTCAANGGSQLDHSALVQALELMANHKLA

SEQ ID NO: 104 - DNA, 608 bp, Camelina sativa
gttaaaaattctttaaatgaactttaataaatagtatatatttaattaaaaagcaatattgaaattttgaaaaccaaaaaaatgtatagtaattttgaaattcaaatcattgcaggaaattaaatacatatatggttttaggcataaatacactttccatatcatgatcacttgactaatattaatttggcatatttataatttcatagtaagatcttatttcagtctggtcataatattagacattatataatgtatatataatttatattagtgtttttgccaaatttgttcttggatactatagaaactaaaaagattaataacccaaactaaagaaatctaaaaacattcaaattaaattttgattggacaatatcaatttggtggtatatactaaaataaaagtatattacctgaaaatatcagaaatgatatatagcttttttatccttattaagagattttggtaaaggcacaccaccaattcaattatatatatactggagacgggcactacacagacaagacacacacacttataaataaacaaaaagcgaaacctccatctttttacatataaagatcatcatccaacaagaagaagg

SEQ ID NO: 105 - DNA, 541 bp, Camelina sativa
aatgaactaatgtgtatatatatgtatgacttactttcgaataatgaactaatgtgtatgtatgacttactttcgaatgaagaaagttagaaagaatacaaattgattcttatttcagttgttcacatgtaaacacgttatatggcatcttgacaaaaagaaatatcacttaattcacattgagaattcttttgttttcatataggactattatatatagcaacaatatgtatcctgtaaatttgaatcccaattgtaacagccatatataatattagcataactattggactaaatgtcatggttaacgtagttaatgtgctattgtaattaattgtcataccacgtaaaaatcaataaaaggtactaaaatcatttcatattttgcaactacaaatgataaacaaaagtagtatttatttttatatatattttaaaatacgtaatatcaagaaactgcttaaaatataagacaagaatcctctttcttccatctctatctctctccgtagacagtttgctcaagcccctcttcttgaag

SEQ ID NO: 106 - DNA, 1399 bp, Artificial
  cwii1 RNAi sequence
tgcaccagacttcaaactttgtgtctctctactcaactccgacccacgtggctcctctgccgacatctctggcctcgctctcatcctcatcgataaaatcaaggtgctggcgacaaagaccttaaccgagatcaacggtctatataaaaagagaccggaactaaaacaggctttggaccaatgtagtcgaagatacaaaacgattttaaatgctgatgttcccgaagccatcgaagctatctctaaaggagtccctaaattcggcgaagacggcgtgattgacgccggggtagaagcttctgtttgtgcccgggaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgagcacaaacagaagcttctaccccggcgtcaatcacgccgtcttcgccgaatttagggactcctttagagatagcttcgatggcttcgggaacatcagcatttaaaatcgttttgtatcttcgactacattggtccaaagcctgttttagttccggtctctttttatatagaccgttgatctcggttaaggtctttgtcgccagcaccttgattttatcgatgaggatgagagcgaggccagagatgtcggcagaggagccacgtgggtcggagttgagtagagagacacaaagtttgaagtctggtgcatt

SEQ ID NO: 107 - DNA, 1398 bp, Artificial
  cwii2 RNAi sequence
gtaccatgccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacttccaccgcaacttacatcgccggaaacctaacatccgctgcaaacgacgtcgtccttaaaaaggtgttacaagattgctccgagaagtatgctctcgccgctgattctctccgtcaaacaattcaagatcttgatgatgaagcttatgactatgccccgggaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgaggcatagtcataagcttcatcatcaagatcttgaattgtttgacggagagaatcagcggcgagagcatacttctcggagcaatcttgtaacacctttttaaggacgacgtcgtttgcagcggatgttaggtttccggcgatgtaagttgcggtggaagtggcgtttgtcataccaacgccgatcataatggctgcgagaccttttgtgtcggctgtgggacttcttgggtcggatttgagagccgagacacagaatttgtagttgttcgtggttttgcaagttgattcgattattgtcgcgttggc

SEQ ID NO: 108 - DNA, 2022 bp, Artificial
  cwii1-cwii2 RNAi sequence
tgcaccagacttcaaactttgtgtctctctactcaactccgacccacgtggctcctctgccgacatctctggcctcgctctcatcctcatcgataaaatcaaggtgctggcgacaaagaccttaaccgagatcaacggtctatataaaaagagaccggaactaaaacaggctttggaccaatgtagtcgaagatacaaaacgattttaaatgctgatgttcccgaagccatcgaagctatctctaaaggagtccctaaattcggcgaagacggcgtgattgacgccggggtagaagcttctgtttgtgtctagaccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacttccaccgcaacttacatcgccggaaacctaacatccgctgcaaacgacgtcgtccttaaaaaggtgttacaagattgctccgagaagtatgctctcgccgctgattctctccgtcaaacaattcaagatcttgatgatgaagcttatgactatgccccgggaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgaggcatagtcataagcttcatcatcaagatcttgaattgtttgacggagagaatcagcggcgagagcatacttctcggagcaatcttgtaacacctttttaaggacgacgtcgtttgcagcggatgttaggtttccggcgatgtaagttgcggtggaagtggcgtttgtcataccaacgccgatcataatggctgcgagaccttttgtgtcggctgtgggacttcttgggtcggatttgagagccgagacacagaatttgtagttgttcgtggttttgcaagttgattcgattattgtcgcgttgggctagccacaaacagaagcttctaccccggcgtcaatcacgccgtcttcgccgaatttagggactcctttagagatagcttcgatggcttcgggaacatcagcatttaaaatcgttttgtatcttcgactacattggtccaaagcctgttttagttccggtctctttttatatagaccgttgatctcggttaaggtctttgtcgccagcaccttgattttatcgatgaggatgagagcgaggccagagatgtcggcagaggagccacgtgggtcggagttgagtagagagacacaaagtttgaagtctggtgcattgac

SEQ ID NO: 109 - DNA, 1600 bp, Camelina sativa
  misc_feature (383)..(383): n is a, c, g, or t
  misc_feature (522)..(523): n is a, c, g, or t
ctcaaaaattagcattaaaaattctgtaaatgaactttaataaatagtatatatttaattaaaaagcaatattgaaattttgaaaaccaaaaaaatgtatagtaattttgaaattcaaatcattgcaggaaattaaatacatagatggttttaggcataaatacactttccatatcatgatcacttgactaatattaatttggcatatttataatttcatagtaagatgttatttcagtgtggtcacaatattagacattatataatgtatatataatttatattagtgtttttgccaaatttgttcttggatactatagaaactaaaaagattaataacccaaactaaagaaatttaaaaacattcaaattaaattttgatnggacaatatcaatttggtggtatatactaaaataaaagtatattacctgaaaatatcagaaatgatatataggttttttatccttattaagagattttggtaaaggcacgccaccaattcaattatatatatactggtnncgggcagtacacagacaagacacacacacttataaataaacaaaaacgaaacctccatctttttacatataaagatcatcatccaacaagaagaagatgaagatggtcgtgatggttatgatgatgatgatgatgagtgaaggaagtatggtagatcaaacatgtaaacagacaccagacttcaatctctgtgtctctctactcaactccgacccacgtggctcttctgccgacacctctggcctcgctctcatcctcatcgataaaatcaaggtatttttcaattccttttctcatctagtttcttctatatagatattaccaattatctcagattattttcaagtcttattataagaatcaaatcttgactaaaggttttgtggttgttttttaaattatgatattttttctatattattagatgtaatatttaattttattctattctataactttgatctcttaaatttttataaaaaggctcataagtttcgttattctacgaaaaagtaattatcactaagacgtttttgtctataagactataagtaacacaaggggttgtttttgataaataagaagtttttgattacttttgtttagaacacatacctaagcctaagggtgttatttttttttgtgttttcatgtcgtagtaatattgttttcaatttcagtatagtgtatataaagctcgtttgtcgtttctatcccaccaattatgtagctttatttttccagaattatctgaattaaggggagagtttaactacaaataaaaaatgtgaggtaatttctgttgaaatataaacgtatggggttatcttataaatttttttttgtaggttctggcgacaaagaccttaaacgaaatcaacggtctatataaaaagagaccggaactaaaacaggctttagaccaatgtagtcgaagatacaaaacgatcttaaatgctgatgttcccgaagccatcgaagctatctctaaaggagtccctaaatttggcgaagatggtgtgatcgacgccggggtagaagcttctgtttgtgaagaagggtttcaagggaaatctc

SEQ ID NO: 110 - DNA, 1116 bp, Camelina sativa
tacgatggactccagagcggccgcggcgagacggtgaatgaactaatgtgtatatatatgtatgacttactttcgaataatgaactaatgtgtatgtatgacttactttcgaatgaagaaagttagaaagaatacaaattgattcttatttcagttgttcacatgtaaacacgttatatggcatcttgacaaaaagaaatatcacttaattcacattgagaattcttttgttttcatataggactattatatatagcaacaatatgtatcctgtaaatttgaatcccaattgtaacagccatatataatattagcataactattggactaaatgtcatggttaacgtagttaatgtgctattgtaattaattgtcataccacgtaaaaatcaataaaaggtactaaaatcatttcatattttgcaactacaaatgataaacaaaagtagtatttatttttatatatattttaaaatacgtaatatcaagaaactgcttaaaatataagacaagaatcctctttcttccatctctatctctctccgtagacagtttgctcaagcccctcttcttgaaatggcttcttctcttatcttcctcctcctcatctttaccctatcctttccatcctcaaccctaatctcagccaaatccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacttccaccgcaacttacatcgccggaaacctaacatccgctgcaaacgacgtcgtccttaaaaaggtgttacaagattgctccgagaagtatgctctcgccgctgattctctccgtcaaacaattcaatatcttgataatgaagcttatgactatgcttccatgcatgtgctggcggcggaggattatcctaatgtttgccgcaatattttccgccgagctaaggggctgtcttatccggtggagattcgtcggcgtgaacagagtctgagacgtatctgtggtgttgtctcagggattcttgatcgtcttgttgaa

SEQ ID NO: 111 - DNA, 22 bp, Artificial
  Quantitative RT-PCR primer sequence
aacacaaaccacaagaggatca

SEQ ID NO: 112 - DNA, 20 bp, Artificial
  Quantitative RT-PCR primer sequence
cgtcaacgttttcttgtcca

(19) United States
(12) Patent Application Publication
(10) Pub. No.: US 2014/0150135 A1
(43) Pub. Date: May 29, 2014

(54) Synthetic Pathway for Biological Carbon Dioxide Sequestration

(71) Applicant: North Carolina State University, Raleigh, NC (US)

(72) Inventors: Amy Michele Grunden, Holly Springs, NC (US); Heike Inge Ada Sederoff, Raleigh, NC (US)

(73) Assignee: North Carolina State University, Raleigh, NC (US)

(21) Appl. No.: 14/088,142

(22) Filed: Nov. 22, 2013

Related U.S. Application Data
(60) Provisional application No. 61/731,267, filed on Nov. 29, 2012.

(51) Int. Cl. C12N 15/82

(52) U.S. Cl. CPC C12N 15/8261
USPC 800/290; 800/298; 435/419; 554/1; 424/725; 44/307; 426/615; 426/590; 426/635

(57) ABSTRACT

This invention relates to methods for increasing carbon fixation and/or increasing biomass production in a plant, comprising: introducing into a plant, plant part, and/or plant cell one or more heterologous polynucleotides encoding polypeptides having the enzyme activity of succinyl CoA synthetase, 2-oxoglutarate:ferredoxin oxidoreductase, 2-oxoglutarate carboxylase, oxalosuccinate reductase and isocitrate lyase to produce a stably transformed plant, plant part, and/or plant cell expressing the one or more heterologous polynucleotides. The methods further comprise introducing into a plant, plant part or plant cell heterologous polynucleotides encoding polypeptides having the enzyme activity of glyoxylate carboligase and tartronic semialdehyde reductase, and/or heterologous polynucleotides encoding a superoxide reductase from an archaeon species, an aquaporin and/or an inhibitor of cell wall invertase inhibitor. Additionally, transformed plants, plant parts, and/or plant cells are provided as well as products produced from the transformed plants, plant parts, and/or plant cells.

### STATEMENT OF PRIORITY

This application claims the benefit, under 35 U.S.C. §119 (e), of U.S. Provisional Application No. 61/731,267 was filed on Nov. 29, 2012, the entire contents of which is incorporated by reference herein.

### STATEMENT OF GOVERNMENT SUPPORT

This invention was supported in part by funding provided under Grant No 2009-35318-05024 from the United States Department of Agriculture (USDA), and Grant No DE-AR0000207 from the United States Department of Energy (DOE). The United States government has certain rights in this invention.

### STATEMENT REGARDING ELECTRONIC FILING OF A SEQUENCE LISTING

A Sequence Listing in ASCII text format, submitted under 37 C.F.R. §1.821, entitled 5051-812PR_ST25.txt, 314,413 bytes in size, generated on Nov. 22, 2013 and filed via EFS-Web, is provided in lieu of a paper copy. This Sequence Listing is hereby incorporated herein by reference into the specification for its disclosures.

### FIELD OF THE INVENTION

The present invention relates to methods for increasing carbon fixation and biomass production in plants.

### BACKGROUND

All life depends on photosynthetic carbon fixation in which CO₂ is converted to organic compounds in the presence of water and light. However, this is an inefficient process, particularly in C₃ plants, because of a competing process called photorespiration. Photorespiration results in the release of about a quarter of the carbon that is fixed by photosynthesis. The inefficiency of C₃ photosynthesis is largely due to the enzyme ribulose-1,5-bisphosphate carboxylase oxygenase (Rubisco) that catalyzes two competing reactions, carboxylation and oxygenation. Carboxylation leads to net fixed carbon dioxide and oxygenation utilizes oxygen and results in a net loss of carbon. The relative concentrations of carbon dioxide and oxygen and the temperature as well as water availability determine which reaction occurs or dominates. Thus, C3 plants do not grow efficiently in hot and/or dry areas because, as the temperature increases, Rubisco incorporates more oxygen. Some plants such as C4 and CAM (Crassulacean acid metabolism) plants have developed mechanisms that reduce the effect of photorespiration by more efficiently delivering carbon dioxide to Rubisco, thereby outcompeting the oxygenase activity.

### SUMMARY OF THE INVENTION

This invention is directed to methods for improving the efficiency of CO₂ fixation and increasing biomass production in plants.

Thus, in one aspect, the present invention provides a method for increasing carbon fixation and/or increasing biomass production in a plant, comprising: introducing into a plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase and (e) isocitrate lyase to produce a stably transformed plant, plant part, and/or plant cell expressing said one or more heterologous polynucleotides.

In another aspect of the invention, the method further comprises introducing into the plant, plant part, and/or plant cell one or more heterologous polynucleotides encoding polypeptides having the enzyme activity of a glyoxylate carboligase and a tartronic semialdehyde reductase to produce a stably transformed plant, plant part, and/or plant cell expressing said one or more heterologous polynucleotides.

In a further aspect of the invention, the method further comprises introducing into the plant, plant part, and/or plant cell a heterologous polynucleotide encoding a superoxide reductase (SOR) from an archaeon species to produce a stably transformed plant, plant part and/or plant cell expressing said heterologous polynucleotide.

In additional aspects of the invention, the method further comprises introducing into the plant, plant part, and/or plant cell a heterologous polynucleotide encoding an aquaporin to produce a stably transformed plant, plant part, and/or plant cell expressing said heterologous polynucleotide.

In a further aspect, the present invention provides a stably transformed plant, plant part and/or plant cell, comprising one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase and (e) isocitrate lyase. In other aspects, said stably transformed plant, plant part and/or plant cell further comprises one or more heterologous polynucleotides encoding polypeptides having the enzyme activity of a glyoxylate carboligase and a tartronic semialdehyde reductase, a heterologous polynucleotide encoding a superoxide reductase from an archaeon species and/or a heterologous polynucleotide encoding an aquaporin.

In additional aspects, the present invention provides crops produced from the stably transformed plants of the invention as well as products produced from the transformed plants, plant parts and/or plant cells of this invention.

The foregoing and other objects and aspects of the present invention are explained in detail in the drawings and specification set forth below.

### BRIEF DESCRIPTION OF THE DRAWINGS

FIG. 1 shows a schematic for the condensed reverse tricarboxylic acid (crTCA) cycle.

FIG. 2 shows a schematic view of 2-oxoglutarate:ferredoxin oxidoreductase (OGOR) enzyme assay.

FIG. 3 shows a schematic view of reductive carboxylation catalyzed by 2-oxoglutarate carboxylase/isocitrate dehydrogenase (OGC/ICDH) (adapted from Aoshima et al. Mol. Microbiol. 62:748-759 (2006)).

FIG. 4 shows purified recombinant enzymes for crTCA cycle enzyme steps 1-3 (succinyl CoA synthetase (ScS), 2-oxoglutarate ferredoxin oxidoreductase (KOR), and 2-oxoglutarate carboxylase (OGC)) on an SDS-polyacrylamide gel.

FIG. 5 shows purified recombinant enzymes for crTCA cycle enzyme step 4 (oxalosuccinate reductase (ICDH)) and step 5 (isocitrate lyase (ICL)) on an SDS-polyacrylamide gel.

FIG. 6 provides a spectrum showing the succinyl CoA synthetase (SCS) assay. For the SCS assay spectra, change in absorbance at 230 nm is indicated on the Y axis versus time (min) on the X-axis. The different colored spectra traces correspond to SCS assay repeats.

FIG. 7 shows a schematic view of the coupled OGC-PK-LDH assay used to determine the rate of ATP hydrolysis by OGC. OGC is 2-oxoglutarate carboxylase, PK is pyruvate kinase and LDH is lactate dehydrogenase.

FIG. 8 provides a spectrum showing the coupled 2-oxoglutarate carboxylase (OGC) assay spectra. Change in absorbance at 340 nm is indicated on the Y axis versus time (min) on the X-axis. The different colored spectra traces correspond to OGC assay repeats.

FIG. 9 provides a spectrum showing an oxalosuccinate reductase (isocitrate dehydrogenase, ICDH) assay for ICDH from Nitrosococcus halophilus Nc4. For the ICDH assay spectra, change in absorbance at 340 nm is indicated on the Y axis versus time (min) on the X-axis. The different colored spectra traces correspond to ICDH assay repeats.

FIG. 10 provides a spectrum showing an isocitrate lyase (ICL) assay from Rhodococcus pyridinivorans AK37. For the ICL assay spectra, change in absorbance at 324 nm is indicated on the Y axis versus time (min) on the X-axis. The different colored spectra traces correspond to ICL assay repeats.

FIG. 11 shows expression of both cell wall invertase isoforms from C. sativa in both seeds and young leaves.

FIG. 12 shows an agarose gel with repeated TAIL-PCR results for two different primary dilution rates. LAD=arbitrary degenerate primer. N2=secondary PCR product. N3=tertiary PCR product. Arrows indicate bands that were re-amplified and extracted for sequencing. Light and dark arrows correspond to CWII1 and CWII2 respectively, including their respective upstream regions.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, refers to variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The terms “increase,” “increasing,” “increased,” “enhance,” “enhanced,” “enhancing,” and “enhancement” (and grammatical variations thereof), as used herein, describe an elevation in, for example, carbon fixation and/or biomass production, and/or an elevation in CO₂ uptake in a plant, plant part or plant cell. This increase can be observed by comparing the increase in the plant, plant part or plant cell transformed with, for example, one or more heterologous polynucleotides encoding polypeptides having the enzyme activity of succinyl CoA synthetase, 2-oxoglutarate:ferredoxin oxidoreductase, 2-oxoglutarate carboxylase, oxalosuccinate reductase and isocitrate lyase and a heterologous polynucleotide encoding an aquaporin to the appropriate control (e.g., the same organism lacking (i.e., not transformed with) said heterologous polynucleotides). Thus, as used herein, the terms “increase,” “increasing,” “increased,” “enhance,” “enhanced,” “enhancing,” and “enhancement” (and grammatical variations thereof), and similar terms indicate an elevation of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control (e.g., a plant, plant part and/or plant cell that does not comprise said heterologous polynucleotide).

As used herein, the terms “reduce,” “reduced,” “reducing,” “reduction,” “diminish,” “suppress,” and “decrease” (and grammatical variations thereof), describe, for example, a decrease in the reactive oxygen species in a plant, plant cell and/or plant part as compared to a control as described herein. Thus, as used herein, the terms “reduce,” “reduces,” “reduced,” “reduction,” “diminish,” “suppress,” and “decrease” and similar terms mean a decrease of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or any range therein, as compared to a control (e.g., a plant, plant part and/or plant cell that does not comprise a heterologous polynucleotide encoding SOR from an archaeon species).

As used herein, the terms “express,” “expresses,” “expressed” or “expression,” and the like, with respect to a nucleotide sequence (e.g., RNA or DNA) indicates that the nucleotide sequence is transcribed and, optionally, translated. Thus, a nucleotide sequence may express a polypeptide of interest or a functional untranslated RNA. A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers, and the like.

Accordingly, the present invention is directed to compositions and methods for increasing carbon fixation and biomass production in a plant, plant cell and/or plant part by introducing in the plant, plant cell and/or plant part heterologous polynucleotides that encode polypeptides for a synthetic condensed reverse tricarboxylic acid (crTCA) cycle described herein. The invention can further comprise introducing into the plant, plant part and/or plant cell additional heterologous polynucleotides encoding additional useful polypeptides or functional nucleic acids. Thus, for example in some embodiments, heterologous polynucleotides encoding polypeptides that feed the products of the crTCA cycle of this invention into the Calvin Benson cycle can be introduced into the plant, plant part and/or plant cell of the invention. In other embodiments, heterologous polynucleotides encoding superoxide reductase, heterologous polynucleotides encoding aquaporin, and/or heterologous polynucleotides encoding functional nucleic acids, including but not limited to an RNAi that inhibits cell wall invertase inhibitor activity, can also be introduced into a plant, plant part, or plant cell of the invention.

Thus, a first aspect of the present invention provides a method for increasing carbon fixation and/or increasing biomass production in a plant, comprising, consisting essentially of, or consisting of: introducing into a plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase and (e) isocitrate lyase to produce a stably transformed plant, plant part, and/or plant cell expressing said one or more heterologous polynucleotides to produce said polypeptides, wherein the expression of the one or more heterologous polynucleotides results in the plant, plant part and/or plant cell having increased carbon fixation and/or increased biomass production as compared to a plant, plant part and/or plant cell not transformed with and stably expressing said heterologous polynucleotides. In some aspects, the method further comprises regenerating a stably transformed plant or plant part from the stably transformed plant cell, wherein expression of the one or more heterologous polynucleotides results in the stably transformed plant and/or plant part having increased carbon fixation and/or increased biomass production as compared to a control (e.g., a plant or plant part not transformed with and stably expressing said heterologous polynucleotides).

“Increased biomass production” as used herein refers to a transformed plant or plant part having a greater dry weight over the entire plant or any organ of the plant (leaf, stem, roots, seeds, seed pods, flowers, etc), increased plant height, leaf number, and/or seed number or increased root volume compared to the native or wild type (e.g., a plant, plant part that is not transformed with the heterologous polynucleotides of the invention (e.g., heterologous polynucleotides encoding polypeptides having the enzyme activity of succinyl CoA synthetase, 2-oxoglutarate:ferredoxin oxidoreductase, 2-oxoglutarate carboxylase, oxalosuccinate reductase, isocitrate lyase, glyoxylate carboligase, tartronic semialdehyde reductase, heterologous polynucleotides encoding SOR, an aquaporin, an inhibitor of cwII, and the like). Increased biomass can also refer to a greater dry weight of cells (e.g., tissue culture, cell suspension (e.g., algal culture), and the like) as compared to cells not transformed with the heterologous polynucleotides of the invention.

“Increased carbon fixation” as used herein refers to a greater conversion of CO₂ to organic carbon compounds in a transgenic plant (e.g., a plant, plant part that is not transformed with the heterologous polynucleotides of the invention (e.g., heterologous polynucleotides encoding polypeptides having the enzyme activity of encoding succinyl CoA synthetase, 2-oxoglutarate:ferredoxin oxidoreductase, 2-oxoglutarate carboxylase, oxalosuccinate reductase, isocitrate lyase, glyoxylate carboligase, tartronic semialdehyde reductase, heterologous polynucleotides encoding SOR, an aquaporin, an inhibitor of cwII, and the like)) when compared to the native or wild type (e.g., not transformed with said heterologous polynucleotides. “Increased carbon fixation” can be measured by analyzing CO₂ fixation rates using a Licor System or radiolabeled ¹⁴CO₂ or by quantifying dry biomass. Increased carbon fixation can also occur for transformed cells (e.g., tissue culture, cell suspension (e.g., algal culture), and the like) as compared to cells not transformed with the heterologous polynucleotides of the invention.

The polypeptides succinyl CoA synthetase, 2-oxoglutarate:ferredoxin oxidoreductase, 2-oxoglutarate carboxylase, oxalosuccinate reductase and isocitrate lyase (i.e., the enzymes of the synthetic crTCA cycle of the invention), and the polynucleotides that encode said polypeptides are known in the art and are produced by many different organisms. Selection of a particular polypeptide for use with this invention is based on a number of factors including, for example, the number of subunits in the enzyme (e.g., selecting those with the fewest number of subunits) and the kinetic properties of the individual polypeptides (e.g., a polypeptide with a high kcat value). Examples of organisms from which these polypeptides and polynucleotides can be derived include, but are not limited to, Escherichia coli (e.g., E. coli MG1655), Azotobacter vinelandii (e.g., A. vinelandii DJ), Bradyrhizobium sp. (e.g., Bradyrhizobium sp. BTAi1), Azospirillum sp (e.g., Azospirillum sp. B510), Paenibacillus sp. (e.g. Paenibacillus sp. JDR-2), Halobacterium sp. (e.g., Halobacterium sp NRC-1), Hydrogenobacter thermophilus (e.g., H. thermophilus TK-6), Bacillus sp (e.g., Bacillus sp M3-13), Paenibacillus larvae subsp. larvae (e.g., Paenibacillus larvae subsp. larvae B-3650), Haladaptus paucihalophilus (e.g., H. paucihalophilus DX253), Magnetococcus sp. (e.g., Magnetococcus sp. MC-1), Candidatus Nitrospira defluvii (e.g., Candidatus Nitrospira defluvii NIDE1204), Thiocystis violascens (e.g., T. violascens DSM198), Mariprofundus ferroxydans (e.g., M. ferroxydans PV-1), Pseudomonas stutzeri (e.g., P. stutzeri ATCC14405), Acinetobacter baumannii (e.g. A. baumannii ABT07, A. baumannii ACICU), Chlorobium limicola (e.g. C. limicola DSM 245), Kosmotoga olearia (e.g. K. olearia TBF 19.5.1), Marine gamma proteobacterium (e.g. Marine gamma proteobacterium HTCC2080), Corynebacterium glutamicum (e.g. C. glutamicum ATCC 13032), Gordonia alkanivorans (e.g. G. alkanivorans NBRC 16433), Nocardia farcinica (e.g. N. farcinica IFM 10152), Rhodococcus pyridinivorans (e.g. R. pyridinivorans AK37), and Rhodococcus jostii (e.g. R. jostii RHA1).

Thus, in some embodiments, a polypeptide and/or polynucleotide encoding a polypeptide having the enzyme activity of succinyl CoA synthetase can be from Escherichia coli, Azotobacter vinelandii, Bradyrhizobium sp., Azospirillum sp., or any combination thereof. In some embodiments, the polypeptide having the enzyme activity of succinyl CoA synthetase can be a two subunit enzyme. In other embodiments, a polypeptide and/or polynucleotide encoding a polypeptide having the enzyme activity of 2-oxoglutarate:ferredoxin oxidoreductase can be from Paenibacillus sp., Halobacterium sp., Hydrogenobacter thermophilus, Bacillus sp, Paenibacillus larvae subsp. larvae, Haladaptus paucihalophilus, Magnetococcus sp., or any combination thereof. In further embodiments, the polypeptide having the enzyme activity of 2-oxoglutarate:ferredoxin oxidoreductase can be a two subunit enzyme. In still other embodiments, a polypeptide and/or polynucleotide encoding a polypeptide having the enzyme activity of 2-oxoglutarate carboxylase can be from Candidatus Nitrospira defluvii, Hydrogenobacter thermophilus, Thiocystis violascens, Mariprofundus ferroxydans, Pseudomonas stutzeri, or any combination thereof. In some embodiments, the polypeptide having the enzyme activity of 2-oxoglutarate carboxylase can be a two subunit enzyme. In additional embodiments, a polypeptide and/or polynucleotide encoding a polypeptide having the enzyme activity of oxalosuccinate reductase can be from Acinetobacter baumannii, Chlorobium limicola, Kosmotoga olearia, Marine gamma proteobacterium, or any combination thereof. In further embodiments, a polypeptide and/or polynucleotide encoding a polypeptide having the enzyme activity of isocitrate lyase can be from Corynebacterium glutamicum, Gordonia alkanivorans, Nocardia farcinica, Rhodococcus pyridinivorans, Rhodococcus jostii, or any combination thereof.

More particularly, in some embodiments, a polynucleotide encoding a polypeptide having the enzyme activity of succinyl CoA synthetase useful with this invention includes, but is not limited to, a nucleotide sequence from E. coli strain K-12 substr. MG1655 (e.g., NCBI Accession Nos. NC_{—}000913.2 (772,237 . . . 763,403), NC_{—}000913.2 (763,403 . . . 764,272); see, e.g., SEQ ID NO:3); from Azotobacter vinelandii DJ (e.g., NCBI Accession Nos. NC_{—}012560.1 (3,074,152 . . . 3,075,321), NC_{—}012560.1 (3,073,268 . . . 3,074,155); see, e.g., SEQ ID NO:6); from Bradyrhizobium sp. BTAi1 (e.g., NCBI Accession Nos. NC_{—}009485.1 (393,292 . . . 394,488), NC_{—}009485.1 (394,545 . . . 395,429); see, e.g., SEQ ID NO:9); and/or from Azospirillum sp. B510 (e.g., NCBI Accession Nos. NC_{—}013854.1 (2,941,010 . . . 2,942,206), NC_{—}013854.1 (2,942,208 . . . 2,943,083); see, e.g., SEQ ID NO:12). In other embodiments, a polypeptide having the enzyme activity of succinyl CoA synthetase can have an amino acid sequence that includes but is not limited to an amino acid sequence from E. coli strain K-12 substr. MG1655 (e.g., NCBI Accession Nos. NP_{—}415256.1 and NP_{—}415257.1); see, e.g., SEQ ID NO:1 and SEQ ID NO:2); from Azotobacter vinelandii DJ (e.g., NCBI Accession Nos. YP_{—}002800115.1 and YP_{—}002800114.1); see, e.g., SEQ ID NO:4 and SEQ ID NO:5); from Bradyrhizobium sp.BTAi1 (e.g., NCBI Accession Nos. YP_{—}001236586.1 and YP_{—}001236587.1); see, e.g., SEQ ID NO:7 and SEQ ID NO:8); and/or from Azospirillum sp. B510 (e.g., NCBI Accession Nos. YP_{—}003449758.1 and YP_{—}003449759.1); see, e.g., SEQ ID NO:10 and SEQ ID NO:11. In some embodiments, a heterologous polynucleotide encoding a polypeptide having the enzyme activity of succinyl CoA synthetase can be from E. coli strain K-12 substr. MG1655. In some particular embodiments, a heterologous polynucleotide encoding a polypeptide having the enzyme activity of succinyl CoA synthetase from E. coli strain K-12 substr. MG1655 comprises, consists essentially of, or consists of the nucleotide sequence of SEQ ID NO:3.

In other embodiments, a polynucleotide encoding a polypeptide having the enzyme activity of 2-oxoglutarate:ferredoxin oxidoreductase useful with this invention includes, but is not limited to, a nucleotide sequence from Halobacterium sp. NRC-1 (e.g., NCBI Accession Nos. NC_{—}002607.1 (856,660 . . . 858,582), NC_{—}002607.1 (855,719 . . . 856,657); see, e.g., SEQ ID NO:15); from Hydrogenobacter thermophilus TK-6 (e.g., NCBI Accession Nos. NC_{—}013799.1 (997,525 . . . 999,348), NC_{—}013799.1 (996,624 . . . 997,511); see, e.g., SEQ ID NO:18); from Bacillus sp. M3-13 (e.g., NCBI Accession Nos. NZ_ACPC01000013.1 (932 . . . 2,668), NZ_ACPC01000013.1 (65 . . . 931); see, e.g., SEQ ID NO:21); from Paenibacillus larvae subsp. larvae B-3650 (e.g., NCBI Accession Nos. NZ_ADZY02000226.1 (7,939 . . . 9,687), NZ_ADZY02000226.1 (7,085 . . . 7,951); see, e.g., SEQ ID NO:24); from Haladaptatus paucihalophilus DX253 (e.g., NCBI Accession Nos. NZ_AEMG01000009.1 (157,678 . . . 159,432), NZ_AEMG01000009.1 (156,818 . . . 157,681); see, e.g., SEQ ID NO:27); and/or from Magnetococcus sp. MC-1 (e.g., NCBI Accession Nos. NC_{—}008576.1 (2,161,258 . . . 2,162,979), NC_{—}008576.1 (2,162,976 . . . 2,163,854); see, e.g., SEQ ID NO:30). In other embodiments, a polypeptide having the enzyme activity of 2-oxoglutarate:ferredoxin oxidoreductase can have an amino acid sequence that includes, but is not limited to, an amino acid sequence from Halobacterium sp. NRC-1 (e.g., NCBI Accession Nos. AAG19514.1, AAG19513.1, NP_{—}280034.1 and NP_{—}280033.1); see, e.g., SEQ ID NO:13 and SEQ ID NO:14); from Hydrogenobacter thermophilus TK-6 (e.g., NCBI Accession Nos. YP_{—}003432752.1 and YP_{—}003432751.1); see, e.g., SEQ ID NO:16 and SEQ ID NO:17); from Bacillus sp. M3-13 (e.g., NCBI Accession Nos. ZP_{—}07708142.1 and ZP_{—}07708141.1); see, e.g., SEQ ID NO:19 and SEQ ID NO:20); from Paenibacillus larvae subsp. larvae B-3650 (e.g., NCBI Accession Nos. ZP_{—}09070120.1 and ZP_{—}09070119.1); see, e.g., SEQ ID NO:22 and SEQ ID NO:23); from Haladaptatus paucihalophilus DX253 (e.g., NCBI Accession Nos. ZP_{—}08044530.1 and ZP_{—}08044529.1); see, e.g., SEQ ID NO:25 and SEQ ID NO:26); and/or from Magnetococcus sp. MC-1 (e.g., NCBI Accession Nos. YP_{—}865663.1 and YP_{—}865664.1); see, e.g., SEQ ID NO:28 and SEQ ID NO:29). In some embodiments, a heterologous polynucleotide encoding a polypeptide having the enzyme activity of 2-oxoglutarate:ferredoxin oxidoreductase can be from Paenibacillus sp. subsp. larvae B-3650. In particular embodiments, a heterologous polynucleotide encoding a polypeptide having the enzyme activity of 2-oxoglutarate:ferredoxin oxidoreductase from Paenibacillus sp. subsp. larvae B-3650 comprises, consists essentially of, or consists of the nucleotide sequence of SEQ ID NO:24.

In further embodiments, a polynucleotide encoding a polypeptide having the enzyme activity of 2-oxoglutarate carboxylase useful with this invention includes, but is not limited to, a nucleotide sequence from Hydrogenobacter thermophilus TK-6 (e.g., NCBI Accession Nos. NC_{—}013799.1 (1,271,487 . . . 1,273,445), NC_{—}013799.1 (1,273,469 . . . 1,274,887); see, e.g., SEQ ID NO:33); from Candidatus Nitrospira defluvii (e.g., NCBI Accession Nos. NC_{—}014355.1 (1,174,721 . . . 1,176,652), NC_{—}014355.1 (1,176,781 . . . 1,178,199); see, e.g., SEQ ID NO:36); from Hydrogenobacter thermophilus TK-6 (e.g., NCBI Accession Nos. NC_{—}013799.1 (1,271,487 . . . 1,273,445), NC_{—}013799.1 (1,273,469 . . . 1,274,887); see, e.g., SEQ ID NO:39); from Thiocystis violascens DSM198 (e.g., NCBI Accession Nos. NZ_AGFC01000013.1 (61,879 . . . 63,297) and (63,889 . . . 65,718); see, e.g., SEQ ID NO:42); from Mariprofundus ferrooxydans PV-1 (e.g., NCBI Accession Nos. NZ_AATS01000007.1 (81,967 . . . 83,385) and (83,475 . . . 85,328); see, e.g., SEQ ID NO:45); and/or from Pseudomonas stutzeri ATCC14405 (AGSL01000085.1 (52,350 . . . 53,765) and (50,522 . . . 52,339); see, e.g., SEQ ID NO:48). In further embodiments, a polypeptide having the enzyme activity of 2-oxoglutarate carboxylase can have an amino acid sequence that includes, but is not limited to, an amino acid sequence from Hydrogenobacter thermophilus TK-6 (e.g., NCBI Accession Nos. YP_{—}003433044.1 and YP_{—}003433045.1); see, e.g., SEQ ID NO:31 and SEQ ID NO:32); from Candidatus Nitrospira defluvii (e.g., NCBI Accession Nos. YP_{—}003796887.1 and YP_{—}003796888.1); see, e.g., SEQ ID NO:34 and SEQ ID NO:35); from Hydrogenobacter thermophilus TK-6 (e.g., NCBI Accession Nos. YP_{—}003433044.1 and YP_{—}003433045.1); see, e.g., SEQ ID NO:37 and SEQ ID NO:38); from Thiocystis violascens DSM198 (e.g., NCBI Accession Nos. ZP_{—}08925050.1 and ZP_{—}08925052.1); see, e.g., SEQ ID NO:40 and SEQ ID NO:41 and/or SEQ ID NO:43 and SEQ ID NO:44); from Mariprofundus ferrooxydans PV-1 (e.g., NCBI Accession Nos. ZP_{—}01452577.1 and ZP_{—}01452578.1); see, e.g., SEQ ID NO:46 and SEQ ID NO:47); and/or from Pseudomonas stutzeri ATCC14405 (e.g., NCBI Accession Nos. EHY78621.1 and EHY78620.1); see, e.g., SEQ ID NO:49 and SEQ ID NO:50). In some embodiments, a heterologous polynucleotide encoding a polypeptide having the enzyme activity of a 2-oxoglutarate carboxylase can be a 2-oxoglutarate carboxylase from Candidatus Nitrospira defluvii. In some particular embodiments, a heterologous polynucleotide encoding a polypeptide having the enzyme activity of 2-oxoglutarate carboxylase from Candidatus Nitrospira defluvii comprises, consists essentially of, or consists of the nucleotide sequence of SEQ ID NO:36. In other embodiments, a heterologous polynucleotide encoding a polypeptide having the enzyme activity of a 2-oxoglutarate carboxylase can be a 2-oxoglutarate carboxylase from Hydrogenobacter thermophilus TK-6. In some particular embodiments, a heterologous polynucleotide encoding a polypeptide having the enzyme activity of 2-oxoglutarate carboxylase from Hydrogenobacter thermophilus TK-6 comprises, consists essentially of, or consists of the nucleotide sequence of SEQ ID NO:33, SEQ ID NO:39 and/or SEQ ID NO:42.

In still further embodiments, a polynucleotide encoding a polypeptide having the enzyme activity of oxalosuccinate reductase useful with this invention includes, but is not limited to, a polynucleotide from Chlorobium limicola DSM 245 (e.g., NCBI Accession Nos. AB076021.1); see, e.g., SEQ ID NO:53); from Kosmotoga olearia TBF 19.5.1 (e.g., NCBI Accession Nos. NC_{—}012785.1 (1,303,493 . . . 1,304,695); see, e.g., SEQ ID NO:55); from Acinetobacter baumannii ACICU (e.g., NCBI Accession Nos. NC_{—}010611.1 (2,855,563 . . . 2,856,819); see, e.g., SEQ ID NO:57); from Marine gamma proteobacterium HTCC2080 (e.g., NCBI Accession Nos. NZ_AAVV01000002.1 (123,681 . . . 124,934); see, e.g., SEQ ID NO:59); and/or from Nitrosococcus halophilus Nc4 (e.g., NCBI Accession Nos. NC_{—}013960.1 (2,610,547 . . . 2,611,815); see, e.g., SEQ ID NO:61). In other embodiments, a polypeptide having the enzyme activity of oxalosuccinate reductase can have an amino acid sequence that includes, but is not limited to, an amino acid sequence from Chlorobium limicola DSM 245 (e.g., NCBI Accession Nos. BAC00856.1); see, e.g., SEQ ID NO:52); from Kosmotoga olearia TBF 19.5.1 (e.g., NCBI Accession Nos. YP_{—}002940928.1); see, e.g., SEQ ID NO:54); from Acinetobacter baumannii ACICU (e.g., NCBI Accession Nos. YP_{—}001847346.1); see, e.g., SEQ ID NO:56); from Marine gamma proteobacterium HTCC2080 (e.g., NCBI Accession Nos. ZP_{—}01625318.1); see, e.g., SEQ ID NO:58); and/or from Nitrosococcus halophilus Nc4 (e.g., NCBI Accession Nos. YP_{—}003528006.1); see, e.g., SEQ ID NO:60). In some embodiments, a heterologous polynucleotide encoding a polypeptide having the enzyme activity of an oxalosuccinate reductase can be from Acinetobacter baumannii. In some particular embodiments, a heterologous polynucleotide encoding a polypeptide having the enzyme activity of succinyl CoA synthetase from Acinetobacter baumannii comprises, consists essentially of, or consists of nucleotide sequence of SEQ ID NO:57. In other embodiments, a heterologous polynucleotide encoding a polypeptide having the enzyme activity of an oxalosuccinate reductase can be from Chlorobium limicola. In some particular embodiments, a heterologous polynucleotide encoding a polypeptide having the enzyme activity of succinyl CoA synthetase from Chlorobium limicola comprises, consists essentially of, or consists of nucleotide sequence of SEQ ID NO:53. In further embodiments, a heterologous polynucleotide encoding a polypeptide having the enzyme activity of an oxalosuccinate reductase can be from Kosmotoga olearia TBF 19.5.1. In some particular embodiments, a heterologous polynucleotide encoding a polypeptide having the enzyme activity of succinyl CoA synthetase from Kosmotoga olearia TBF 19.5.1 comprises, consists essentially of, or consists of nucleotide sequence of SEQ ID NO:55. In still further embodiments, a heterologous polynucleotide encoding a polypeptide having the enzyme activity of an oxalosuccinate reductase can be from Nitrosococcus halophilus Nc4. In some particular embodiments, a heterologous polynucleotide encoding a polypeptide having the enzyme activity of succinyl CoA synthetase from Nitrosococcus halophilus Nc4 comprises, consists essentially of, or consists of nucleotide sequence of SEQ ID NO:60.

In additional embodiments, a polynucleotide encoding a polypeptide having the enzyme activity of isocitrate lyase useful with this invention includes, but is not limited to, a polynucleotide from Corynebacterium glutamicum ATCC 13032 (e.g., NCBI Accession Nos. NC_{—}003450.3 (2,470,741 . . . 2,472,039); see, e.g., SEQ ID NO:63); from Gordonia alkanivorans NBRC 16433 (e.g., NCBI Accession Nos. NZ_BACI01000050.1 (37,665 . . . 38,960); see, e.g., SEQ ID NO:65); Nocardia farcinica IFM 10152 (e.g., NCBI Accession Nos. NC_{—}006361.1 (5,525,226 . . . 5,526,515); see, e.g., SEQ ID NO:67); that from Rhodococcus pyridinivorans AK37 (e.g., NCBI Accession Nos. NZ_AHBW01000053.1 (20,169 . . . 21,458); see, e.g., SEQ ID NO:69); and/or from Rhodococcus jostii RHA1 (e.g., NCBI Accession Nos. NC_{—}008268.1 (2,230,309 . . . 2,231,598); see, e.g., SEQ ID NO:71). In other embodiments, a polypeptide having the enzyme activity of isocitrate lyase can have an amino acid sequence that includes, but is not limited to, an amino acid sequence from Corynebacterium glutamicum ATCC 13032 (e.g., NCBI Accession Nos. NP_{—}601531.1); see, e.g., SEQ ID NO:62); from Gordonia alkanivorans NBRC 16433 (e.g., NCBI Accession Nos. ZP_{—}08765259.1); see, e.g., SEQ ID NO:64); Nocardia farcinica IFM 10152 (e.g., NCBI Accession Nos. YP_{—}121446.1); see, e.g., SEQ ID NO:66); that from Rhodococcus pyridinivorans AK37 (e.g., NCBI Accession Nos. ZP_{—}09310682.1); see, e.g., SEQ ID NO:68); and that from Rhodococcus jostii RHA1 (e.g., NCBI Accession Nos. YP_{—}702087.1); see, e.g., SEQ ID NO:70). In some embodiments, a heterologous polynucleotide encoding a polypeptide having the enzyme activity of isocitrate lyase can be from Corynebacterium glutamicum. In some particular embodiments, a heterologous polynucleotide encoding a polypeptide having the enzyme activity of an isocitrate lyase from Corynebacterium glutamicum comprises, consists essentially of, or consists of nucleotide sequence of SEQ ID NO:63. In further embodiments, a heterologous polynucleotide encoding a polypeptide having the enzyme activity of isocitrate lyase can be from Rhodococcus pyridinivorans AK37. In some particular embodiments, a heterologous polynucleotide encoding a polypeptide having the enzyme activity of an isocitrate lyase from Rhodococcus pyridinivorans AK37 comprises, consists essentially of, or consists of nucleotide sequence of SEQ ID NO:68.

In further embodiments, polypeptides and the polynucleotides encoding said polypeptides can be modified for use with this invention. For example, a native or wild type intergenic spacer sequence in a selected polynucleotide can be substituted with another known spacer or a synthetic spacer sequence. Thus, for example, the intergenic spacer sequence in the 2-oxoglutarate carboxylase polynucleotide sequence from Candidatus Nitrospira defluvii and/or Thiocystis violascens DSM198 can be substituted with the 26 base pair spacer from the 2-oxoglutarate carboxylase Hydrogenobacter thermophilus polynucleotide sequence (see, e.g., the spacer sequence in SEQ ID NO:33) resulting in a 2-oxoglutarate carboxylase polypeptide having the nucleotide sequence of SEQ ID NO: 36 or SEQ ID NO:45, respectively.

Other modifications of polypeptides useful with this invention include amino acid substitutions (and the corresponding base pair changes in the respective polynucleotide encoding said polypeptide). Thus, in some embodiments, a polypeptide and/or polynucleotide sequence of the invention can be a conservatively modified variant. As used herein, “conservatively modified variant” refers to polypeptide and polynucleotide sequences containing individual substitutions, deletions or additions that alter, add or delete a single amino acid or nucleotide or a small percentage of amino acids or nucleotides in the sequence, where the alteration results in the substitution of an amino acid with a chemically similar amino acid. Conservative substitution tables providing functionally similar amino acids are well known in the art.

As used herein, a conservatively modified variant of a polypeptide is biologically active and therefore possesses the desired activity of the reference polypeptide (e.g., succinyl CoA synthetase, 2-oxoglutarate:ferredoxin oxidoreductase, 2-oxoglutarate carboxylase, oxalosuccinate reductase, isocitrate lyase, SOR, aquaporin and the like) as described herein. The variant can result from, for example, a genetic polymorphism or human manipulation. A biologically active variant of the reference polypeptide can have at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more sequence identity (e.g., about 30% to about 99% or more sequence identity and any range therein) to the amino acid sequence for the reference polypeptide as determined by sequence alignment programs and parameters described elsewhere herein. An active variant can differ from the reference polypeptide sequence by as few as 1-15 amino acid residues, as few as 1-10, such as 6-10, as few as 5, as few as 4, 3, 2, or even 1 amino acid residue.

Naturally occurring variants may exist within a population. Such variants can be identified by using well-known molecular biology techniques, such as the polymerase chain reaction (PCR), and hybridization as described below. Synthetically derived nucleotide sequences, for example, sequences generated by site-directed mutagenesis or PCR-mediated mutagenesis which still encode a polypeptide of the invention, are also included as variants. One or more nucleotide or amino acid substitutions, additions, or deletions can be introduced into a nucleotide or amino acid sequence disclosed herein, such that the substitutions, additions, or deletions are introduced into the encoded protein. The additions (insertions) or deletions (truncations) may be made at the N-terminal or C-terminal end of the native protein, or at one or more sites in the native protein. Similarly, a substitution of one or more nucleotides or amino acids may be made at one or more sites in the native protein.

For example, conservative amino acid substitutions may be made at one or more predicted, preferably nonessential amino acid residues. A “nonessential” amino acid residue is a residue that can be altered from the wild-type sequence of a protein without altering the biological activity, whereas an “essential” amino acid is required for biological activity. A “conservative amino acid substitution” is one in which the amino acid residue is replaced with an amino acid residue with a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, where such residues are essential for protein activity.

In some embodiments, amino acid changes can be made to alter the catalytic activity of an enzyme. For example, amino acid substitutions can be made to a thermoactive enzyme that has little activity at room temperature (e.g., about 20° C. to about 50° C.) so as to increase activity at these temperatures. A comparison can be made between the thermoactive enzyme and a mesophilic homologue having activity at the desired temperatures. This can provide discrete differences in amino acids that can then be the focus of amino acid substitutions.

Thus, in some embodiments, amino acid sequence variants of a reference polypeptide can be prepared by mutating the nucleotide sequence encoding the enzyme. The resulting mutants can be expressed recombinantly in plants, and screened for those that retain biological activity by assaying for the enzyme activity (e.g., succinyl CoA synthetase, 2-oxoglutarate:ferredoxin oxidoreductase, 2-oxoglutarate carboxylase, oxalosuccinate reductase, isocitrate lyase, SOR, aquaporin activity and the like) using standard assay techniques as described herein. Methods for mutagenesis and nucleotide sequence alterations are known in the art. See, e.g., Kunkel (1985) Proc. Natl. Acad. Sci. USA 82:488-492; Kunkel et al. (1987) Methods in Enzymol. 154:367-382; and Techniques in Molecular Biology (Walker & Gaastra eds., MacMillan Publishing Co. 1983) and the references cited therein; as well as U.S. Pat. No. 4,873,192. Clearly, the mutations made in the DNA encoding the variant must not disrupt the reading frame and preferably will not create complementary regions that could produce secondary mRNA structure. See, EP Patent Application Publication No. 75,444. Guidance as to appropriate amino acid substitutions that do not affect biological activity of the protein of interest may be found in the model of Dayhoff et al. (1978) Atlas of Protein Sequence and Structure (National Biomedical Research Foundation, Washington, D.C.).

In a representative embodiment, the large subunit from the 2-oxoglutarate carboxylase polypeptide (cfiA) from Hydrogenobacter thermophilus TK-6 can be modified at residue 203 to be alanine (A) instead of methionine (M), at residue 205 to be valine (V) instead of phenylalanine (F), at residue 234 to be methionine (M) instead of threonine (T), at residue 236 to be isoleucine (I) instead of threonine (T), at residue 240 to be leucine (L) instead of methionine (M), at residue 274 to be arginine (R) instead of glutamic acid (E) and for at residue 288 to be glutamine (Q) instead of aspartic acid (D) as shown, for example, in the amino acid sequences of SEQ ID NO:38 and SEQ ID NO:41 and the corresponding codon changes as shown, for example, in the nucleotide sequences of SEQ ID NO:39 or SEQ ID NO:42. Such changes result in a thermophilic 2-oxoglutarate carboxylase that can function at lower temperatures than the native H. themophilus TK-6 2-oxoglutarate carboxylase. The amino acids targeted for substitution were identified by comparing the H. themophilus TK-6 2-oxoglutarate carboxylase with its nearest mesophilic homolog from Candidatus Nitrospira defluvii.

The deletions, insertions and substitutions in the polypeptides described herein are not expected to produce radical changes in the characteristics of the polypeptide (e.g., the temperature at which the polypeptide is active). However, when it is difficult to predict the exact effect of the substitution, deletion or insertion in advance of doing so, one of skill in the art will appreciate that the effect can be evaluated by routine screening assays for the particular polypeptide activities of interest (e.g., succinyl CoA synthetase, 2-oxoglutarate:ferredoxin oxidoreductase, 2-oxoglutarate carboxylase, oxalosuccinate reductase SOR, aquaporin activity and the like) as described herein.

In some embodiments, the compositions of the invention can comprise active fragments of the polypeptide. As used herein, “fragment” means a portion of the reference polypeptide that retains the polypeptide activity of succinyl CoA synthetase, 2-oxoglutarate:ferredoxin oxidoreductase, 2-oxoglutarate carboxylase, oxalosuccinate reductase SOR, and/or aquaporin. A fragment also means a portion of a nucleic acid molecule encoding the reference polypeptide. An active fragment of the polypeptide can be prepared, for example, by isolating a portion of a polypeptide-encoding nucleic acid molecule that expresses the encoded fragment of the polypeptide (e.g., by recombinant expression in vitro), and assessing the activity of the fragment. Nucleic acid molecules encoding such fragments can be at least about 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, or 2000 contiguous nucleotides, or up to the number of nucleotides present in a full-length polypeptide-encoding nucleic acid molecule. As such, polypeptide fragments can be at least about 50, 60, 70, 80, 90, 100, 125, 150, 175, 200, 225, 250, 275, 300, 325, 350, 375, 400, 425, 450, 475, 500, 525, 550, 575, 600, 625, 650, 675, or 700 contiguous amino acid residues, or up to the total number of amino acid residues present in the full-length polypeptide.

Methods for assaying the activities of the crTCA cycle enzymes (e.g., succinyl CoA synthetase, 2-oxoglutarate:ferredoxin oxidoreductase, 2-oxoglutarate carboxylase, oxalosuccinate reductase and isocitrate lyase) are known in the art. Exemplary activity assays for the crTCA cycle enzymes are set forth below.

crTCA Cycle Reaction #1: Succinyl CoA Synthetase.

The succinyl CoA synthetase assay is a spectrophotometric method that measures the increase of absorbance at 232 nm in response to thioester formation. The standard reaction solution consists of 10 mM sodium succinate, 10 mM MgCl₂, 0.1 mM CoA, 0.1 mM DTT, 0.4 mM nucleotide (ATP or GTP) and 0.1 M KCl in 50 mM Tris-HCl (pH 7.4). The reaction is started with the addition of purified succinyl CoA synthetase or crude extract containing SCS. The reaction is monitored in a spectrophotometer set at 232 nm at 25° C. (See, e.g., Bailey et al. A dimeric form of Escherichia coli succinyl-CoA synthetase produced by site-directed mutagenesis. J. Mol. Biol. 285:1655-1666 (1999); Bridger et al. Succinyl coenzyme A synthetase from Escherichia coli. Methods Enzymol. 13:70-75 (1969))

For the LC/MS method of detection of succinyl CoA produced (LC-ESI-IT), the enzyme reactions are stopped by the addition of 30 μL of 15% (wt/vol) trifluoroacetic acid. A Nucleosil RP C18 (5 μm, 100-A pores; Knauer GmbH, Berlin, Germany) reverse-phased column serves to separate the CoA esters at 30° C. A 50 mM concentration of ammonium acetate (pH 5.0) adjusted with acetic acid (eluent A) and 100% (vol/vol) methanol (eluent B) serves as eluents. Elution occurs at a flow rate of 0.3 ml/min. Ramping is performed as follows: equilibration with 90% eluent A for 2 min before injection and 90 to 45% eluent A for 20 min, followed by holding for 2 min, and then a return to 90% eluent A within 5 min after injection. Detection of CoA esters occurs at 259 nm with a photodiode array detector. The instrument is tuned by direct infusion of a solution of 0.4 mM CoA at a flow rate of 10 μL/min into the ion source of the mass spectrometer to optimize the ESI-MS system for maximum generation of protonated molecular ions (parents) of CoA derivatives. The following tuning parameters are retained for optimum detection of CoA esters: capillary temperature, 300° C.; sheet gas flow, 12 liters/h; auxiliary gas flow, 6 liters/h; and sweep gas flow, 1 liter/h. The mass range is set to m/z 50 to 1,000 Da when running in the scan mode. The collision energy in the MS mode is set to 30 V. See, e.g., Schurmann et al. Novel Reaction of Succinyl Coenzyme A (Succinyl-CoA) Synthetase: Activation of 3-Sulfinopropionate to 3-Sulfinopropionyl-CoA in Advenella mimigardefordensis Strain DPN7T during Degradation of 3,3-Dithiodipropionic Acid. J. Bacteriol. 193(12):3078 (2011).

crTCA Cycle Reaction #2: 2-Oxoglutarate:Ferredoxin Oxidoreductase.

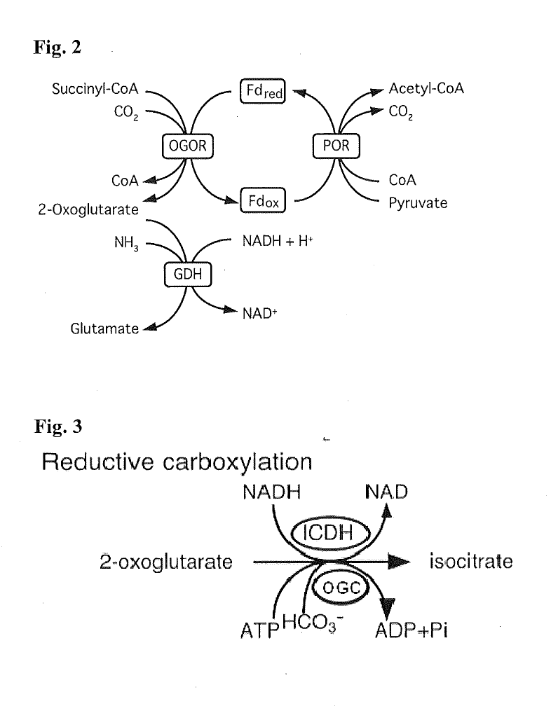

The assay for the forward reaction for 2-oxoglutarate:ferredoxin oxidoreductase (OGOR) is a coupled spectrophotometric assay based in the changes of NADH levels, which are measured at 340 nm. As shown in FIG. 2, the OGOR enzyme reaction is coupled with GDH catalyzed conversion of 2-oxoglutarate to glutamate, consuming NADH to NAD+. The pyruvate oxoreductase (POR) reaction reproduces reduced form of ferredoxin (Yamamoto et al. Carboxylation reaction catalyzed by 2-oxoglutarate:ferredoxin oxidoreductases from Hydrogenobacter thermophilus. Extremophiles. 14:79-85 (2010)).

For the reverse reaction for OGOR, enzymatic activity of recombinant OGOR in the cell-free extract is determined by 2-oxoglutarate dependent reduction of methyl viologen at 578 nm. The standard assay mixture contains 10 mM MOPS (pH 6.8), 1 mM MgCl₂, 1 mM DTT, 20 mM NaHCO3, 5 mM NH₄Cl, 0.25 mM CoA, 0.26 mM NADH, 100 mM pyruvate, 1 mM succinyl-CoA, and proteins (OGOR, POR, ferredoxin, and GDH). The gas phase in the quartz cell is replaced with argon. The reaction is initiated by addition of succinyl-CoA. The change in A340 (representing a decrease in the consumption of NADH) is measured using a spectrophotometer. The measurement is taken 30 s following succinyl-CoA addition. The reaction mixtures contain 50 mM Tris/HCl, pH 7.5, 5 mM sodium 2-oxoglutarate, 1 mM MgCl₂, 2.5 mM DTT, 0.1 mM CoA, 50 uM TPP, and 1 mM methyl viologen in a final volume of 2 ml. The reduction of methyl viologen is monitored at 578 nm. (See, e.g., Yun et al. Biochem. Biophys. Res. Comm. 282: 589-594 (2001); Wahl et al. J Biol Chem. 262: 10489-10496 (1987).

For the GC/MS method for the measurement of targeted metabolites including succinate, 2-oxoglutarate, glyoxylate, and citrate (GC-EI), the enzyme reactions are stopped by the addition of 30 μL of 15% (wt/vol) trifluoroacetic acid. GC/GC/MS experiments are performed using a LECO Pegasus III time-of-flight mass spectrometer with the 4D upgrade (LECO Corp., St. Joseph, Mich., USA). Column 1 is a 20 m Rtx-5 capillary column with an internal diameter of 250 μm and a film thickness of 0.5 μm and column 2 was a 2 m Rtx-200 (Restek, Bellefonte, Pa., USA) with a 180 μm internal diameter and 0.2 μm film thickness. The two columns are joined by a cryogenic modulator with a modulation period of 1.5 s with a hot pulse time of 0.40 s. Ultra high purity helium is used as the carrier gas at constant flow mode of 1 mL/min. 1 μL of a given sample is injected in triplicate in split-less mode via an Agilent 7683 autosampler. The inlet temperature is set at 280° C. The temperature program used for column 1 begins at 60° C. with a hold time of 0.25 min, then increased at 8° C./min to 280° C. with a hold time at 280° C. for 10 min. Column 2 is held in a separate oven which is initially set at 70° C. and followed the same temperature program as column 1. The ion source temperature is set to 250° C. Mass spectra are collected from m/z 40 to 600 at 100 spectra/s with a 5 min solvent delay (Yang et al. Journal of Chromatography A, 1216:3280-3289 (2009))

crTCA Cycle Reaction #3: 2-Oxoglutarate Carboxylase.

The assay for 2-oxoglutarate carboxylase is a spectrophotometric assay in which the reductive carboxylation of 2-oxoglutarate to isocitrate is monitored indirectly at 340 nm (measuring NADH oxidation). See FIG. 3 below. Note that this assay is actually measuring the combined reactions of crTCA Cycle Reaction #3 and #4 (OGC and oxalosuccinate reductase). The reaction mixture for this assay (total volume of 250 μl) is composed of 100 mM Bicine-KOH (prepared from 1 M stock solution of pH 8.5, adjusted at room temperature), 50 mM NaHCO₃, 10 mM 2-oxoglutarate, 10 mM Mg-ATP, 0.25 mM NADH, 3.6 mg of ICDH (from H. thermophilus, recombinant) and OGC. The reaction is started by the addition of NADH and OGC. NADH oxidation is monitored at 340 nm (e=6.3 mM-1 cm-1) for 1 min. One unit of activity is defined as 1 mmol of NADH oxidized per min (Aoshima et al. Mol. Microbiol. 62:748-759 (2006)). The GC/MS method for OGC is the same as that set forth for crTCA cycle reaction #2 above.

crTCA Cycle Reaction #4: Oxalosuccinate Reductase.

The assay provided herein for crTCA cycle reaction #3 (see, e.g., (Aoshima et al. Mol. Microbiol. 62:748-759 (2006)). For the LC/MS method for the detection of isocitrate produced (LC-ESI), chromatographic separation is carried out using a 250×4.6 mm (5 μm) Allure Organic Acids column (Restek Corp., Bellefonte, Pa.) fitted with a 10×4.6 mm (5 μm) guard column at 30° C. Mobile phase is water/methanol (85:15) containing 0.5% formic acid, delivered at 0.7 mL/min. The column effluent is split in a ratio of 1:1 before the ionization source. The injection volume is 10 μL. Two multiple reaction monitoring (MRM) transitions in the negative ion mode are used. The dwell time, interchannel delay, and interscan delay are 0.1, 0.02, and 0.1 s, respectively. Other operating parameters are as follows: capillary voltage, 3 kV; source and desolvation temperature, 120 and 350° C.; desolvation and cone gas flow rates, 900 and 50 L/h, respectively; cone voltage, 20 V; collision energy, 20 eV. (See, e.g., Ehling et al. J. Agric. Food Chem. 59:2229-2234 (2011)).

crTCA Cycle Reaction #5: Isocitrate Lyase.

This is a continuous spectrophotometric rate determination in which isocitrate lyase (ICL) converts isocitrate to succinate and glyoxylate. The glyoxylate is chemically converted to glyoxylate phenylhydrazone in the presence of phenylhydrazine. The glyoxylate phenylhydrazone is measured at 324 nm. The reaction mixture contains 30 mM imidazole (pH 6.8), 5 mM MgCl₂, 1 mM EDTA, 4 mM phenylhydrazine and 10 mM isocitrate. The reaction was performed at room temperature. After adding ICL, the reaction was continuously monitored at 324 nm (See, e.g., Chell et al. Biochemical Journal 173:165-177 (1978))

These assays can be performed on protein extracts from plants, plant parts (e.g., leaf, stem, seed, and the like) and plant cells (e.g., cell cultures comprising tissue culture, a suspension of plant cells such as algal cells, protoplasts and the like).

Incorporation of Glyoxylate into the Calvin Benson Cycle

The net product of the crTCA cycle is glyoxylate. To feed the assimilated carbon from glyoxylate into the Calvin Benson cycle, additional enzymes can be used to convert the glyoxylate into tartronic-semialdehyde (using glyoxylate carboligase) and then reduce the tartronic-semialdehyde into glycerate (using tartronic semialdehyde reductase). The resulting glycerate can then be phosphorylated by the chloroplastic glycerate kinase to glycerate phosphate, a Benson-Calvin intermediate. Thus, in addition to heterologous polynucleotides encoding polypeptides of the synthetic crTCA cycle as described herein, further embodiments of this invention comprise introducing into a plant, plant part and/or plant cell one or more heterologous polynucleotides encoding polypeptides that feed the products of the crTCA cycle of this invention into the Calvin Benson cycle (i.e., bridging enzymes).

By feeding the products (glyoxylate) of the synthetic crTCA cycle of this invention efficiently into the Calvin Benson cycle a further increase in carbon fixation and biomass production can be achieved in a plant, plant cell and/or plant part comprising the synthetic crTCA cycle polynucleotides. In some embodiments, heterologous polynucleotides encoding polypeptides that can feed the products of the synthetic crTCA cycle into the Calvin Benson cycle include, but are not limited to, a polynucleotide encoding a polypeptide having the enzyme activity of glyoxylate carboligase and/or a polynucleotide encoding a polypeptide having the enzyme activity of tartronic semialdehyde reductase. Thus, in some embodiments, the invention further provides introducing into a plant, plant part and/or plant cell one or more heterologous polynucleotides encoding polypeptides having the enzyme activity of glyoxylate carboligase and tartronic semialdehyde reductase to produce a stably transformed plant, plant part, and/or plant cell expressing said one or more heterologous polynucleotides to produce said polypeptides, thereby feeding the products of the synthetic crTCA cycle described herein into the Calvin Benson cycle and increasing carbon fixation and/or biomass production in said stably transformed plant, plant part and/or plant cell as compared to a control (e.g., a plant, plant part or plant cell that is not stably transformed with said one or more heterologous polynucleotides).

Accordingly, in some particular embodiments, a method for increasing carbon fixation and/or increasing biomass production in a plant is provided, comprising introducing into a plant, plant part and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase to produce a stably transformed plant, plant part, and/or plant cell expressing said one or more heterologous polynucleotides to produce said polypeptides, wherein the expression of the one or more heterologous polynucleotides encoding polypeptides having the enzyme activity of (a)-(g) results in the plant, plant part and/or plant cell having increased carbon fixation and/or increased biomass production as compared to a control (e.g., a plant, plant part and/or plant cell that is not stably transformed with said one or more heterologous polynucleotides encoding polypeptides having the enzyme activity of (a)-(g)). In some aspects, the method further comprises regenerating a stably transformed plant or plant part from the stably transformed plant cell, wherein expression of the one or more heterologous polynucleotides results in the stably transformed plant and/or plant part having increased carbon fixation and/or increased biomass production as compared to a control.

In representative embodiments of the invention, a heterologous polypeptide encoding a polypeptide having the enzyme activity of a glyoxylate carboligase can be the nucleotide sequence of SEQ ID NO:100, which encodes the amino acid sequence of SEQ ID NO:101 and heterologous polypeptide encoding a polypeptide having the enzyme activity of a tartronic semialdehyde reductase carboligase can be the nucleotide sequence of SEQ ID NO:102, which encodes the amino acid sequence of SEQ ID NO:103.

In additional embodiments, the activities of succinyl CoA synthetase, 2-oxoglutarate:ferredoxin oxidoreductase, 2-oxoglutarate carboxylase, oxalosuccinate reductase, isocitrate lyase, glyoxylate carboligase and/or tartronic semialdehyde reductase can be present in different polypeptides. In other embodiments, one or more of the enzyme activities can be present in a single polypeptide. Thus, for example, a single polypeptide can comprise the enzyme activity of at least two of the succinyl CoA synthetase, 2-oxoglutarate:ferredoxin oxidoreductase, 2-oxoglutarate carboxylase, oxalosuccinate reductase, isocitrate lyase, glyoxylate carboligase and/or tartronic semialdehyde reductase. In other embodiments, polypeptides having the enzyme activity of succinyl CoA synthetase, 2-oxoglutarate:ferredoxin oxidoreductase, 2-oxoglutarate carboxylase, oxalosuccinate reductase, isocitrate lyase, glyoxylate carboligase and/or tartronic semialdehyde reductase can be encoded by one or more polynucleotides. In still other embodiments, polypeptides having the enzyme activity of succinyl CoA synthetase, 2-oxoglutarate:ferredoxin oxidoreductase, 2-oxoglutarate carboxylase, oxalosuccinate reductase, isocitrate lyase, glyoxylate carboligase and/or tartronic semialdehyde reductase are each encoded by a different polynucleotide. When encoded by different polynucleotides, the different polynucleotides can be introduced in a single nucleic acid construct (e.g., expression cassette) or in two or more nucleic acid constructs (e.g., 2, 3, 4, 5, 6, 7, and the like).

### Superoxide Reductase

Reactive oxygen species (ROS) are generated in the cells of aerobic organisms during normal metabolic processes and have been identified to have an important role in cell signaling and homeostasis. However, high levels of ROS can be detrimental to an organism's cell structure and metabolism often resulting in cell death (i.e., oxidative stress). Most organisms have endogenous mechanisms for protecting them from potential damage by ROS, including enzymes such as superoxide dismutase, catalase and peroxide, and small antioxidant molecules. However, under conditions of abiotic stress, the levels of ROS can rise significantly making the endogenous protective mechanisms insufficient. By stably introducing a heterologous polynucleotide encoding SOR from an archaeon species into the cells of plants as described herein, said plants stably expressing the SOR have reduced reactive oxygen species and thereby increased tolerance to the environmental stresses that induce ROS production.

In other aspects, the invention further provides a method of reducing reactive oxygen species, reducing photorespiration, protecting the photosynthetic apparatus and/or surrounding membrane lipids, increasing photosynthetic efficiency, increasing tolerance to abiotic stress (e.g., heat, high light, drought, ozone, heavy metals, pesticides, herbicides, toxins, and/or anoxia), delaying senescence, reducing lignin polymerization, and increasing accessibility of cell wall cellulose in a plant, plant part and/or plant cell, comprising introducing into said plant, plant part and/or plant cell a heterologous polynucleotide encoding a superoxide reductase from an archaeon species to produce a stably transformed plant, plant part and/or plant cell expressing said heterologous polynucleotide encoding a superoxide reductase. In some embodiments, the delay of senescence resulting from the stably transformed plant expressing said heterologous polynucleotide encoding a superoxide reductase further results in said stably transformed plant having increased seed yield.

Accordingly, in some aspects, the present invention provides a method for increasing carbon fixation and/or increasing biomass production and reducing reactive oxygen species, protecting the photosynthetic apparatus and/or surrounding membrane lipids, reducing photorespiration, increasing photosynthetic efficiency, increasing tolerance to abiotic stress (e.g., heat, high light, drought, ozone, heavy metals, pesticides, herbicides, toxins, and/or anoxia), delaying senescence, reducing lignin polymerization and/or increasing accessibility of cell wall cellulose in a plant, plant part and/or plant cell to at least one enzyme, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, and a heterologous polynucleotide encoding a superoxide reductase from an archaeon species to produce a stably transformed plant, plant part and/or plant cell expressing said one or more heterologous polynucleotides encoding polypeptides having the enzyme activity of (a)-(e) and said heterologous polynucleotide encoding said superoxide reductase, wherein said stably transformed plant, plant part and/or plant cell has increased carbon fixation and/or increased biomass production, reduced photorespiration, reduced reactive oxygen species, protected photosynthetic apparatus and/or surrounding membrane lipids, increased photosynthetic efficiency, increased tolerance to abiotic stress (e.g., heat, high light, drought, ozone, heavy metals, pesticides, herbicides, toxins, and/or anoxia), delayed senescence, reduced lignin polymerization and/or increased accessibility of cell wall cellulose in said plant, plant part and/or plant cell to at least one enzyme as compared to a control (e.g., a plant, plant part, or plant cell not stably transformed with said one or more heterologous polynucleotides encoding polypeptides having the enzyme activity of (a)-(e) and said heterologous polynucleotide encoding said superoxide reductase). In some aspects, the method further comprises regenerating a stably transformed plant or plant part from said stably transformed plant cell, wherein said stably transformed plant and/or plant part expresses the one or more heterologous polynucleotides encoding polypeptides having the enzyme activity of the polypeptides of (a)-(e) above and the heterologous polynucleotide encoding said superoxide reductase, thereby increasing carbon fixation and/or increasing biomass production, reducing photorespiration, reducing reactive oxygen species, protecting photosynthetic apparatus and/or surrounding membrane lipids, increasing photosynthetic efficiency, increasing tolerance to abiotic stress (e.g., heat, high light, drought, ozone, heavy metals, pesticides, herbicides, toxins, and/or anoxia), delaying senescence, reducing lignin polymerization and/or increasing accessibility of cell wall cellulose to at least one enzyme in said plant and/or plant part as compared to a control.

In representative embodiments, the present invention provides a method for increasing carbon fixation and/or increasing biomass production and reducing or lowering reactive oxygen species, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (e) above and said heterologous polynucleotide encoding said superoxide reductase to produce said polypeptides (a) to (e) and said archaeon superoxide reductase, wherein said stably transformed plant, plant part and/or plant cell has increased carbon fixation and/or increased biomass production and reduced or lowered reactive oxygen species as compared to a control (e.g., a plant, plant part or plant cell not stably transformed with said one or more heterologous polynucleotides encoding polypeptides having the enzyme activity of (a)-(e) and said heterologous polynucleotide encoding said superoxide reductase).

In additional embodiments, the present invention provides a method for increasing carbon fixation and/or increasing biomass production in a plant and reducing or lowering reactive oxygen species, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (g) above and said heterologous polynucleotide encoding said superoxide reductase, wherein said stably transformed plant, plant part and/or plant cell has increased carbon fixation and/or increased biomass production and reduced/lowered reactive oxygen species as compared to a control (e.g., a plant, plant part or plant cell not stably transformed with said one or more heterologous polynucleotides encoding polypeptides having the enzyme activity of (a)-(g) and said heterologous polynucleotide encoding said superoxide reductase).

In additional embodiments, the present invention provides a method for increasing carbon fixation and/or increasing biomass production and protecting photosynthetic centers in a plant, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (e) above and said heterologous polynucleotide encoding said superoxide reductase to produce said polypeptides (a) to (e) and said archaeon superoxide reductase, wherein said stably transformed plant, plant part and/or plant cell has increased carbon fixation and/or increased biomass production and protected photosynthetic centers in a plant as compared to a control (e.g., a plant, plant part or plant cell not stably transformed with said one or more heterologous polynucleotides encoding polypeptides having the enzyme activity of (a)-(e) and said heterologous polynucleotide encoding said superoxide reductase).

In additional embodiments, the present invention provides a method for increasing carbon fixation and/or increasing biomass production in a plant and protecting photosynthetic centers in a plant, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (g) above and said heterologous polynucleotide encoding said superoxide reductase, wherein said stably transformed plant, plant part and/or plant cell has increased carbon fixation and/or increased biomass production and protected photosynthetic centers in a plant as compared to a control (e.g., a plant, plant part or plant cell not stably transformed with said one or more heterologous polynucleotides encoding polypeptides having the enzyme activity of (a)-(g) and said heterologous polynucleotide encoding said superoxide reductase).

In some embodiments, the present invention provides a method for increasing carbon fixation and/or increasing biomass production and delaying senescence in a plant, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (e) above and said heterologous polynucleotide encoding said superoxide reductase to produce said polypeptides (a) to (e) and said archaeon superoxide reductase, wherein said stably transformed plant, plant part and/or plant cell has increased carbon fixation and/or increased biomass production and delayed senescence in a plant as compared to a control (e.g., a plant, plant part or plant cell not stably transformed with said one or more heterologous polynucleotides encoding polypeptides having the enzyme activity of (a)-(e) and said heterologous polynucleotide encoding said superoxide reductase).

In other embodiments, the present invention provides a method for increasing carbon fixation and/or increasing biomass production in a plant and delaying senescence in a plant, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (g) above and said heterologous polynucleotide encoding said superoxide reductase, wherein said stably transformed plant, plant part and/or plant cell has increased carbon fixation and/or increased biomass production and delayed senescence in a plant as compared to a control (e.g., a plant, plant part or plant cell not stably transformed with said one or more heterologous polynucleotides encoding polypeptides having the enzyme activity of (a)-(g) and said heterologous polynucleotide encoding said superoxide reductase).

In additional embodiments, the present invention provides a method for increasing carbon fixation and/or increasing biomass production, protecting photosynthetic centers and delaying senescence in a plant, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (e) above and said heterologous polynucleotide encoding said superoxide reductase to produce said polypeptides (a) to (e) and said archaeon superoxide reductase, wherein said stably transformed plant, plant part and/or plant cell has increased carbon fixation and/or increased biomass production, protected photosynthetic centers and delayed senescence in a plant as compared to a control (e.g., a plant, plant part or plant cell not stably transformed with said one or more heterologous polynucleotides encoding polypeptides having the enzyme activity of (a)-(e) and said heterologous polynucleotide encoding said superoxide reductase).

In additional embodiments, the present invention provides a method for increasing carbon fixation and/or increasing biomass production, protecting photosynthetic centers and delaying senescence in a plant, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase and a heterologous polynucleotide encoding a superoxide reductase from an archaeon species to produce a stably transformed plant, plant part and/or plant cell expressing said one or more heterologous polynucleotides encoding polypeptides having the enzyme activity of (a) to (g) above and said heterologous polynucleotide encoding said superoxide reductase, wherein said stably transformed plant, plant part and/or plant cell has increased carbon fixation and/or increased biomass production, protected photosynthetic centers and delayed senescence in a plant as compared to a control (e.g., a plant, plant part or plant cell not stably transformed with said one or more heterologous polynucleotides encoding polypeptides having the enzyme activity of (a)-(g) and said heterologous polynucleotide encoding said superoxide reductase).

In some embodiments, the archaeon species can be a species from the genus Pyrococcus, a species from the genus Thermococcus, or a species from the genus Archaeoglobus. In other embodiments, the archaeon species can be Pyrococcus furiosus and the heterologous polynucleotide encoding a SOR can optionally comprise, consist essentially of, or consist of a nucleotide sequence of SEQ ID NO:72 or SEQ ID NO:73 and/or a nucleotide sequence having at least about 80% sequence identity to a nucleotide sequence of SEQ ID NO:72 or SEQ ID NO:73 (e.g., about 80%, about 85%, about 90%, about 95%, about 96%, about 97%, about 98%, about 99%, about 100% identity, and any range therein). In still other embodiments, an amino acid sequence of superoxide reductase can optionally comprise, consist essentially of, or consist of the amino acid sequence of SEQ ID NO:74 or SEQ ID NO:75 and/or an amino acid sequence having at least about 80% sequence identity to the amino acid sequence of SEQ ID NO:74 or SEQ ID NO:75 (e.g., about 80%, about 85%, about 90%, about 95%, about 96%, about 97%, about 98%, about 99%, about 100% identity, and any range therein).

Methods for detecting and quantifying ROS or oxidized cell components are well known in the art and include, but are not limited to: the nitroblue tetrazolium assay (Fryer et al. J Exp Bot 53: 1249-1254 (2002); Fryer et al. Plant J 33: 691-705 (2003)) and acridan lumigen PS-3 assay (Uy et al. Journal of Biomolecular Techniques 22:95-107 (2011) for detection of superoxide; the ferrous ammonium sulfate/xylenol orange (FOX) method (Wolff, Methods Enzymol 233: 182-189 (1994); Im et al. Plant Physiol 151:893-904 (2009)) for detection of peroxide; the thiobarbituric acid assay (TBA) (Draper and Hadley, Methods Enzymol 186:421-431 (1990); Hodges et al. Planta 207: 604-611 (1999)) and the mass spectrometric determination of peroxidated lipids (Deighton et al. Free Radic Res 27: 255-265 (1997)) for detection of lipid peroxidation; the assay for 8-hydroxy-2′-deoxygunanosine in DNA (Bialkowski and Olinski, Acta Biochim Pol 46: 43-49 (1999)) for the detection of nucleic acid oxidation; and the reaction of oxidized protein with 2,4-dinitrophenylhydrazine (DPNH) (Levine et al. Methods Enzymol 233:346-357 (1994)) for detection of protein oxidation.

A “photosynthetic apparatus and surrounding membrane lipids” is a complex of specific proteins, pigments, lipids and other co-factors that includes the two photosystems and the proteins involved in electron and proton transfer between them as well as the ATPase that function in the primary energy conversion reactions of photosynthesis. During the process of photosynthesis electron transfer reactions are promoted along a series of protein-bound co-factors and it is these electron transfer steps that are the initial phase of a series of energy conversion reactions, ultimately resulting in the production of chemical energy during photosynthesis. Notably, reactive oxygen species can be generated during photosynthetic electron transfer resulting in oxidative damage to the photosynthetic reaction centers. Thus, the present invention protects the photosynthetic apparatus and surrounding membrane lipids by reducing the reactive oxygen species generated during photosynthetic electron transfer.

Methods for measuring “photosynthetic efficiency” or “photosynthesis rate” and thus measuring the protection of photosynthetic apparatus and/or its surrounding membrane lipids are known in the art and include, for example, fluorescence and gas exchange (CO₂, O₂, H₂O) measurements (e.g. Licor), analyzing the chlorophyll content and composition using light spectroscopy, and comparing protein content and turnover of photocenters (Chow et al. Photosynthesis Research: 1-12 (2012) and Hideg et al. Plant and Cell Physiology 49: 1879-1886 (2008)).

Methods for measuring photorespiration are known in the art. Thus, photorespiration can be indirectly measured by changes in the CO₂-saturation curve using fluorescence and gas exchange measurements (e.g., LiCOR) or via ¹⁸O₂ incorporation. Alternatively, determining the ratio of serine to glycine in actively photosynthesizing leaves can be used to measure photorespiration. Other ways that changes in photorespiration can be shown include comparing biomass productivity or photosynthesis under different CO₂:O₂ environments. See, e.g., Hideg et al. Plant and Cell Physiology 49: 1879-1886 (2008); and Berry et al. Plant Physiol 62:954-967 (1978).

Photosynthetic efficiency is the fraction of light energy converted into chemical energy during photosynthesis. Saturating pulse fluorescence measurements can be used to measure photosynthetic efficiency. CO₂ and O₂ exchange methods can also be used. A number of plant and algae studies have been done, which demonstrate that photosynthetic efficiency decreases when plants are exposed to ROS (Ganesh et al. Biotechnol Bioeng 96(6):1191-8 (2007); Zhang and Xing. Plant Cell Physiology 49(7):1092-1111 (2008)).

“Abiotic stress” or “environmental stress” as used herein means any outside, nonliving, physical or chemical factors or conditions that induce ROS production. Thus, in some embodiments of the invention, an abiotic or environmental stress can include, but is not limited to, high heat, high light, drought, ozone, heavy metals, pesticides, herbicides, toxins, and/or anoxia (i.e., root flooding). In some embodiments, environmental/abiotic stress for organisms used in fermentation can include but is not limited to, high metabolic flux and/or high fermentation product accumulation.

Parameters for the abiotic stress factors are species specific and even variety specific and therefore vary widely according to the species/variety exposed to the abiotic stress. Thus, for example, while one species may be severely impacted by a high temperature of 23° C., another species may not be impacted until at least 30° C., and the like. Temperatures above 30° C. result in, for example, dramatic reductions in the yields of many plant crops including algae. This is due to reductions in photosynthesis that begin at approximately 20-25° C., and the increased carbohydrate demands of crops growing at higher temperatures. The critical temperatures are not absolute, but vary depending upon such factors as the acclimatization of the organism to prevailing environmental conditions. In addition, because organisms are often exposed to multiple abiotic stresses at one time, the interaction between the stresses affects the response. For example, damage to a plant from excess light occurs at lower light intensities as temperatures increase beyond the photosynthetic optimum. Water stressed plants are less able to cool overheated tissues due to reduced transpiration, further exacerbating the impact of excess (high) heat and/or excess (high) light intensity. Thus, the particular parameters for high/low temperature, light intensity, drought and the like, which can negatively impact an organism will vary with species, variety, degree of acclimatization and the exposure to a combination of environmental conditions.

Methods for measuring reduced lignin polymerization are known in the art. Such methods include, but are not limited to, histochemical staining (Nakano et al. The Detection of Lignin Methods in Lignin Chemistry. Berlin: Springer-Verlag (1992)). Lignin content can also be determined using the Klason procedure (Dence et al. Lignin Determination. Berlin: Springer-Verlag (1992)). In addition, NMR (Kim et al. Bio. Res. 1:56-66 (2008)) or thioacidolysis procedure (Lapierre et al. Res. Chem. Intermed. 21:397-412 (1995)) followed by GC-MS or LC-MS can be used for quantification of lignin monomers.

Lignin polymerization occurs through the radical coupling of hydroxycinnamyl subunits (i.e., monolignols, e.g., coniferyl (CA), sinapyl (SA), and p-coumaryl alcohols (p-CA)). Monolignols require ROS for polymerization (Boerjan et al. Annu. Rev. Plant Biol. 54:519-546 (2003)). Lignin polymers are deposited predominantly in the walls of secondarily thickened cells, making them rigid and impervious. Further, the presence of the lignin polymers in the cell wall reduces the accessibility of the cell wall polysaccharides (cellulose and hemicellulose) to microbes and microbial degradation. As a consequence of its ability to protect the cellulose and hemicellulose in the cell wall from microbial degradation, the presence of lignin is also a limiting factor in the process of converting of plant biomass to biofuels. However, in representative embodiments, the present invention provides methods of reducing lignin polymerization by stably introducing into the cell wall of a plant or plant part, a heterologous polynucleotide encoding a SOR from an archaeon species, thereby reducing the ROS and reducing lignin polymerization in said plant, plant part and/or plant cell. Further, a reduction in lignin polymerization in a plant, plant part and/or plant cell provides the enzymes used in biofuel production greater accessibility to the cellulose and hemicellulose.

### CO2 Transporter

In further aspects of the invention, a method for increasing CO₂ uptake into a plant, plant part and/or plant cell is provided by expression of high affinity CO₂ transporters in a plant, plant part and/or plant cell. Slow diffusion of CO₂ across cell wall and inner chloroplast membrane limits photosynthetic rates. A high affinity CO₂ transporter such as an aquaporin with high similarity to the human CO₂ pore (AQP1) has been identified in tobacco (NtAQP1) and shown to facilitate CO₂ membrane transport in plants (Uehlein et al. Nature 425(6959): 734-7 (2003); Uehlein et al. Plant Cell 20(3):648-57 (2008); Flexas et al. Plant J. 48(3):427-39 (2006)). NtAQP1 is localized to the inner chloroplast envelope membrane as well as to mesophyll cell plasma membranes (Uehlein et al. Plant Cell 20(3):648-57 (2008)). Overexpression of NtAQP1 in tobacco increased net photosynthesis at ambient CO₂ levels to 136%, and led to doubling of leaf growth rate.

Therefore, in some embodiments, the present invention uses native and modified high-affinity CO₂/bicarbonate specific transporters from marine eukaryotes as well as from prokaryotic extremophiles (archaea and bacteria) (e.g. from the marine microalgae Dunaliella spp.; and/or Hydrogenobacter thermophilis). These transporters can function under high temperature, alkaline conditions and in aquatic environments where the ambient CO₂ concentration is very low. Expression of these high affinity/extremophile CO₂/biocarbonate transporters in plants (including algae) may overcome limitations in CO₂/biocarbonate conductivity in the plasma membrane and chloroplast membrane for efficient and effective CO₂/biocarbonate assimilation into biomass. Specifically, CO₂/biocarbonate transporters from high pH tolerant and high temperature tolerant extremophiles may enable specificity and uptake rates under conditions that favor CO₂ loss from aqueous environments.

Accordingly, in additional embodiments of the invention, a method of increasing CO₂ uptake into a plant, plant part and/or plant cell is provided, comprising introducing into a plant, plant part, and/or plant cell a heterologous polynucleotide encoding an aquaporin to produce a stably transformed plant, plant part, and/or plant cell expressing said heterologous polynucleotide to produce said aquaporin, thereby increasing CO₂ uptake into said stably transformed plant, plant part and/or plant cell as compared to a plant, plant part and/or plant cell not stably transformed with said aquaporin. In some embodiment, the aquaporin is from a plant (including, but not limited to, a saltwater algae), an extremophile archea and/or extremophile bacteria.

In further aspects, the present invention provides a method for increasing carbon fixation and/or increasing biomass production and increasing CO₂ uptake in a plant, plant part and/or plant cell, the method comprising introducing into a plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, and a heterologous polynucleotide encoding an aquaporin to produce a stably transformed plant, plant part and/or plant cell expressing said one or more heterologous polynucleotides encoding polypeptides having the enzyme activity of (a)-(e) and said heterologous polynucleotide encoding said aquaporin, wherein said stably transformed plant, plant part and/or plant cell has increased carbon fixation and/or increased biomass production and increased CO₂ uptake as compared to a control (e.g., a plant, plant part, or plant cell not stably transformed with said one or more heterologous polynucleotides encoding polypeptides having the enzyme activity of (a)-(e) and said heterologous polynucleotide encoding aquaporin). In some aspects, the method further comprises regenerating a stably transformed plant or plant part from said stably transformed plant cell, wherein the stably transformed plant and/or plant part has increased carbon fixation and/or increased biomass production, and increased CO₂ uptake as compared to a control.

In some embodiments, the heterologous polynucleotide encoding said aquaporin is constitutively expressed, thereby overriding any endogenous developmental and/or tissue specific aquaporin expression in the plant, plant part and/or plant cell (See, e.g., Lian et al., Plant Cell Physiol 45: 481-489 (2004), Sade et al., New Phytol 181: 651-661 (2009), Sade et al., Plant Phys. 152:245-254 (2010)).

In additional embodiments, the present invention provides a method for increasing carbon fixation and/or increasing biomass production in a plant and increasing CO₂ uptake, the method comprising: introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase and a heterologous polynucleotide encoding an aquaporin to produce a stably transformed plant, plant part and/or plant cell expressing said one or more heterologous polynucleotides encoding polypeptides having the enzyme activity of (a) to (g) above and said heterologous polynucleotide encoding said aquaporin, wherein the stably transformed plant, plant part and/or plant cell has increased carbon fixation and/or increased biomass production and increased CO₂ uptake as compared to a control (e.g., a plant, plant part, or plant cell not stably transformed with said one or more heterologous polynucleotides encoding polypeptides having the enzyme activity of (a)-(g) and said heterologous polynucleotide encoding said aquaporin).

In further embodiments, the invention provides a method for increasing carbon fixation and/or increasing biomass production, reducing reactive oxygen species, protecting photosynthetic centers, delaying senescence, increasing abiotic stress tolerance (e.g., drought tolerance) and increasing CO₂ uptake in a plant, comprising introducing into a plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, a heterologous polynucleotide encoding a superoxide reductase from an archaeon species and a heterologous polynucleotide encoding an aquaporin to produce a stably transformed plant, plant part and/or plant cell expressing said one or more heterologous polynucleotides encoding polypeptides having the enzyme activity of (a)-(e), said heterologous polynucleotide encoding archaeon superoxide reductase, and said heterologous polynucleotide encoding aquaporin, wherein the stably transformed plant, plant part and/or plant cell has increased carbon fixation and/or increased biomass production, reduced reactive oxygen species, delayed senescence, increased abiotic stress tolerance (e.g., drought tolerance) and protected photosynthetic centers and expression of said heterologous polynucleotide encoding said aquaporin results in the plant, plant part and/or plant cell having increased CO₂ uptake as compared to a control (e.g., a plant, plant part, or plant cell not stably transformed with said one or more heterologous polynucleotides encoding polypeptides having the enzyme activity of (a)-(e), said heterologous polynucleotide encoding archaeon superoxide reductase and said heterologous polynucleotide encoding aquaporin). In some aspects, the method further comprises regenerating a stably transformed plant and/or plant part from said stably transformed plant cell, wherein said stably transformed plant and/or plant part has increased carbon fixation and/or increased biomass production, reduced reactive oxygen species, delayed senescence, increased abiotic stress tolerance, protected photosynthetic centers and increased CO₂ uptake as compared to control.

In additional embodiments, the present invention provides a method for increasing carbon fixation and/or increasing biomass production in a plant, reducing reactive oxygen species, protecting photosynthetic centers, delaying senescence, increasing abiotic stress tolerance and increasing CO₂ uptake, the method comprising introducing into said plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase, a heterologous polynucleotide encoding a superoxide reductase from an archaeon species and a heterologous polynucleotide encoding an aquaporin to produce a stably transformed plant, plant part and/or plant cell expressing said one or more heterologous polynucleotides encoding polypeptides having the enzyme activity of (a)-(g), said heterologous polynucleotide encoding archaeon superoxide reductase and said heterologous polynucleotide encoding aquaporin, wherein the stably transformed plant, plant part and/or plant cell has increased carbon fixation and/or increased biomass production, reduced reactive oxygen species, protected photosynthetic centers, delayed senescence, increased abiotic stress tolerance, and increased CO₂ uptake as compared to a control (e.g., a plant, plant part, or plant cell not stably transformed with said one or more heterologous polynucleotides encoding polypeptides having the enzyme activity of (a)-(g), said heterologous polynucleotide encoding archaeon superoxide reductase and said heterologous polynucleotide encoding aquaporin). In some aspects, the method further comprises regenerating a stably transformed plant or plant part from said stably transformed plant cell, wherein said stably transformed plant and/or plant part has increased carbon fixation and/or increased biomass production, reduced reactive oxygen species, protected photosynthetic centers, delayed senescence, increased abiotic stress tolerance, and increased CO₂ uptake as compared to a control.

In representative embodiments, a heterologous polynucleotide encoding an aquaporin can optionally comprise, consist essentially of or consist of a nucleotide sequence of SEQ ID NO:76, SEQ ID NO:78 and/or SEQ ID NO:80, or a nucleotide sequence having substantial identity to said nucleotide sequences of SEQ ID NO:76, SEQ ID NO:78 and/or SEQ ID NO:80. In other embodiments, an amino acid sequence of an aquaporin can optionally comprise, consist essentially of or consist of the amino acid sequence of SEQ ID NO:77, SEQ ID NO:79 and/or SEQ ID NO:81, or an amino acid sequence having substantial identity to said nucleotide sequences of the amino acid sequence of SEQ ID NO:77, SEQ ID NO:79 and/or SEQ ID NO:81.

Inhibitor of Cell Wall Invertase Inhibitor (cwII)

In further aspects of the invention, a method for increasing sucrose partitioning into fruits and/or seeds of a plant is provided, the method comprising expressing in a plant an inhibitor of cell wall invertase inhibitor (cwII). The export of sugars occurs from photosynthesizing mesophyll cells through the cell wall into the phloem/companion cell complex which carries sugars via mass flow to non-photosynthetic tissues. Phloem unloading occurs either via the cell wall (apoplasm) or via plasmodesmata (Koch, K. Curr Opin Plant Biol. 7(3):235-46 (2004); Ward et al. Intl. Rev. Cytol. —a Survey of Cell Biol. 178:41-71 (1998)). Export and import through the apoplasm are controlled by the activity of cell wall invertase (cwI), which hydrolyzes sucrose into glucose and fructose and is regulated by a specific inhibitor protein (cwII) (Ruan et al. Molecular Plant. 3(6):942-955 (2010); Greiner et al. Plant Physiol. 116(2):733-42 (1998)). Two general approaches have been used to modify sucrose flux: overexpression of cwI or repression of its inhibitor protein cwII (Wang et al. Nature Genetics. 40(11):1370-1374 (2008); Sonnewald et al. Plant J. 1(1):95-106 (1991); von Schaewen et al. Embo J. 9(10):3033-44 (1990); Zanor, M. I., et al. Plant Physiology 150(3):1204-1218 (2009); Jin et al. Plant Cell. 21(7):2072-89 (2009); Greiner et al. Nat Biotechnol. 17(7):708-11 (1999)).

In general, low cwI activity is thought to increase sucrose export from the source tissue, and high cwI activity increases sucrose unloading into fruits and seeds/grains. Quantitative trait loci analysis for fruit size in tomato (Lin5), and grain size in rice (GIF1) and maize (MN1) identified mutations in cell-wall invertases that led to reduction in its activity in pedicel/fruit tissues (Wang et al. Nature Genetics. 40(11):1370-1374 (2008);

Fridman et al. Science 305(5691):1786-1789 (2004); Cheng et al. Plant Cell. 8(6):971-983 (1996)) as key regulators for phloem unloading and therefore determinants of seed and fruit size. Fruit-specific suppression of the cell wall invertase inhibitor (CwII) in tomato and rice led to increases in net seed/grain weight of 22% and 10%, respectively (Wang et al. Nature Genetics. 40(11):1370-1374 (2008); Jin et al. Plant Cell. 21(7):2072-89 (2009)). Accordingly, the present invention further provides methods to direct assimilate partitioning into fruit/seeds by suppressing cwII in plants using, for example, RNAi technology, thereby increasing assimilate partitioning into fruits and/or seeds of said plants.

Cell wall invertase inhibitors (cwII) are small peptides, with molecular masses (Mr) ranging from 15 to 23 kD, and may be localized to either the cell wall or vacuole (Krausgrill et al., Plant Journal 13(2): 275-280 (1998); Greiner et al. Plant Physiol. 116(2):733-42 (1998) Greiner et al. Australian Journal of Plant Physiology 27(9): 807-814 (2000). The functionality of these inhibitors has been determined largely by in vitro assays of their recombinant proteins (e.g., Greiner et al. Plant Physiol. 116(2):733-42 (1998); Bate et al., Plant Physiology 134 (1): 246-254 (2004). Cell wall and vacuolar invertases are highly stable proteins due to the presence of glycans, and as a result their activity may be highly dependent on posttranslational regulation by its inhibitory protein (Greiner et al., Australian Journal of Plant Physiology 27(9): 807-814 2000; Hothorn et al., Plant Cell 16 (12): 3437-3447 (2004); Rausch and Greiner, Biochim Biophys Acta 1696(2):253-61 (2004)). Sequence comparisons with the known invertase inhibitors (Hothorn et al. Proc Natl Acad Sci USA. 107(40):17427-32 (2010)).

Methods for developing antisense silencing constructs or inhibitors generally are well known in the art. Thus, for example, for the purpose of silencing an inhibitor of cell wall invertase (cwII) of interest, the nucleotide sequence of the cwII of interest can be identified by sequence homology to known cwIIs using techniques that are standard in the art (See, e.g., Jin et al. Plant Cell 21:2072-2089 (2009)). Based on the nucleotide sequence of the cwII of interest, antisense nucleotide sequences can be prepared. Thus, for example, a cwII from Camelina sativa can be used to prepare RNAi for inhibition of such cwII. Accordingly, in some embodiments of the invention a method of directing assimilate partitioning into fruits and/or seeds of a plant is provided, comprising introducing into a plant cell a heterologous polynucleotide encoding an inhibitor of cell wall invertase inhibitor (cwII); regenerating a plant from said plant cell comprising said heterologous polynucleotide encoding said inhibitor to produce a stably transformed plant expressing said heterologous polynucleotide to produce said inhibitor of cell wall invertase inhibitor, thereby directing assimilate partitioning into fruits and/or seeds of said stably transformed plant as compared to a control (e.g., a plant not stably transformed with said inhibitor of CwII). In some embodiments, the inhibitor of cwII can be a RNAi. An exemplary RNAi inhibitor of cwII can be a sequence-specific inverted repeat (sense intron-antisense). In representative embodiments, an RNAi useful with this invention for inhibition of cwII can be the nucleotide sequences of SEQ ID NOs:106-108, or any fragment thereof capable of inhibiting cwII. In particular embodiments, endogenous camelina promoters of the cell wall invertase inhibitors (e.g., SEQ ID NO:104, SEQ ID NO:105) can be used in fusion with cwII RNAi to repress the transcript abundance of cell wall invertase inhibitors.

In further embodiments, the present invention provides a method for increasing carbon fixation and/or increasing biomass production and directing assimilate partitioning into fruits and/or seeds in a plant, the method comprising introducing into a plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, and a heterologous polynucleotide encoding an inhibitor of cell wall invertase inhibitor (cwII) to produce a stably transformed plant cell expressing said one or more heterologous polynucleotides encoding polypeptides having the enzyme activity of (a)-(e) and said heterologous polynucleotide encoding said inhibitor of cwII; and regenerating a stably transformed plant from said stably transformed plant cell, wherein the stably transformed plant has increased carbon fixation and/or increased biomass production and increased assimilate partitioning into fruits and seeds of said stably transformed plant as compared to a control (e.g., a plant not stably transformed with said one or more heterologous polynucleotides encoding polypeptides having the enzyme activity of (a)-(e) and said heterologous polynucleotide encoding said inhibitor of cwII).

In still further embodiments, the present invention provides a method for increasing carbon fixation and/or increasing biomass production and directing assimilate partitioning into fruits and/or seeds of a plant, the method comprising: introducing into a plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase and a heterologous polynucleotide encoding an inhibitor of cell wall invertase inhibitor (cwII) to produce a stably transformed plant cell expressing said one or more heterologous polynucleotides encoding polypeptides having the enzyme activity of (a) to (g) above and said heterologous polynucleotide encoding said inhibitor of cwII; and regenerating a stably transformed plant from said stably transformed plant cell, wherein said stably transformed plant has increased carbon fixation and/or increased biomass production and increased assimilate partitioning into fruits and seeds of said stably transformed plant as compared to a control (e.g., a plant not stably transformed with said one or more heterologous polynucleotides encoding polypeptides having the enzyme activity of (a)-(g) and said heterologous polynucleotide encoding said inhibitor of cwII).

In further embodiments, the invention provides a method for increasing carbon fixation and/or increasing biomass production, reducing reactive oxygen, protecting photosynthetic centers, delaying senescence (thereby, for example, increasing seed yield) and directing assimilate partitioning into fruits and/or seeds in a plant, comprising introducing into a plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, a heterologous polynucleotide encoding a superoxide reductase from an archaeon species and a heterologous polynucleotide encoding an inhibitor of cell wall invertase inhibitor (cwII) to produce a stably transformed plant cell expressing said one or more heterologous polynucleotides encoding polypeptides having the enzyme activity of (a)-(e), said heterologous polynucleotide encoding archaeon superoxide reductase, and said heterologous polynucleotide encoding the inhibitor of cwII; and regenerating a stably transformed plant from said stably transformed plant cell, wherein said stably transformed plant has increased carbon fixation and/or increased biomass production, reduced reactive oxygen species, protected photosynthetic centers, delayed senescence and increased assimilate partitioning into fruits and seeds of said stably transformed plant as compared to a control (e.g., a plant not stably transformed with said one or more heterologous polynucleotides encoding polypeptides having the enzyme activity of (a)-(e), said heterologous polynucleotide encoding a superoxide reductase and said heterologous polynucleotide encoding said inhibitor of cwII). In some embodiments, the one or more heterologous polynucleotides encoding polypeptides having the enzyme activity of (a) to (e) further comprises polypeptides having the enzyme activity of (f) glyoxylate carboligase and (g) tartronic semialdehyde reductase.

In additional embodiments, the present invention provides a method for increasing carbon fixation and/or increasing biomass production in a plant, reducing reactive oxygen species, protecting photosynthetic centers, delaying senescence, increasing CO₂ uptake and/or increasing assimilate partitioning into fruits and/or seeds in a plant, the method comprising: introducing into a plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, (g) tartronic semialdehyde reductase, a heterologous polynucleotide encoding a superoxide reductase from an archaeon species, a heterologous polynucleotide encoding an aquaporin and a heterologous polynucleotide encoding an inhibitor of cell wall invertase inhibitor (cwII) to produce a stably transformed plant cell expressing said one or more heterologous polynucleotides encoding polypeptides having the enzyme activity of (a)-(g), said heterologous polynucleotide encoding archaeon superoxide reductase, said heterologous polynucleotide encoding aquaporin and said heterologous polynucleotide encoding an inhibitor of cell wall invertase inhibitor (cwII); regenerating a stably transformed plant from said stably transformed plant cell, wherein the stably transformed plant has increased carbon fixation and/or increased biomass production, reduced reactive oxygen species, protected photosynthetic centers, delayed senescence, increased CO₂ uptake and increased assimilate partitioning into fruits and seeds of said stably transformed plant as compared to a control (e.g., a plant not stably transformed with said one or more heterologous polynucleotides encoding polypeptides having the enzyme activity of (a)-(g), said heterologous polynucleotide encoding superoxide reductase from an archaeon species, said heterologous polynucleotide encoding aquaporin and said heterologous polynucleotide encoding the inhibitor of cwII).

### Expression Cassettes

In some embodiments, the heterologous polynucleotide encoding polypeptides having the enzyme activity of succinyl CoA synthetase, 2-oxoglutarate:ferredoxin oxidoreductase, 2-oxoglutarate carboxylase, oxalosuccinate reductase and isocitrate lyase (e.g., the polynucleotides encoding the crTCA cycle polypeptides) as well as any other heterologous polynucleotide encoding a polypeptide or functional nucleic acid of interest (e.g., a heterologous polynucleotide encoding a polypeptide having activity of a glyoxylate carboligase, a tartronic semialdehyde reductase, a heterologous polynucleotide encoding a superoxide reductase from an archaeon species, a heterologous polynucleotide encoding an aquaporin, and/or a heterologous polynucleotide encoding an inhibitor of cell wall invertase inhibitor) can be comprised within an expression cassette. As used herein, “expression cassette” means a recombinant nucleic acid molecule comprising at least one polynucleotide sequence of interest (e.g., a heterologous polynucleotide encoding a synthetic crTCA cycle polypeptide, an aquaporin, an SOR, an inhibitor of cwII, and the like), wherein said recombinant nucleic acid molecule is operably associated with at least a control sequence (e.g., a promoter). Thus, some embodiments of the invention provide expression cassettes designed to express a recombinant nucleic acid molecule/heterologous polynucleotide encoding polypeptides having the enzyme activity of succinyl CoA synthetase, 2-oxoglutarate:ferredoxin oxidoreductase, 2-oxoglutarate carboxylase, oxalosuccinate reductase, isocitrate lyase, glyoxylate carboligase, tartronic semialdehyde reductase, a heterologous polynucleotide encoding superoxide reductase from an archaeon species, a heterologous polynucleotide encoding an aquaporin and/or a heterologous polynucleotide encoding an inhibitor of cwII.

An expression cassette comprising a recombinant nucleic acid molecule may be chimeric, meaning that at least one of its components is heterologous with respect to at least one of its other components. An expression cassette may also be one that is naturally occurring but has been obtained in a recombinant form useful for heterologous expression.

In some embodiments, the heterologous polynucleotides encoding the polypeptides having the enzyme activity of succinyl CoA synthetase, 2-oxoglutarate:ferredoxin oxidoreductase, 2-oxoglutarate carboxylase, oxalosuccinate reductase and isocitrate lyase can be comprised in a single expression cassette. The expression cassette can be operably linked to a promoter that drives expression of all of the polynucleotides comprised in the expression cassette and/or the expression cassette can comprise one or more promoters operably linked to one or more of the heterologous polynucleotides for driving the expression of said heterologous polynucleotides. In other embodiments, the heterologous polynucleotides encoding the polypeptides having the enzyme activity of succinyl CoA synthetase, 2-oxoglutarate:ferredoxin oxidoreductase, 2-oxoglutarate carboxylase, oxalosuccinate reductase and/or isocitrate lyase can be comprised in more than one expression cassette.

When the heterologous polynucleotides are comprised within more than one expression cassette, said heterologous polynucleotides encoding the polypeptides for the crTCA cycle of this invention can be introduced into plants singly or more than one at a time using co-transformation methods as known in the art. In addition to transformation technology, traditional breeding methods as known in the art can be used to assist in introducing into a single plant each of the polynucleotides encoding the polypeptides of the crTCA cycle as described herein and/or any other polynucleotides of interest in addition to those of the crTCA cycle as described herein (e.g., polynucleotides encoding a superoxide reductase, polynucleotides encoding an aquaporin polypeptide, polynucleotides encoding glyoxylate carboligase, tartronic semialdehyde reductase and/or an inhibitor of cell wall invertase inhibitor as described herein) to produce a plant, plant part, and/or plant cell comprising and expressing each of the heterologous polynucleotides of interest.

Any promoter useful for initiation of transcription in a cell of a plant can be used in the expression cassettes of the present invention. A “promoter,” as used herein, is a nucleotide sequence that controls or regulates the transcription of a nucleotide sequence (i.e., a coding sequence) that is operably associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” A promoter can be identified in and isolated from the organism to be transformed and then inserted into the nucleic acid construct to be used in transformation of the organism.

The choice of promoter will vary depending on the temporal and spatial requirements for expression, and also depending on the host cell to be transformed. Thus, for example, expression of the heterologous polynucleotide encoding the polypeptides of the crTCA cycle as described herein can be in any plant, plant part, (e.g., in leaves, in stalks or stems, in ears, in inflorescences (e.g. spikes, panicles, cobs, etc.), in roots, seeds and/or seedlings, and the like), or plant cells (including algae cells). For example, in the case of a multicellular organism such as a plant where expression in a specific tissue or organ is desired, a tissue-specific or tissue preferred promoter can be used (e.g., a root specific/preferred promoter). In contrast, where expression in response to a stimulus is desired a promoter inducible by stimuli or chemicals can be used. Where continuous expression at a relatively constant level is desired throughout the cells or tissues of an organism a constitutive promoter can be chosen.

Thus, promoters useful with the invention include, but are not limited to, those that drive expression of a nucleotide sequence constitutively, those that drive expression when induced, and those that drive expression in a tissue- or developmentally-specific manner. These various types of promoters are known in the art. Promoters can be identified in and isolated from the plant to be transformed and then inserted into the expression cassette to be used in transformation of the plant.

Non-limiting examples of a promoter include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al., (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al., (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al., (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-I,5-bisphosphate carboxylase” pp. 29-39 In: Genetic Engineering of Plants (Hollaender ed., Plenum Press 1983; and Poulsen et al., (1986) Mol. Gen. Genet. 205:193-200), Ti plasmid mannopine synthase promoter (Langridge et al., (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al., (1987) Mol. Gen. Genet. 207:90-98; Langridge et al., (1983) Cell 34:1015-1022; Reina et al., (1990) Nucleic Acids Res. 18:6425; Reina et al. (1990) Nucleic Acids Res. 18:7449; and Wandelt et al., (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J 10:2605-2612).

Particularly useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in an organism through the application of an exogenous chemical regulator. Regulation of the expression of nucleotide sequences of the invention via promoters that are chemically regulated enables the polypeptides of the invention to be synthesized only when, for example, a crop of plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters useful with plants are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid (e.g., the PR1a system), steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al., (1988) Genetics 119:185-197), the MPI proteinase inhibitor promoter (Cordero et al., (1994) Plant J. 6:141-150), and the glyceraldehyde-3-phosphate dehydrogenase promoter (Kohler et al. (1995) Plant Mol. Biol. 29:1293-1298; Martinez et al., (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

In some particular embodiments, promoters useful with algae include, but are not limited to, the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)), the promoter of the σ⁷⁰-type plastid rRNA gene (Prrn), the promoter of the psbA gene (encoding the photosystem-II reaction center protein D1) (PpsbA), the promoter of the psbD gene (encoding the photosystem-II reaction center protein D2) (PpsbD), the promoter of the psaA gene (encoding an apoprotein of photosystem I) (PpsaA), the promoter of the ATPase alpha subunit gene (PatpA), and promoter of the RuBisCo large subunit gene (PrbcL), and any combination thereof (See, e.g., De Cosa et al. Nat. Biotechnol. 19:71-74 (2001); Daniell et al. BMC Biotechnol. 9:33 (2009); Muto et al. BMC Biotechnol. 9:26 (2009); Surzycki et al. Biologicals 37:133-138 (2009)).

### Targeting

In some embodiments of the invention, the heterologous polynucleotides of the invention (e.g., the synthetic crTCA cycle polynucleotides described herein, polynucleotides encoding polypeptides for feeding the products of the synthetic cr TCA cycle into the Calvin Benson pathway, the SOR polynucleotides, the aquaporin polynucleotides, polynucleotides encoding inhibitors of cwII, and the like) can be transformed into the nucleus or into, for example, the chloroplast using standard techniques known in the art of plant transformation.

Thus, in some embodiments, one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, f) glyoxylate carboligase and/or (g) tartronic semialdehyde reductase can be transformed into and expressed in the nucleus and the polypeptides produced remain in the cytosol. In other embodiments, the one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, f) glyoxylate carboligase, and/or (g) tartronic semialdehyde reductase can be transformed into and expressed in the nucleus and the polypeptides can be targeted to the chloroplast. Thus, in particular embodiments, the one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, f) glyoxylate carboligase and/or (g) tartronic semialdehyde reductase can be operably associated with at least one targeting nucleotide sequence encoding a signal peptide that targets the polypeptides to the chloroplast.

In other embodiments, the heterologous polynucleotide encoding a superoxide reductase (SOR) can be operably associated with a targeting nucleotide sequence encoding a signal peptide that targets the heterologous SOR to the cytosol, cytosolic membrane (e.g., cytosolic surface of the plasma-membrane and other endogenous membranes including the nuclear envelope and endoplasmic reticulum), chloroplast, cell wall, peroxisome, mitochondria, and/or periplasm.

A signal sequence may be operably linked at the N- or C-terminus of a heterologous nucleotide sequence or nucleic acid molecule. Signal peptides (and the targeting nucleotide sequences encoding them) are well known in the art and can be found in public databases such as the “Signal Peptide Website: An Information Platform for Signal Sequences and Signal Peptides.” (www.signalpeptide.de); the “Signal Peptide Database” (proline.bic.nus.edu.sg/spdb/index.html) (Choo et al., BMC Bioinformatics 6:249 (2005) (available on www.biomedcentral.com/1471-2105/6/249/abstract); ChloroP (www.cbs.dtu.dk/services/ChloroP/; predicts the presence of chloroplast transit peptides (cTP) in protein sequences and the location of potential cTP cleavage sites); LipoP (www.cbs.dtu.dk/services/LipoP/; predicts lipoproteins and signal peptides in Gram negative bacteria); MITOPROT (ihg2.helmholtz-muenchen.de/ihg/mitoprot.html; predicts mitochondrial targeting sequences); PlasMit (gecco.org.chemie.uni-frankfurt.de/plasmit/index.html; predicts mitochondrial transit peptides in Plasmodium falciparum); Predotar (urgi.versailles.inra.fr/predotar/predotar.html; predicts mitochondrial and plastid targeting sequences); PTS1 (mendel.imp.ac.at/mendeljsp/sat/pts1/PTS1predictor.jsp; predicts peroxisomal targeting signal 1 containing proteins); SignalP (www.cbs.dtu.dk/services/SignalP/; predicts the presence and location of signal peptide cleavage sites in amino acid sequences from different organisms: Gram-positive prokaryotes, Gram-negative prokaryotes, and eukaryotes). The SignalP method incorporates a prediction of cleavage sites and a signal peptide/non-signal peptide prediction based on a combination of several artificial neural networks and hidden Markov models; and TargetP (www.cbs.dtu.dk/services/TargetP/); predicts the subcellular location of eukaryotic proteins—the location assignment is based on the predicted presence of any of the N-terminal presequences: chloroplast transit peptide (cTP), mitochondrial targeting peptide (mTP) or secretory pathway signal peptide (SP)). (See also, von Heijne, G., Eur J Biochem 133 (1) 17-21 (1983); Martoglio et al. Trends Cell Biol 8 (10):410-5 (1998); Hegde et al. Trends Biochem Sci 31(10):563-71 (2006); Dultz et al. J Biol Chem 283(15):9966-76 (2008); Emanuelsson et al. Nature Protocols 2(4) 953-971 (2007); Zuegge et al. 280(1-2):19-26 (2001); Neuberger et al. J Mol Biol, 328(3):567-79 (2003); and Neuberger et al. J Mol Biol. 328(3):581-92 (2003)).

Exemplary signal peptides include, but are not limited to those provided in Table 1.

TABLE 1
Amino acid sequences of representative signal peptides.

| Source | Sequence | Target |
|---|---|---|
| Rubisco small subunit | MASSVLSSAAVATRSNVAQANMVAPFTGLKSAASFPVSR | chloroplast |
| (tobacco) | KQNLDITSIASNGGRVQC (SEQ ID NO: 82) | |
| Saccharomyces | MLSLRQSIRFFKPATRTLCSSRYLL (SEQ ID NO: 83) | mitochondria |
| cerevisiae cox4 | | |
| Arabidopsis aconitase | MYLTASSSASSSIIRAASSRSSSLFSFRSVLSPSVSSTSPSSLL | mitochondria |
| | ARRSFGTISPAFRRWSHSFHSKPSPFRFTSQIRA (SEQ ID | |
| | NO: 84) | |
| Yeast aconitase | MLSARSAIKRPIVRGLATV (SEQ ID NO: 85) | mitochondria |
| Arabisopsis proline- | MRILPKSGGGALCLLFVFALCSVAHS (SEQ ID NO: 86) | cell |
| rich protein 2 | | wall/secretory |
| (AT2G21140) | | pathway |
| PTS-2 (conserved in | RLX₅HL (SEQ ID NO: 87) | peroxisome |
| eukaryotes) | MRLSIHAEHL (SEQ ID NO: 88) | |
| | SKL | |
| Arabidopsis | MLRTVSCLASRSSSSLFFRFFRQFPRSYMSLTSSTAALRVPSRNLR | chloroplast |
| presequence protease1 | RISSPSVAGRRLLLRRGLRIPSAAVRSVNGQFSRLSVRA (SEQ ID | |
| (AT3G19170) | NO: 89) | |
| Chlamydomonas | MALVARPVLSARVAASRPRVAARKAVRVSAKYGEN (SEQ ID | |
| reinhardtii-(Stroma- | NO: 90) | |
| targeting cTPs: | MQALSSRVNIAAKPQRAQRLVVRAEEVKA (SEQ ID NO: 91) | |
| photosystem I (PSI) | MQTLASRPSLRASARVAPRRAPRVAVVTKAALDPQ (SEQ ID | |
| subunits P28, P30, P35 | NO: 92) | |
| and P37, respectively) | MQALATRPSAIRPTKAARRSSVVVRADGFIG (SEQ ID NO: 93) | |
| C. reinhardtii- | MAFALASRKALQVTCKATGKKTAAKAAAPKSSGVEFYGPNRAK | chloroplast |
| chlorophyll a/b protein | WLGPYSEN (SEQ ID NO: 94) | |
| (cabII-1) | | |
| C. reinhardtii- | MAAVIAKSSVSAAVARPARSSVRPMAALKPAVKAAPVAAPAQA | chloroplast |
| Rubisco small subunit | NQMMVWT (SEQ ID NO: 95) | |
| C. reinhardtii- | MAAMLASKQGAFMGRSSFAPAPKGVASRGSLQVVAGLKEV | chloroplast |
| ATPase-γ | (SEQ ID NO: 96) | |
| Arabisopsis thaliana | CVVQ (SEQ ID NO: 97) | membrane |
| abscisic acid receptor | | |
| PYL10 | | |

X₅ means any five amino acids can be present in the sequence to target the protein to the peroxisome (e.g. RLAVAVAHL).

Thus, in representative embodiments of the invention, a heterologous polynucleotide encoding a polypeptide having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, f) glyoxylate carboligase and/or (g) tartronic semialdehyde reductase and/or a heterologous polynucleotide encoding an archaeon SOR to be expressed in a plant, plant cell, plant part can be operably linked to a chloroplast targeting sequence encoding a chloroplast signal peptide, optionally wherein said chloroplast signal peptide is encoded by an amino acid sequence that includes, but is not limited to, the amino acid sequence of SEQ ID NO:82, SEQ ID NO:90, SEQ ID NO:91, SEQ ID NO:92, SEQ ID NO:93, SEQ ID NO:94, SEQ ID NO:95, or SEQ ID NO:96.

In other embodiments of the invention, a heterologous polynucleotide encoding a SOR to be expressed in a plant, plant part or plant cell can be operably linked to a mitochondrial targeting sequence encoding a mitochondrial signal peptide, optionally wherein said mitochondrial signal peptide is encoded by an amino acid sequence that includes, but is not limited to, the amino acid sequence of SEQ ID NO:83, SEQ ID NO:84, or SEQ ID NO:85.

In further embodiments, a heterologous polynucleotide encoding a SOR to be expressed in a plant, plant part or plant cell can be operably linked to a cell wall targeting sequence encoding a cell wall signal peptide, optionally wherein said cell wall signal peptide is encoded by an amino acid sequence that includes, but is not limited to, the amino acid sequence of SEQ ID NO:86.

In still further embodiments of the invention, a heterologous polynucleotide encoding a SOR to be expressed in a plant, plant part or plant cell can be operably linked to a peroxisomal targeting sequence encoding a peroxisomal signal peptide, optionally wherein said peroxisomal signal peptide is encoded by an amino acid sequence that includes, but is not limited to, the amino acid sequence of SEQ ID NO:87, SEQ ID NO:88, or Ser-Lys-Leu (SKL).

In some embodiments, a heterologous polynucleotide encoding a SOR and/or an aquaporin, to be expressed in a plant, plant part or plant cell can be operably linked to a membrane targeting sequence encoding a membrane signal peptide, optionally wherein said membrane signal peptide is encoded by an amino acid sequence that includes, but is not limited to, the amino acid sequence of SEQ ID NO:97. In some embodiments, wherein when the heterologous polynucleotide encoding a SOR is targeted to a membrane, the SOR can be either linked directly to the membrane or to the membrane via a linkage to a membrane associated protein. In representative embodiments, a membrane associated protein includes but is not limited to the plasma membrane NADH oxidase (RbohA) (for respiratory burst oxidase homolog A) (Keller et al. The Plant Cell Online 10: 255-266 (1998)), annexin1 (ANN1) from Arabidopsis thaliana (Laohavisit et al. Plant Cell Online 24: 1522-1533 (2012)), and/or the nitrate transporter CHL1 (AtNRT1.1) (Tsay et al. “The Role of Plasma Membrane Nitrogen Transporters in Nitrogen Acquisition and Utilization,” In, The Plant Plasma Membrane 19:223-236 Springer Berlin/Heidelberg (2011)).

Targeting to a membrane is similar to targeting to an organelle. Thus, specific sequences on a protein (targeting sequences or motifs) can be recognized by a transporter, which then imports the protein into an organelle or in the case of membrane association, the transporter can guide the protein to and associate it with a membrane. Thus, for example, a specific cysteine residue on a C-terminal motif of a protein can be modified posttranslation where an enzyme (prenyltransferases) then attaches a hydrophobic molecule (geranylgeranyl or farnesyl) (See, e.g., Running et al. Proc Natl Acad Sci USA 101: 7815-7820 (2004); Maurer-Stroh et al. Genome Biology 4:212 (2003)). This hydrophobic addition guides and associates the protein to a membrane (in case of the cytosol, the membrane would be the plasma membrane or the cytosolic site of the nuclear membrane (Polychronidou et al. Molecular Biology of the Cell 21: 3409-3420 (2010)). More specifically, in representative embodiments, a protein prenyltransferase can catalyze the covalent attachment of a 15-carbon farnesyl or 20-carbon geranylgeranyl isoprenoid to C-terminal cysteines of selected proteins carrying a CaaX motif where C=cysteine; a=aliphatic amino acid; x=any amino acid. For plants, this motif most often is CVVQ (SEQ ID NO:97). The addition of prenyl groups facilitates membrane association and protein-protein interactions of the prenylated proteins.

In still other embodiments of the invention, a signal peptide can direct a polypeptide of the invention to more than one organelle (e.g., dual targeting). Thus, in some embodiments, a signal peptide that can target a polypeptide of the invention to more than one organelle is encoded by an amino acid sequence that includes, but is not limited to, the amino acid sequence of SEQ ID NO:89.

In addition to promoters operably linked to a heterologous polynucleotide of the invention, an expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences, translation termination sequences, and polyadenylation signal sequences, as described herein.

Thus, in some embodiments of the present invention, the expression cassettes can include at least one intron. An intron useful with this invention can be an intron identified in and isolated from a plant to be transformed and then inserted into the expression cassette to be used in transformation of the plant. As would be understood by those of skill in the art, the introns as used herein comprise the sequences required for self excision and are incorporated into the nucleic acid constructs in frame. An intron can be used either as a spacer to separate multiple protein-coding sequences in one nucleic acid construct, or an intron can be used inside one protein-coding sequence to stabilize the mRNA. If they are used within a protein-coding sequence, they are inserted “in-frame” with the excision sites included.

Non-limiting examples of introns useful with the present invention can be introns from the RuBisCO small subunit (rbcS) gene, the RuBisCO large subunit (rbcL) gene, the actin gene, the nitrate reductase gene (nr), the duplicated carbonic anhydrase gene 1 (Tdca1), the psbA gene, the atpA gene, or any combination thereof.

In some embodiments of the invention, an expression cassette can comprise an enhancer sequence. Enhancer sequences can be derived from, for example, any intron from any highly expressed gene. In particular embodiments, an enhancer sequence usable with this invention includes, but is not limited to, the nucleotide sequence of ggagg (e.g., ribosome binding site).

An expression cassette also can optionally include a transcriptional and/or translational termination region (i.e., termination region) that is functional in plants, yeast or bacteria. A variety of transcriptional terminators are available for use in expression cassettes and are responsible for the termination of transcription beyond the heterologous polynucleotide of interest and correct mRNA polyadenylation. The termination region may be native to the transcriptional initiation region, may be native to the operably linked nucleotide sequence of interest, may be native to the host cell, or may be derived from another source (i.e., foreign or heterologous to the promoter, the nucleotide sequence of interest, the host cell, or any combination thereof). Non-limiting examples of transcriptional terminators useful for plants can be a CAMV 35S terminator, a tml terminator, a nopaline synthase terminator and/or a pea rbcs E9 terminator, a RubisCo small subunit gene 1 (TrbcS1) terminator, an actin gene (Tactin) terminator, a nitrate reductase gene (Tnr) terminator, and/or aa duplicated carbonic anhydrase gene 1 (Tdca1) terminator.

Further non-limiting examples of terminators useful with this invention for expression of the heterologous polynucleotides of the invention or other heterologous polynucleotides of interest in algae include a terminator of the psbA gene (TpsbA), a terminator of the psaA gene (encoding an apoprotein of photosystem I) (TpsaA), a terminator of the psbD gene (TpsbD), a RuBisCo large subunit terminator (TrbcL), a terminator of the σ⁷⁰-type plastid rRNA gene (Trrn), and/or a terminator of the ATPase alpha subunit gene (TatpA).

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to a plant, plant part and/or plant cell expressing the marker and thus allows such a transformed plant, plant part, and/or plant cell to be distinguished from that which does not have the marker. Such a nucleotide sequence may encode either a selectable or screenable marker, depending on whether the marker confers a trait that can be selected for by chemical means, such as by using a selective agent (e.g., an antibiotic, herbicide, or the like), or whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding aadA (i.e., spectinomycin and streptomycin resistance), a nucleotide sequence encoding neo (i.e., kanamycin resistance), a nucleotide sequence encoding aphA6 (i.e., kanamycin resistance), a nucleotide sequence encoding nptII (i.e., kanamycin resistance), a nucleotide sequence encoding bar (i.e., phosphinothricin resistance), a nucleotide sequence encoding cat (i.e., chloramphenicol resistance), a nucleotide sequence encoding badh (i.e., betaine aldehyde resistance), a nucleotide sequence encoding egfp, (i.e., enhanced green fluorescence protein), a nucleotide sequence encoding gfp (i.e., green fluorescent protein), a nucleotide sequence encoding luc (i.e., luciferase), a nucleotide sequence encoding ble (bleomycin resistance), a nucleotide sequence encoding ereA (erythromycin resistance), and any combination thereof.

Further examples of selectable markers useful with the invention include, but are not limited to, a nucleotide sequence encoding an altered 5-enolpyruvylshikimate-3-phosphate (EPSP) synthase, which confers resistance to glyphosate (Hinchee et al., (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding Bla that confers ampicillin resistance; or a nucleotide sequence encoding aequorin which may be employed in calcium-sensitive bioluminescence detection (Prasher et al. (1985) Biochem. Biophys. Res. Comm. 126:1259-1268), and/or any combination thereof. One of skill in the art is capable of choosing a suitable selectable marker for use in an expression cassette of this invention.

An expression cassette comprising a heterologous polynucleotide of the invention (e.g., polynucleotide(s) encoding polypeptides of the synthetic crTCA cycle, glyoxylate carboligase, tartronic semialdehyde reductase, SOR, aquaporin and/or a polynucleotide encoding an inhibitor of cwII), also can optionally include polynucleotides that encode other desired traits. Such desired traits can be polynucleotides which confer high light tolerance, increased drought tolerance, increased flooding tolerance, increased tolerance to soil contaminants, increased yield, modified fatty acid composition of the lipids, increased oil production in seed, increased and modified starch production in seeds, increased and modified protein production in seeds, modified tolerance to herbicides and pesticides, production of terpenes, increased seed number, and/or other desirable traits for agriculture or biotechnology.

In particular embodiments, an expression cassette of this invention can further comprise an archaeal rubrerythrin reductase for conversion of hydrogen peroxide to water. Rubrerythrin reductase is an iron-dependent peroxidase that functions in vivo to remove the peroxide produced by superoxide reductase. Thus, a further embodiment of the invention includes a stably transformed plant comprising an expression cassette that comprises a SOR and a rubrerythrin reductase. In some embodiments, the SOR and rubrerythrin reductase are co-localized (i.e., they are expressed and targeted to the same or similar position in the transformed cell).

In some embodiments, an archaeal rubrerythrin reductase can be from Pyrococcus furiosus. In further embodiments, an archaeal rubrerythrin reductase can be optionally encoded by the nucleotide sequence of:

(SEQ ID NO: 98)
atggtcgtga aaagaacaat gactaaaaag ttcttggaag aagcctttgc aggcgaaagc
atggcccata tgaggtattt gatctttgcc gagaaagctg aacaagaagg atttccaaac
atagccaagc tgttcagggc aatagcttac gcagagtttg ttcacgctaa aaaccacttc
atagctctag gaaaattagg caaaactcca gaaaacttac agatgggaat agagggagaa
acgttcgaag ttgaggaaat gtacccagta tacaacaaag ccgcagaatt ccaaggagaa
aaggaagcag ttagaacaac ccactatgct ttagaggcgg agaagatcca cgctgaactc
tatagaaagg caaaagagaa agctgagaaa ggggaagaca ttgaaataaa gaaagtttac
atatgcccaa tctgtggata caccgctgtt gatgaggctc cagaatactg tccagtttgt
ggagctccaa aagaaaagtt cgttgtcttt gaatga

In still further embodiments, an archaeal rubrerythrin reductase can optionally comprise, consist essentially of, or consist of the amino acid sequence of:

(SEQ ID NO: 99)
MVVKRTMTKKFLEEAFAGESMAHMRYLIFAEKAEQEGFPNIAKLFRAIAYA
EFVHAKNHFIALGKLGKTPENLQMGIEGETFEVEEMYPVYNKAAEFQGEKE
AVRTTHYALEAEKIHAELYRKAKEKAEKGEDIEIKKVYICPICGYTAVDEA
PEYCPVCGAPKEKFVVFE

Such polynucleotides can be stacked with any combination of nucleotide sequences to create plants, plant parts and/or plant cells having the desired phenotype. Stacked combinations can be created by any method including, but not limited to, any conventional methodology (e.g., cross breeding for plants), or by genetic transformation. If stacked by genetic transformation, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

By “operably linked” or “operably associated,” it is meant that the indicated elements are functionally related to each other, and are also generally physically related. Thus, the term “operably linked” or “operably associated” as used herein, refers to nucleotide sequences on a single nucleic acid molecule that are functionally associated. Therefore, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Any plant (or groupings of plants, for example, into a genus or higher order classification) can be employed in practicing this invention including an angiosperm, a gymnosperm, a monocot, a dicot, a C3, C4, CAM plant, a microalgae, and/or a macroalgae.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell.

In some embodiments of this invention, a plant, plant part or plant cell can be from a genus including, but not limited to, the genus of Camelina, Sorghum, Gossypium, Brassica, Allium, Armoracia, Poa, Agrostis, Lolium, Festuca, Calamogrostis, Deschampsia, Spinacia, Beta, Pisum, Chenopodium, Helianthus, Pastinaca, Daucus, Petroselium, Populus, Prunus, Castanea, Eucalyptus, Acer, Quercus, Salix, Juglans, Picea, Pinus, Abies, Lemna, Wolffia, Spirodela, Oryza or Gossypium.

In other embodiments, a plant, plant part or plant cell can be from a species including, but not limited to, the species of Camelina alyssum (Mill.) Thell., Camelina microcarpa Andrz. ex DC., Camelina rumelica Velen., Camelina sativa (L.) Crantz, Sorghum bicolor (e.g., Sorghum bicolor L. Moench), Gossypium hirsutum, Brassica oleracea, Brassica rapa, Brassica napus, Raphanus sativus, Armoracia rusticana, Allium sative, Allium cepa, Populus grandidentata, Populus tremula, Populus tremuloides, Prunus serotina, Prunus pensylvanica, Castanea dentate, Populus balsamifer, Populus deltoids, Acer Saccharum, Acer nigrum, Acer negundo, Acer rubrum, Acer saccharinurn, Acer pseudoplatanus or Oryza sativa. In additional embodiments, the plant, plant part or plant cell can be, but is not limited to, a plant of, or a plant part, or plant cell from wheat, barley, oats, turfgrass (bluegrass, bentgrass, ryegrass, fescue), feather reed grass, tufted hair grass, spinach, beets, chard, quinoa, sugar beets, lettuce, sunflower (Helianthus annuus), peas (Pisum sativum), parsnips (Pastinaca sativa), carrots (Daucus carota), parsley (Petroselinum crispum), duckweed, pine, spruce, fir, eucalyptus, oak, walnut, or willow. In particular embodiments, the plant, plant part and/or plant cell can be from Camelina sativa.

In further embodiments, a plant and/or plant cell can be an algae or algae cell from a class including, but not limited to, the class of Bacillariophyceae (diatoms), Haptophyceae, Phaeophyceae (brown algae), Rhodophyceae (red algae) or Glaucophyceae (red algae). In still other embodiments, a plant and/or plant cell can be an algae or algae cell from a genus including, but not limited to, the genus of Achnanthidium, Actinella, Nitzschia, Nupela, Geissleria, Gomphonema, Planothidium, Halamphora, Psammothidium, Navicula, Eunotia, Stauroneis, Chlamydomonas, Dunaliella, Nannochloris, Nannochloropsis, Scenedesmus, Chlorella, Cyclotella, Amphora, Thalassiosira, Phaeodactylum, Chrysochromulina, Prymnesium, Thalassiosira, Phaeodactylum, Glaucocystis, Cyanophora, Galdieria, or Porphyridium. Additional nonlimiting examples of genera and species of diatoms useful with this invention are provided by the US Geological Survey/Institute of Arctic and Alpine Research at westerndiatoms.colorado.edu/species.

Any nucleotide sequence to be transformed into a plant, plant part and/or plant cell can be modified for codon usage bias using species specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications for the nucleotide sequences for selection are determined by comparing the species specific codon usage table with the codons present in the native polynucleotide sequences. In those embodiments in which each of codons in native polynucleotide sequence for selection are sufficiently used, then no modifications are needed (e.g., a frequency of more than 30% for a codon used for a specific amino acid in that species would indicate no need for modification). In other embodiments, wherein up to 3 nucleotides have to be modified in the polynucleotide sequence, site-directed mutagenesis can be used according to methods known in the art (Zheng et al. Nucleic Acids Res. 32:e115 (2004); Dammai, Meth. Mol. Biol 634:111-126 (2010); Davis and Vierstra. Plant Mol. Biol. 36(4): 521-528 (1998)). In still other embodiments, wherein more than three nucleotide changes are necessary, a synthetic nucleotide sequence can be generated using the same codon usage as the highly expressed genes that were used to develop the codon usage table.

The term “transformation” as used herein refers to the introduction of a heterologous polynucleotide into a cell. Transformation of a plant, plant part, plant cell, yeast cell and/or bacterial cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome. The phrase “a stably transformed plant, plant part, and/or plant cell expressing said one or more polynucleotide sequences” and similar phrases used herein, means that the stably transformed plant, plant part, and/or plant cell comprises the one or more polynucleotide sequences and that said one or more polynucleotide sequences are functional in said stably transformed plant, plant part, and/or plant cell.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols that are well known in the art.

A heterologous polynucleotide encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, f) glyoxylate carboligase and/or (g) tartronic semialdehyde reductase; a heterologous polynucleotide encoding an archaeal SOR; a heterologous polynucleotide encoding an aquaporin and/or an inhibitor of cwII as described herein; and/or functional fragments thereof (e.g., a functional fragment of the nucleotide sequences of SEQ ID NOs:3, 6, 9, 12, 15, 18, 21, 24, 27, 30, 33, 36, 39, 42, 45, 48, 51, 53, 55, 57, 59, 61, 63, 65, 67, 69, 71, 72, 74, 76, 78, 80, 99, 101, 103, 105 to 111, and/or any combination thereof or the amino acid sequences of SEQ ID NOs:1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 54, 56, 58, 60, 62, 64, 66, 68, 70, 74, 75, 77, 79, 81 to 97, 99, 101, 103, and/or any combination thereof) can be introduced into a cell of a plant by any method known to those of skill in the art. In some embodiments of the invention, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al., (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)). General guides to the transformation of yeast include Guthrie and Fink (1991) (Guide to yeast genetics and molecular biology. In Methods in Enzymology, (Academic Press, San Diego) 194:1-932) and guides to methods related to the transformation of bacteria include Aune and Aachmann (Appl. Microbiol Biotechnol 85:1301-1313 (2010)).

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

In some embodiments, when a plant part or plant cell is stably transformed, it can then be used to regenerate a stably transformed plant comprising one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, f) glyoxylate carboligase and/or (g) tartronic semialdehyde reductase, a heterologous polynucleotide encoding an archaeal SOR, a heterologous polynucleotide encoding an aquaporin and/or an inhibitor of cwII as described herein, and/or other polynucleotides of interest as described herein, and/or any combination thereof in its genome. Means for regeneration can vary from plant species to plant species, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

The particular conditions for transformation, selection and regeneration of a plant can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

Accordingly, in some aspects of the invention, a stably transformed plant, plant part and/or plant cell is provided, which comprises in its genome one or more recombinant nucleic acid molecules/heterologous polynucleotides of the invention and has increased carbon fixation and/or increased biomass production, reduced reactive oxygen species, increased CO₂ uptake and/or assimilate partitioning directed into fruits and seeds of said stably transformed plant. Thus, in some embodiments, the invention provides a stably transformed plant, plant part and/or plant cell comprising one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase, which when expressed results in the stably transformed plant, plant part or plant cell having increased carbon fixation and/or increased biomass production. In other aspects, the invention provides a stably transformed plant, plant part and/or plant cell comprising in its genome one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase, which when expressed results in the stably transformed plant, plant part or plant cell having increased carbon fixation and/or increased biomass production. In representative embodiments, the one or more heterologous polynucleotides encoding polypeptides having the enzyme activity of (a) to (e) and/or (a) to (g) are expressed in the nucleus and are targeted to the chloroplast and/or are expressed in the chloroplast.

In additional aspects, the invention provides a stably transformed plant, plant part and/or plant cell comprising in its genome one or more heterologous polynucleotides encoding polypeptides having the enzyme activity of a) succinyl CoA synthetase, (b) 2-oxoglutarate:ferredoxin oxidoreductase, (c) 2-oxoglutarate carboxylase, (d) oxalosuccinate reductase, and (e) isocitrate lyase, and a heterologous polynucleotide encoding an archaeal SOR, wherein the stably transformed plant, plant part or plant cell has increased carbon fixation and/or increased biomass production and reduced reactive oxygen species as compared to a control. In other aspects, the invention provides a stably transformed plant, plant part and/or plant cell comprising in its genome one or more heterologous polynucleotides encoding polypeptides having the enzyme activity of a) succinyl CoA synthetase, (b) 2-oxoglutarate:ferredoxin oxidoreductase, (c) 2-oxoglutarate carboxylase, (d) oxalosuccinate reductase, and (e) isocitrate lyase, and a heterologous polynucleotide encoding an aquaporin, wherein the stably transformed plant, plant part or plant cell having increased carbon fixation and/or increased biomass production and increased CO₂ uptake as compared to a control. In still other aspects, the invention provides a stably transformed plant comprising in its genome one or more heterologous polynucleotides encoding polypeptides having the enzyme activity of a) succinyl CoA synthetase, (b) 2-oxoglutarate:ferredoxin oxidoreductase, (c) 2-oxoglutarate carboxylase, (d) oxalosuccinate reductase, and (e) isocitrate lyase, and a heterologous polynucleotide encoding an inhibitor of cwII, wherein the stably transformed plant has increased carbon fixation and/or increased biomass production and increased assimilate partitioning into fruits and seeds as compared to a control. In representative embodiments, the heterologous polynucleotide encoding an archaeal SOR can be expressed in the nucleus and targeted to the chloroplast, mitochondria, peroxisome, cell wall and/or cell membrane (e.g., cytosolic membrane (e.g., cytosolic surface of the plasma-membrane and other endogenous membranes including the nuclear envelope and endoplasmic reticulum)) or can be expressed in the chloroplast.

In further embodiments, the invention provides a stably transformed plant comprising in its genome one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase, a heterologous polynucleotide encoding an archaeal SOR and a heterologous polynucleotide encoding an inhibitor of cwII, wherein expression of said polynucleotides results in the stably transformed plant having increased carbon fixation and/or increased biomass production, reduced reactive oxygen species and increased assimilate partitioning into fruits and seeds as compared to a control.

The invention further provides a stably transformed plant comprising in its genome one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase, a heterologous polynucleotide encoding an archaeal SOR and a heterologous polynucleotide encoding an aquaporin, wherein expression of said polynucleotides results in the stably transformed plant having increased carbon fixation and/or increased biomass production, reduced reactive oxygen species and increased CO₂ uptake as compared to a control.

In additional embodiments, the invention provides a stably transformed plant comprising in its genome one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase, a heterologous polynucleotide encoding an archaeal SOR, a heterologous polynucleotide encoding an aquaporin, and a heterologous polynucleotide encoding an inhibitor of cwII, wherein expression of said polynucleotides results in the stably transformed plant having increased carbon fixation and/or increased biomass production, reduced reactive oxygen species, increased CO₂ uptake and increased assimilate partitioning into fruits and seeds as compared to a control.

In further embodiments, the invention provides a stably transformed plant comprising in its genome one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase, a heterologous polynucleotide encoding an aquaporin and a heterologous polynucleotide encoding an inhibitor of cwII, wherein expression of said polynucleotides results in the stably transformed plant having increased carbon fixation and/or increased biomass production, increased CO₂ uptake, and increased assimilate partitioning into fruits and seeds as compared to a control.

In additional aspects, the invention provides a stably transformed plant, plant part and/or plant cell comprising in its genome one or more heterologous polynucleotides encoding polypeptides having the enzyme activity of a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase and a heterologous polynucleotide encoding an archaeal SOR, which when expressed results in the stably transformed plant, plant part or plant cell having increased carbon fixation and/or increased biomass production and reduced reactive oxygen species as compared to a control. In other aspects, the invention provides a stably transformed plant, plant part and/or plant cell comprising in its genome one or more heterologous polynucleotides encoding polypeptides having the enzyme activity of a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase, and a heterologous polynucleotide encoding an aquaporin, which when expressed results in the stably transformed plant, plant part or plant cell having increased carbon fixation and/or increased biomass production and increased CO₂ uptake as compared to a control. In still other aspects, the invention provides a stably transformed plant comprising in its genome one or more heterologous polynucleotides encoding polypeptides having the enzyme activity of a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase, and a heterologous polynucleotide encoding an inhibitor of cwII, wherein expression of said polynucleotides results in the plant having increased carbon fixation and/or increased biomass production and increased assimilate partitioning into fruits and seeds as compared to a control.

In further embodiments, the invention provides a stably transformed plant comprising in its genome one or more heterologous polynucleotides encoding polypeptides having the enzyme activity of a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase, a heterologous polynucleotide encoding an aquaporin and a heterologous polynucleotide encoding an inhibitor of cwII, wherein expression of said polynucleotides results in the stably transformed plant having increased carbon fixation and/or increased biomass production, increased CO₂ uptake, and increased assimilate partitioning into fruits and seeds as compared to a control.

In further embodiments, the invention provides a stably transformed plant comprising in its genome one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase, a heterologous polynucleotide encoding an archaeal SOR and a heterologous polynucleotide encoding an inhibitor of cwII, wherein expression of said polynucleotides results in the stably transformed plant having increased carbon fixation and/or increased biomass production, reduced reactive oxygen species and increased assimilate partitioning into fruits and seeds as compared to a control.

The invention further provides a stably transformed plant comprising in its genome one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase, a heterologous polynucleotide encoding an archaeal SOR and a heterologous polynucleotide encoding an aquaporin, wherein expression of said polynucleotides results in the stably transformed plant having increased carbon fixation and/or increased biomass production, reduced reactive oxygen species and increased CO₂ uptake as compared to a control.

In some embodiments, the invention provides a stably transformed plant comprising in its genome one or more heterologous, polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, (e) isocitrate lyase, (f) glyoxylate carboligase, and (g) tartronic semialdehyde reductase, a heterologous polynucleotide encoding an archaeal SOR, a heterologous polynucleotide encoding an aquaporin, and a heterologous polynucleotide encoding an inhibitor of cwII, wherein expression of said polynucleotides results in the stably transformed plant having increased carbon fixation and/or increased biomass production, reduced reactive oxygen species, increased CO₂ uptake and increased assimilate partitioning into fruits and seeds as compared to a control.

Additionally provided herein are seeds produced from the stably transformed plants of the invention, wherein said seeds comprise in their genome the one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase. In some embodiments, the seeds produced from the stably transformed plants of the invention further comprise in their genome one or more heterologous polynucleotides encoding polypeptides having the enzyme activity of glyoxylate carboligase and tartronic semialdehyde reductase. In other embodiments, the seeds produced from the stably transformed plants of the invention further comprise in their genome a heterologous polynucleotide encoding an archaeal SOR, a heterologous polynucleotide encoding an aquaporin, and/or a heterologous polynucleotide encoding an inhibitor of cwII.

The present invention further provides a product produced from the stably transformed plant, plant cell or plant part of the invention. In some embodiments, the product produced can include but is not limited to biofuel, food, drink, animal feed, fiber, and/or pharmaceuticals.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

As used herein, the terms “fragment” when used in reference to a polynucleotide will be understood to mean a nucleic acid molecule or polynucleotide of reduced length relative to a reference nucleic acid molecule or polynucleotide and comprising, consisting essentially of and/or consisting of a nucleotide sequence of contiguous nucleotides identical or almost identical (e.g., 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment according to the invention may be, where appropriate, included in a larger polynucleotide of which it is a constituent.

As used herein, a “functional” polypeptide or “functional fragment” is one that substantially retains at least one biological activity normally associated with that polypeptide. In particular embodiments, the “functional” polypeptide or “functional fragment” substantially retains all of the activities possessed by the unmodified peptide. By “substantially retains” biological activity, it is meant that the polypeptide retains at least about 50%, 55%, 60%, 65%, 70%, 75%, 80%, 85%, 90%, 91%, 92%, 93%, 94%, 95%, 96%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Thus, for example, a functional fragment of an archaeon SOR polypeptide is a polypeptide that retains at least 50% or more SOR activity.

An “isolated” nucleic acid molecule or nucleotide sequence or nucleic acid construct or double stranded RNA molecule of the present invention is generally free of nucleotide sequences that flank the nucleic acid of interest in the genomic DNA of the organism from which the nucleic acid was derived (such as coding sequences present at the 5′ or 3′ ends). However, the nucleic acid molecule of this invention can include some additional bases or moieties that do not deleteriously or materially affect the basic structural and/or functional characteristics of the nucleic acid molecule.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose. In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

As used herein, “complementary” polynucleotides are those that are capable of hybridizing via base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely or fully complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules either along the full length of the molecules or along a portion or region of the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least at about 50%, 60%, 70%, 80% or 90% of their nucleotides. In some embodiments, the two nucleic acid sequences can be complementary at least at about 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid molecule or nucleotide sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous polynucleotide includes a nucleotide sequence derived from and inserted into the same natural, original cell type, but which is present in a non-natural state, e.g. present in a different copy number, and/or under the control of different regulatory sequences than that found in the native state of the nucleic acid molecule.

As used herein, the terms “transformed” and “transgenic” refer to any plant, plant part, and/or plant cell that contains all or part of at least one recombinant (e.g., heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transgene” as used herein, refers to any nucleotide sequence used in the transformation of an organism. Thus, a transgene can be a coding sequence, a non-coding sequence, a cDNA, a gene or fragment or portion thereof, a genomic sequence, a regulatory element and the like. A “transgenic” organism, such as a transgenic plant, transgenic yeast, or transgenic bacterium, is an organism into which a transgene has been delivered or introduced and the transgene can be expressed in the transgenic organism to produce a product, the presence of which can impart an effect and/or a phenotype in the organism.

Different nucleotide sequences or polypeptide sequences having homology are referred to herein as “homologues.” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids, and/or proteins.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” means that two nucleotide sequences have at least 70%, 75%, 80%, 85%, 90% or 95% sequence identity. In some embodiments, the two nucleotide sequences can have at least 80%, 85%, 90%, 91%, 92%, 93%, 94%, 95%, 96%, 97%, 98%, 99% or 100% sequence identity. Thus, for example, a homolog of a polynucleotide of the invention can have at least about 80%, 85%, 90%, 91%, 92%, 93%, 94%, 95%, 96%, 97%, 98%, 99% or 100% sequence identity to, for example, a polynucleotide encoding a polypeptide having the enzyme activity of succinyl CoA synthetase, 2-oxoglutarate:ferredoxin oxidoreductase, 2-oxoglutarate carboxylase, oxalosuccinate reductase, isocitrate lyase, glyoxylate carboligase, tartronic semialdehyde reductase, a heterologous polynucleotide encoding an archaeal SOR, a heterologous polynucleotide encoding an aquaporin, and/or a heterologous polynucleotide encoding an inhibitor of cwII.

Two nucleotide sequences can also be considered to be substantially identical when the two sequences hybridize to each other under stringent conditions. A nonlimiting example of “stringent” hybridization conditions include conditions represented by a wash stringency of 50% formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window is well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo et al. (Applied Math 48:1073 (1988)). More particularly, preferred computer programs for determining sequence identity include but are not limited to the Basic Local Alignment Search Tool (BLAST) programs which are publicly available from National Center Biotechnology. Information (e.g., NCBI) at the National Library of Medicine, National Institute of Health, Bethesda, Md. 20894; see BLAST Manual, Altschul et al., e.g., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and for polynucleotide sequence BLASTN can be used to determine sequence identity.

Accordingly, the present invention further provides polynucleotides having substantial sequence identity (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99% and/or 100% identity) to the polynucleotides of the present invention (e.g., a polynucleotide encoding a polypeptide having the enzyme activity of succinyl CoA synthetase, 2-oxoglutarate:ferredoxin oxidoreductase, 2-oxoglutarate carboxylase, oxalosuccinate reductase, isocitrate lyase, glyoxylate carboligase, and/or tartronic semialdehyde reductase; a heterologous polynucleotide encoding an archaeal SOR; a heterologous polynucleotide encoding an aquaporin; and/or a heterologous polynucleotide encoding an inhibitor of cwII).

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### EXAMPLES

### Example 1

### The Synthetic crTCA Pathway Enzymes

Increasing the productivity of a C3 plant such as camelina to levels seen for C4 plants (e.g. corn) requires improving photosynthetic carbon fixation. One limiting factor is the oxygenase activity of the CO₂-fixing Ribulose 1,5 bisphosphate Carboxylase/Oxygenase (RUBISCO) that reduces the photosynthetic productivity by up to 30%. The present invention provides methods and compositions for improving carbon fixation in plants by introducing a synthetic carbon fixation pathway that is independent of RUBISCO but works in concert with the existing Calvin Benson cycle.

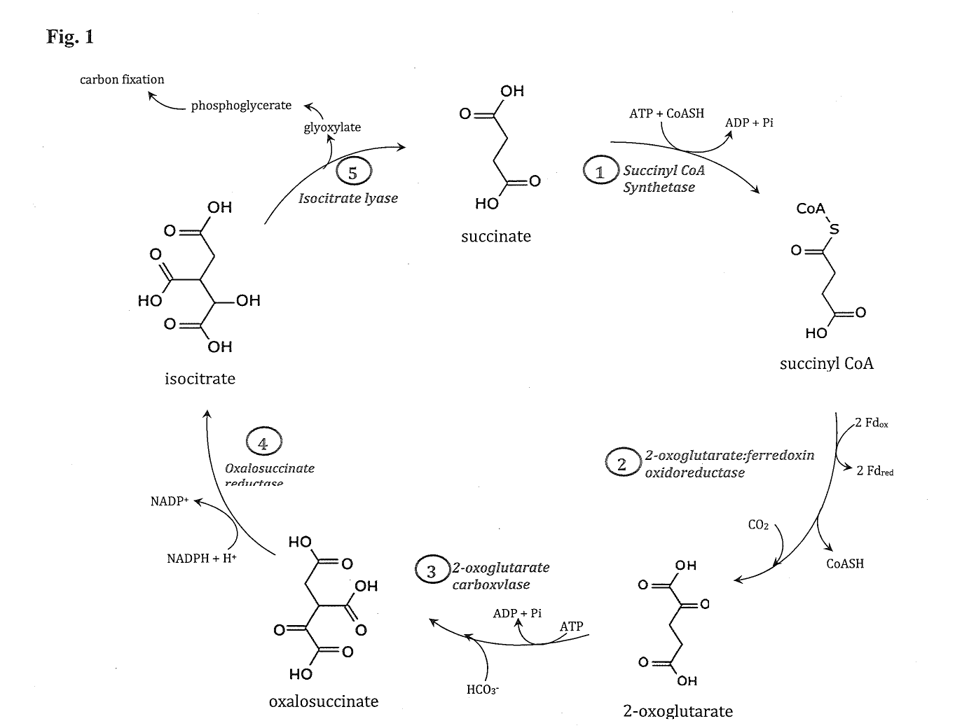

Specifically, this invention provides a “condensed reverse TCA (crTCA) cycle,” that employs a (1) succinyl-CoA synthetase for catalyzing the conversion of succinate to succinyl-CoA, (2) a 2-oxoglutarate:ferredoxin oxidoreductase for converting succinyl-CoA to 2-oxoglutarate (i.e., 2-ketoglutarate), (3) a 2-oxoglutarate carboxylase for converting 2-oxoglutarate to oxalosuccinate, (4) an oxalosuccinate reductase for converting oxalosuccinate to isocitrate, and (5) an isocitrate lyase for cleaving isocitrate into succinate and glyoxylate (FIG. 1).

The net product of the crTCA cycle is glyoxylate. In order to feed the assimilated carbon from glyoxylate into the Calvin Benson cycle, two additional enzymes can be used to first convert two glyoxylate molecules into tartronic-semialdehyde via glyoxylate carboligase, and then reduce tartronic-semialdehyde into glycerate using the tartronic-semialdehyde reductase. The resulting glycerate can then be phosphorylated by the chloroplastic glycerate kinase to glycerate phosphate, a Calvin Benson cycle intermediate, thus ensuring that the CO₂ fixed via the synthetic crTCA cycle increases carbon flux into the endogeneous assimilation cycle. It is noted that the crTCA cycle requires 4 ATP, 4 ferredoxin (Fd) and 2 NADPH for the conversion of 4 CO2 into 2 molecules of glyoxylate, which compares favorably to the energy and reductant requirements for the equivalent Calvin Benson cycle fixation (9 ATP, 6 NADPH) (Berg et al., 2010).

For generation of the synthetic crTCA cycle, specific enzymes were chosen from source bacteria based on the following criteria: (1) experimentally determined function of the enzyme, (2) target enzymes having the fewest subunits, and (3) in cases in which enzyme activity is unavailable, enzyme choice based on highest homology levels to characterized enzymes having the desired activity.

### Candidate Enzymes

For the succinyl CoA synthetase enzyme activity, the characterized Escherichia coli version of this enzyme can be used (e.g., SucC and SucD, NCBI Accession Nos: NC_{—}000913.2 (762,237 . . . 763,403), NC_{—}000913.2 (763,403 . . . 764,272)_NP_{—}415256.1 and NP_{—}415257.1) (Buck et al. J Gen Microbiol 132:1753-62 (1986)). Additional succinyl CoA synthetase versions that can also be used include those from Azotobacter vinelandii DJ, (NCBI Accession Nos. NC_{—}012560.1 (3,074,152 . . . 3,075,321), NC_{—}012560.1 (3,073,268 . . . 3,074,155, YP_{—}002800115.1 and YP_{—}002800114.1; Bradyrhizobium sp.BTAi1, (NCBI Accession Nos. NC_{—}009485.1 (393,292 . . . 394,488), NC_{—}009485.1 (394,545 . . . 395,429), YP_{—}001236586.1 and YP_{—}001236587.1); and/or Azospirillum sp. B510, (NCBI Accession Nos. NC_{—}013854.1 (2,941,010 . . . 2,942,206), NC_{—}013854.1 (2,942,208 . . . 2,943,083), YP_{—}003449758.1 and YP_{—}003449759.1) (See, e.g., the nucleotide sequences of SEQ ID NOs:3, 6, 9 and/or 12; the amino acid sequences of SEQ ID NOs:1, 2, 4, 5, 7, 8, 10 and/or 11).

Oxoglutarate:ferredoxin oxidoreductase (OOR) is an important enzyme in the crTCA cycle that enables the cycle to function in the reverse direction (Buchanan and Arnon Photosynth Res 24:47-53 (1990). There are two types of OORs, a two subunit version expressed in the anaerobic phototrophic bacterium Chlorobium limicola (Buchanan and Arnon Photosynth Res 24:47-53 (1990)) and the aerobic halophile Halobacterium salinarum (Kerscher and Oesterhelt Eur J Biochem 116:587-94 (1981)) and a four subunit version expressed in anaerobic sulfur reducing bacteria such as Sulfurimonas denitrificans (Hugler et al. J. Bacteriol 187:3020-7 (2005)). Because the crTCA cycle is meant to function in plants using oxygenic photosynthesis and limiting enzyme subunits can simplify the generation of the transgenic plant lines, the two subunit version of OOR from an aerobic bacterium can be used. Based on homology to the biochemically characterized H. salinarum OOR, a two subunit OOR was selected with good identity from the aerobic bacterium Paenibacillus larvae subsp. larvae B-3650 ((NCBI Accession Nos. PlarlB_{—}020100012680 and PlarlB_{—}020100012675, NZ_ADZY02000226.1 (7,939 . . . 9,687), NZ_ADZY02000226.1 (7,085 . . . 7,951), ZP_{—}09070120.1 and ZP_{—}09070119.1). Additional versions of OOR that could be used include the following: Halobacterium sp. NRC-1 korA, korB, (NCBI Accession Nos. NC_{—}002607.1 (856,660 . . . 858,582), NC_{—}002607.1 (855,719 . . . 856,657), AAG19514.1 and AAG19513.1, NP_{—}280034.1 and NP_{—}280033.1); Hydrogenobacter thermophilus TK-6 korA, korB, ((NCBI Accession Nos. NC_{—}013799.1 (997,525 . . . 999,348), NC_{—}013799.1 (996,624 . . . 997,511), YP_{—}003432752.1 and YP_{—}003432751.1; Bacillus sp. M3-13 Bm3-1_{—}010100005806, Bm3-1_{—}010100005801, NZ_ACPC01000013.1 (932Dz,668), NZ_ACPC01000013.1 (65 . . . 931), ZP_{—}07708142.1 and ZP_{—}07708141.1); Haladaptatus paucihalophilus DX253 (NCBI Accession Nos. ZOD2009_{—}10775, ZOD2009-10770, contig00009, whole genome shotgun sequence NZ_AEMG01000009.1 (157,678DZ59,432), NZ_AEMG01000009.1 (156,818 . . . 157,681), ZP_{—}08044530.1 and ZP_{—}08044529.1); and/or Magnetococcus sp. (NCBI Accession Nos. MC-1 Mmc1_{—}1749, Mmc1_{—}1750, NC_{—}008576.1 (2,161,258 . . . 2,162,979), NC_{—}008576.1 (2,162,976 . . . 2,163,854), YP_{—}865663.1 and YP_{—}865664.1). (See, e.g., the nucleotide sequences of SEQ ID NOs:15, 18, 21, 24, 27 and/or 30; or the amino acid sequences of SEQ ID NOs: 13, 14, 16, 17, 19, 20, 22, 23, 25, 26, 28 and/or 29).

The prediction of in vivo function for the five step crTCA cycle is reliant on the energy utilizing step catalyzed by 2-oxoglutarate carboxylase in order to provide an overall negative ΔG for the cycle (Bar-Even et al. Proc Natl Acad Sci USA 107:8889-94 (2010)). Currently, the only characterized version of a 2-oxoglutarate carboxylase is from the thermophilic chemoautotrophic bacterium Hydrogenobacter thermophilus TK-6, which optimally functions at 80° C. (Aoshima and Igarashi Mol Microbiol 62:748-59 (2006)). Homology analysis using the H. thermophilus korA; and korB subunit sequences was able to identify subunits from a nitrite-oxidizing bacterium Candidatus Nitrospira defluvii having high identity (pycA, and pycB; NCBI Accession Nos. NC_{—}014355.1 (1,174,721DZ,176,652), NC_{—}014355.1 (1,176,781DZ,178,199), YP_{—}003796887.1 and YP_{—}003796888.1). These genes are identified as subunits of pyruvate carboxylase in the N. defluvii genome; however, protein modeling analysis determined that the N. defluvii carboxylase has high specificity for oxoglutarate. Additional versions of 2-oxoglutarate carboxylase that could be used include, for example, Hydrogenobacter thermophilus TK-6 cfiA, cfiB, (NCBI Accession Nos. NC_{—}013799.1 (1,271,487 . . . 1,273,445), NC_{—}013799.1 (1,273,469DZ,274,887), YP_{—}003433044.1 and YP_{—}003433045.1 and its modified version (see, e.g., SEQ ID NOs:37-42)); Thiocystis violascens DSM198 (NCBI Accession Nos. ThiviDRAFT_{—}1483, ThiviDRAFT_{—}1486, whole genome shotgun sequence, ctg263, NZ_AGFC01000013.1 (61,879 . . . 63,297) and (63,889 . . . 65,718), ZP_{—}08925050.1 and ZP_{—}08925052.1); Mariprofundus ferrooxydans PV-1 (NCBI Accession Nos. SPV1_{—}07811, SPV1_{—}07816, NZ_AATS01000007.1 whole genome shotgun sequence, 1099921033908 (81,967 . . . 83,385) and (83,475 . . . 85,328), ZP_{—}01452577.1 AND ZP_{—}01452578.1); and/or Pseudomonas stutzeri ATCC14405 (NCBI Accession Nos. PstZobell_{—}14412 and PstZobell_{—}14407, CCUG 16156 contig00098, whole genome shotgun sequence AGSL01000085.1 (52,350 . . . 53,765) and (50,522 . . . 52,339), EHY78621.1 and EHY78620.1). (See, e.g., the nucleotide sequences of SEQ ID NOs: 33, 36, 39, 42, 45, 48 and/or 51; or the amino acid sequences of SEQ ID NOs: 31, 32, 34, 35, 37, 38, 40, 41, 43, 44, 46, 47, 49 and/or 50).

The next enzyme in the cycle, oxalosuccinate reductase, has also been characterized from H. thermophilus (Aoshima and Igarashi Mol Microbiol 62:748-59 (2006)). We identified a further oxalosuccinate reductase from the soil bacterium Acinetobacter baumannii (NCBI Accession Nos. ACICU_{—}02687, NC_{—}010611.1 (2,855,563 . . . 2,856,819) YP_{—}001847346.1), which has high homology to oxalosuccinate reductase from H. thermophilus. Additional versions of oxalosuccinate reductase that also could be used include the following: Chlorobium limicola DSM 245 Cl-idh, (NCBI Accession Nos. AB076021.1, BAC00856.1); Kosmotoga olearia TBF 19.5.1 (NCBI Accession Nos. Kole_{—}1227, NC_{—}012785.1 (1,303,493DZ,304,695), YP_{—}002940928.1); Marine gamma proteobacterium HTCC2080 (NCBI Accession Nos. MGP2080_{—}11238, 1100755000543, whole genome shotgun sequence NZ_AAVV01000002.1 (123,681 . . . 124,934), ZP_{—}01625318.1); and/or Nitrosococcus halophilus Nc4 (NCBI Accession Nos. Nhal_{—}2539, NC_{—}013960.1 (2,610,547Dz,611,815), YP_{—}003528006.1). (See, e.g., the nucleotide sequences of SEQ ID NOs: 53, 55, 57, 59 and/or 61; or the amino acid sequences of SEQ ID NOs: 52, 54, 56, 58 and/or 60).

For the isocitrate lyase step, the biochemically characterized version from Corynebacterium glutamicum ((NCBI Accession Nos. NCgl2248, NC_{—}003450.3 (2,470,741 . . . 2,472,039) NP_{—}601531.1) can be used (Reinscheid et al. J Bacteriol 176:474-83 (1994)). Additional versions of isocitrate lyase that could be used include the following: Gordonia alkanivorans NBRC 16433 aceA (locus tag=GOALK_{—}050_{—}00390), contig: GOALK050, whole genome shotgun sequence (NCBI Accession Nos. NZ_BACI01000050.1 (37,665 . . . 38,960), ZP_{—}08765259.1); Nocardia farcinica IFM 10152 aceA (locus tag=nfa52300), NC_{—}006361.1 (5,525,226 . . . 5,526,515) YP_{—}121446.1; Rhodococcus pyridinivorans AK37 (NCBI Accession Nos. AK37_{—}18248, contig53, whole genome shotgun sequence NZ_AHBW01000053.1 (20,169 . . . 21,458), ZP_{—}09310682.1); and/or Rhodococcus jostii RHA1 (NCBI Accession Nos. RHA1_ro02122, NC_{—}008268.1 (2,230,309Dz,231,598), YP_{—}702087.1). (See, e.g., the nucleotide sequences of SEQ ID NOs: 63, 65, 67, 69 and/or 71; or the amino acid sequences of SEQ ID NOs: 62, 64, 66, 68 and/or 70).

Initial demonstration of function of the novel synthetic crTCA cycle, will be accomplished by expressing the identified enzymes in E. coli, purifying the expressed enzymes and showing in an in vitro assay system that the appropriate crTCA cycle reactions occur. The genes encoding the crTCA cycle enzymes, which have been analyzed for optimal codon usage in camelina, and synthetic versions made as necessary, are then introduced into an expression construct for transformation into a plant such as camelina.

In order for the crTCA cycle to function in plants to enhance photosynthetic carbon fixation, the glyoxylate generated by the crTCA cycle can be converted to a metabolite that flows into the Calvin Benson Cycle. Thus, a heterologous polynucleotide sequence encoding a polypeptide having the enzyme activity of glyoxylate carboligase (e.g., nucleotide sequences of SEQ ID NO:100 and/or SEQ ID NO:101) and a heterologous polynucleotide sequence encoding a polypeptide having the enzyme activity of tartronic-semialdehyde reductase (e.g., nucleotide sequences of SEQ ID NO:102 and/or SEQ ID NO:103) can be transformed into the plant (e.g., camelina) nuclear genome and targeted to the chloroplast using chloroplast targeting sequences. Thus, the synthetic crTCA cycle can be introduced into plants that also express at least a polynucleotide encoding a polypeptide having the enzyme activity of glyoxylate carboligase and a nucleotide sequence encoding a polypeptide having the enzyme activity of tartronic-semialdehyde reductase.

### Example 2

### Expression of the crTCA Pathway in E. coli

The crTCA pathway will be expressed first in E. coli to verify CO₂ fixation. The genes encoding the crTCA cycle selected enzymes will then be analyzed for optimal codon usage in camelina and synthetic versions made as necessary. These will then be introduced into camelina singly or as a polygene cluster construct.

The specific enzymes to be used initially in the crTCA pathway include succinyl-CoA synthetase from E. coli version (SucC, SucD) (Buck et al. J Gen Microbiol. 132(6):1753-62 (1986)) (see, e.g., the nucleotide sequence of SEQ ID NO:3 (amino acid sequences of SEQ ID NO:1 and SEQ ID NO:2)). An oxoglutarate:ferredoxin oxidoreductase (OOR) from Paenibacillus larvae subsp. larvae B-3650 (see, e.g., the nucleotide sequence of SEQ ID NO:24; amino acid sequences of SEQ ID NO:22 and SEQ ID NO:23) will be used.

Using a mesophilic carboxylase enzyme from a nitrite-oxidizing bacterium, Candidatus Nitrospira defluvii, amino acids were identified as supporting specificity for oxoglutarate. Then the corresponding amino acid substitutions were made in a thermophilic Hydrogenobacter thermophilis TK-6 2-oxoglutarate carboxylase resulting in a thermophilic 2-oxoglutarate carboxylase that can function at lower temperatures than the native H. themophilus TK-6 2-oxoglutarate carboxylase. Specifically, the large subunit from the 2-oxoglutarate carboxylase polypeptide (cfiA) from Hydrogenobacter thermophilus TK-6 was modified at residue 203 to be alanine (A) instead of methionine (M), at residue 205 to be valine (V) instead of phenylalanine (F), at residue 234 to be methionine (M) instead of threonine (T), at residue 236 to be threonine (T) instead of isoleucine (I), at residue 240 to be leucine (L) instead of methionine (M), at residue 274 to be arginine (R) instead of glutamic acid (E) and/or at residue 288 to be glutamine (Q) instead of aspartic acid (D) as shown, for example, in the amino acid sequences of SEQ ID NO:38 and SEQ ID NO:41 and the corresponding codon changes as shown, for example, in the nucleotide sequences of SEQ ID NO:39 or SEQ ID NO:42.

Oxalosuccinate reductase from Chlorobium limicola DSM 245 (see, e.g., the nucleotide sequence of SEQ ID NO:53; amino acid sequence of SEQ ID NO:52), Marine gamma proteobacterium HTCC2080 (see, e.g., the nucleotide sequence of SEQ ID NO:59; amino acid sequence of SEQ ID NO:58), Kosmotoga olearia TBF 19.5.1 (see, e.g., the nucleotide sequence of SEQ ID NO:55; amino acid sequence of SEQ ID NO:54), and/or Nitrosococcus halophilus Nc4 (see, e.g., the nucleotide sequence of SEQ ID NO:61; amino acid sequence of SEQ ID NO:60) can be used in the synthetic crTCA cycle.

An isocitrate lyase from Corynebacterium glutamicum will be used (see, e.g., the nucleotide sequence of SEQ ID NO:63; amino acid sequence of SEQ ID NO:62) (Reinscheid et al. J Bacteriol. 176(12):3474-83 (1994)).

Construction of crTCA Expression Vectors for Recombinant Production in E. Coli

Polynucleotides encoding the crTCA enzymes described above are amplified with sequence specific primers that contain restriction sites appropriate for cloning into an expression plasmid (e.g., pET-21b and pET-28a expression plasmids and/or the Qiagen pQE-1 vector), to enable expression of C- and N-terminal His-tagged proteins, respectively. Each construct is sequenced to ensure that no mutations have been introduced during cloning. A crTCA cycle expression construct can then be generated expressing all 5 crTCA cycle enzymes (non-His tagged) coordinately so crTCA cycle function in E. coli can be assessed.

Thus, polynucleotide sequences corresponding to each candidate protein were synthesized by GenScript and optimized for expression in E. coli (codon optimization). The polynucleotide sequences were delivered on the pUC57 plasmid either in the EcoRV site or in other sites as determined by GenScript.

The synthesized polynucleotide sequences were PCR amplified using the BioRad iProof™ high fidelity polymerase. The forward primer started with the ATG of each polynucleotide sequence and the reverse primer incorporated an appropriate restriction site for cloning PCR products into expression vector pQE-1. Forward primers for some polynucleotide sequences required HPLC purification to ensure that the full ATG was present on the 5′ end of the primer and therefore present in the cloned polynucleotide sequences.

Purified PCR products were phosphorylated and then ligated into pQE-1. The resulting pQE-1 constructs were used to transform E. coli strain XL-1. Plasmid DNA was isolated and sequenced to confirm: a) polynucleotide insert is correctly positioned in pQE-1, b) polynucleotide sequence is correct and free of mutations. Confirmed constructs were used to transform expression strain E. coli M15

Small scale cultures (30 ml LB) of E. coli M15 containing pQE-1 constructs were grown to mid log phase, then samples were harvested for SDS-PAGE analysis. Expression conditions were then optimized, then large scale cultures (1 L) were grown for protein purification with affinity chromatography. The pQE-1 His-tag system was confirmed to be functioning correctly by the Western Blot.

### Small-Scale Protein Expression Protocol:

pQE1:crTCA cycle constructs comprising the polynucleotide sequences of interest (e.g., encoding crTCA polypeptides) and pQE1-only controls were used to transform E. coli M15 containing the pREP plasmid. Aliquots from overnight cultures were used to inoculate 30 ml LB broth. Cell growth was monitored spectrophotometrically (600 nm), and when mid log growth phase was evident (OD₆₀₀=0.6 to 0.8), protein expression was induced by the addition of IPTG (0.2 mM final concentration). Cell cultures were incubated at 30° C. for 6 h and with agitation (175 rpm). After the 6 hr induction period, 1 ml samples were collected and cells were pelleted by centrifugation at 4° C., 8,079×g. Spent media was discarded and the cell pellet was resuspended in 50 μl of 50 mM potassium phosphate buffer pH 7.0 and 0.5 μl each of freshly prepared 1M benzamidine and 1M DTT. A 2 μl aliquot of the resuspended cell pellet was mixed with 10 μl 2× dye and 8 μl dH₂O. The mixture was incubated at 100° C. for 15 min to denature proteins, which were then analyzed by SDS-PAGE (12.5% polyacrylamide) for 35 min at 200V.

Recombinant crTCA Enzyme Purification

Cell pellets containing the recombinant crTCA cycle proteins were suspended in 50 mM potassium phosphate buffer, pH 8.0 containing 1 mM benzamidine-HCl. The cell suspension was passed through a French pressure cell (1,100 lb/in²) three times. The lysed suspension was centrifuged at 15,000×g for 60 min at 4° C. to remove cell debris. The supernatant was filtered through 0.45 μm syringe filters to further remove debris. The filtered extract was applied to a 5 ml HisTrap HP Nickel Sepharose™ affinity column (GE Healthcare Life Sciences) and washed with five column volumes of wash buffer (50 mM sodium phosphate buffer, pH 8.0, 20 mM imidazole). The binding buffer used was 50 mM sodium phosphate buffer, pH 8.0, 10 mM imidizole, and the elution buffer was 50 mM sodium phosphate buffer, pH 8.0, 250 mM imidizole. Elution was done via a linear gradient from 0% to 100% elution buffer. All fractions were visualized on 12.5% SDS-polyacrylamide gels. Following affinity chromatography, the samples containing recombinant protein were pooled and dialyzed using a 10,000 Da molecular weight cutoff (MWCO) dialysis cassette against 50 mM Tris-HCl, pH 8.0, to remove unwanted imidazole from the fractions. Final protein concentrations were estimated using Bio-Rad's Bradford assay.

### Protein Expression Results

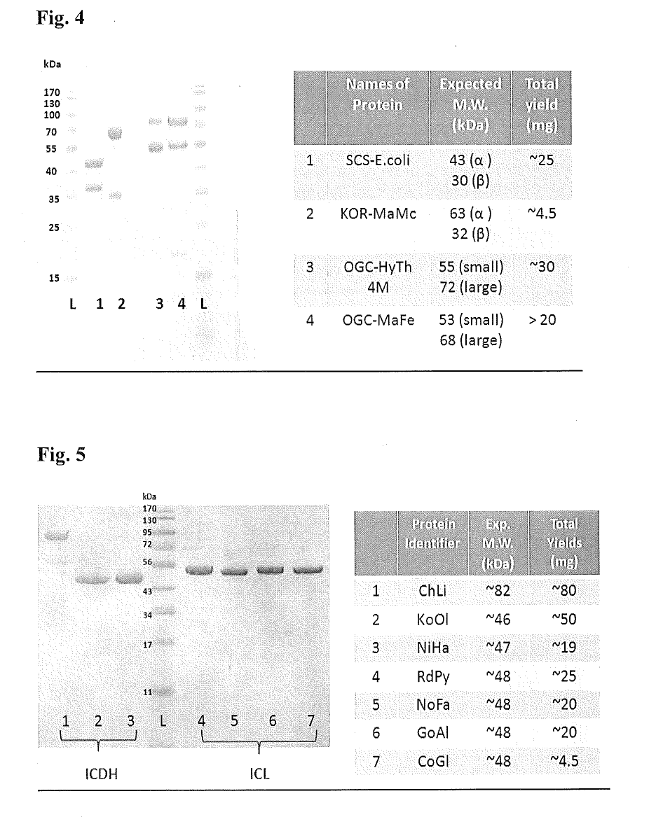

A 12.5% SDS-polyacrylamide gel showing purified crTCA Cycle Enzyme 1 (Succinyl CoA Synthetase (ScS)), Enzyme 2 (2-Oxoglutarate Ferredoxin Oxidoreductase (KOR)), and Enzyme 3 (2-Oxoglutarate Carboxylase (OGC)) is presented in FIG. 4.

A 12.5% SDS-polyacrylamide gel showing purified crTCA Cycle Enzyme 4 variants (Oxalosuccinate Reductase (ICDH)) and Enzyme 5 variants (Isocitrate Lyase (ICL)) is presented in FIG. 5.

(1) crTCA Cycle Reaction #1: Succinyl CoA Synthetase

### Brief Assay Description:

the succinyl CoA synthetase (SCS) assay is a spectrophotometric method that measures the increase of absorbance at 230 nm in response to thioester formation.

### Assay Method:

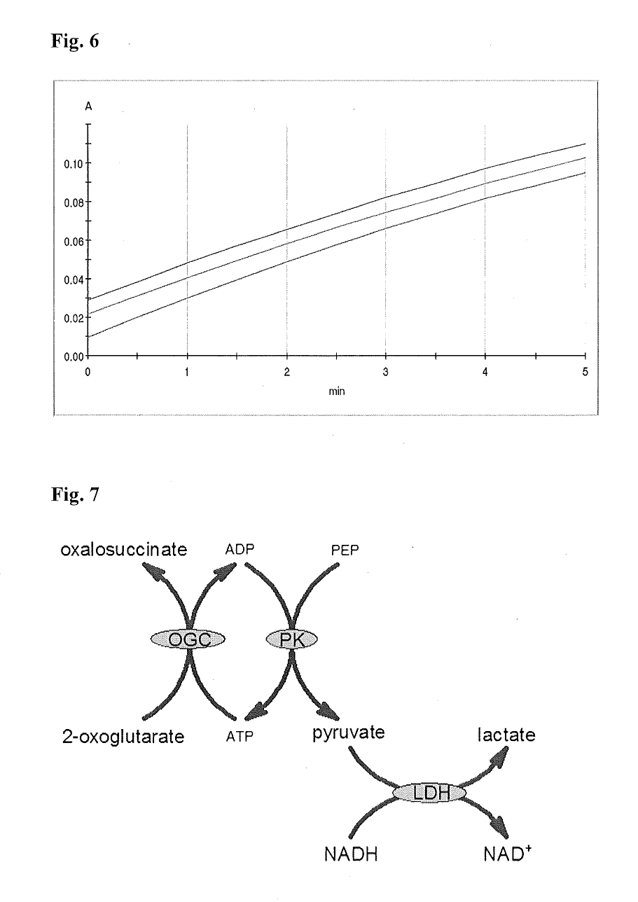

The standard reaction solution consisted of 10 mM sodium succinate, 10 mM MgCl₂, 0.1 mM CoA, 0.1 mM DTT, 0.4 mM nucleotide ATP and 0.1 M KCl in 50 mM Tris-HCl (pH 7.4). The reaction was started with the addition of purified E. coli succinyl coA synthetase. The reaction was monitored in a spectrophotometer set at 230 nm at room temperature. A spectrum showing the SCS assay is provided in FIG. 6. The specific activity of the SCS enzyme is provided in Table 2, below.

TABLE 2
Calculated specific activity

| Cycle | | Specific activity |
|---|---|---|
| Enzyme | Source organism | (μmol/min/mg) |
| Succinyl CoA | Escherichia coli strain K-12 | 11.8 ± 0.4 |
| Synthetase | substr. MG1655 | |
| (SCS) | | |

(2) crTCA Cycle Reaction #2: 2-Oxodlutarate:Ferredoxin Oxidoreductase (OGOR)

### Brief Assay Description:

The assay for the forward reaction for OGOR is a LC-MS based assay in which 2-oxoglutarate is measured directly by LC-ESI-QTOF-MS.

### Assay Method:

The final reaction mixture contains 10 mM NH₄Ac (pH 7.0), 0.5 mM MgCl₂, 1 mM DTT, 20 mM NH₄HCO₃, 1 mM succinyl CoA and proteins (OGOR and ferredoxin). The gas phase in the quartz cell is replaced with argon. The reaction is initiated by addition of succinyl-CoA. After incubating at room temperature for 30 minutes, the reaction is stopped by heating the reaction mixture to 100° C. for 10 minutes, followed by centrifugation at 14,000 rpm for 30 minutes. The supernatant is stored for further LC-MS analysis.

(3) crTCA Cycle Reaction #3: 2-Oxoglutarate Carboxylase (OGC)

### Brief Assay Description:

The 2-Oxoglutarate Carboxylase (OGC) assay is a discontinuous spectrophotometric assay in which the ATPase activity is determined indirectly at 340 nm (measuring NADH oxidation). See FIG. 7.

### Assay Method:

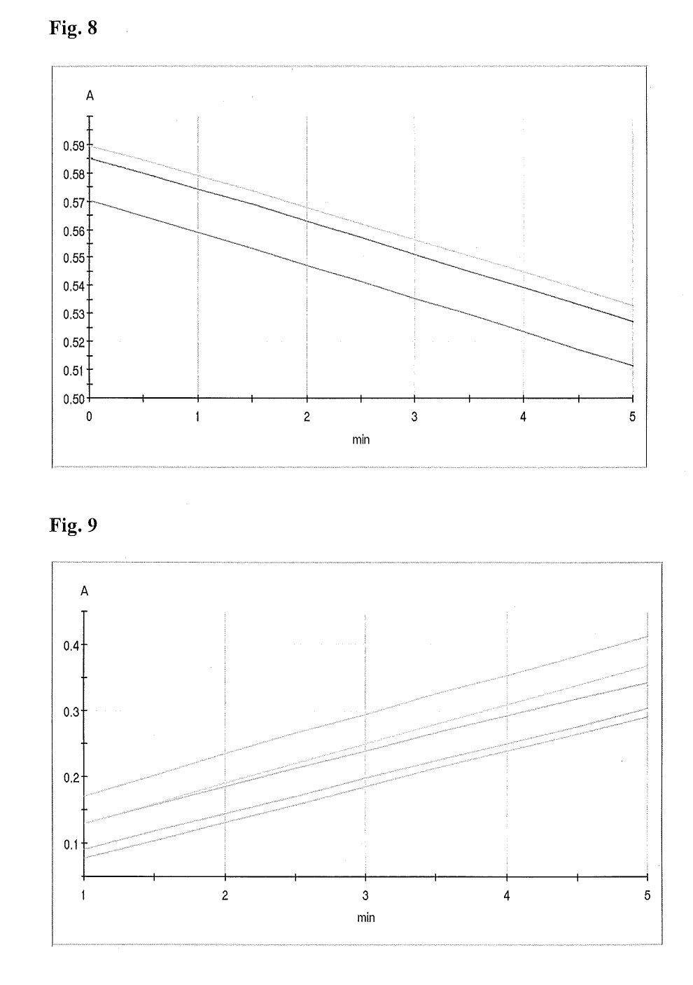

The reaction mixture is composed of 100 mM PIPES (pH 6.5), 5 mM MgCl₂, mM 2-oxoglutarate, 50 mM NaHCO₃, 5 mM ATP. The reaction was initiated by addition of OGC. After incubating for 35 min at 65° C., the reaction mixture was cooled down to room temperature. Then 0.1 mM β-NADH, 2 mM phosphoenolpyruvate (PEP) and PK/LDH were added to the reaction mixture, in which NADH oxidation was monitored spectrophotometrically at 340 nm. The amount of ADP produced was determined using a standard curve. A spectrum showing the OGC assay is provided in FIG. 8 and the specific activity of the OCG enzyme is provided in Table 3, below.

TABLE 3
Calculated specific activity.

| Cycle | | Specific activity |
|---|---|---|
| Enzyme | Source organism | (nmol/min/mg) |
| 2-oxoglutarate | Hydrogenobacter thermophilus TK-6 | 73 ± 4 |
| carboxylase | (with 4 amino acid replacements) | |
| (OGC) | | |

(4) crTCA Cycle Reaction #4: Oxalosuccinate Reductase

### Brief Assay Description:

The assay for oxalosuccinate reductase (isocitrate dehydrogenase, ICDH) is a continuous assay. The dehydrogenase activity of this enzyme is monitored spectrophotometrically at 340 nm, measuring the reduction of NADP⁺.

### Assay Method:

The reaction mixture is composed of 50 mM Tris (pH 7.4), 10 mM MgCl₂, 100 mM KCl, 4 mM isocitrate, 4 mM β-NADP⁺ and the recombinant ICDH enzyme. The reaction was initiated by addition of enzyme and monitored by NADP⁺ reduction at 340 nm. A spectrum showing the ICDH assay (from Nitrosococcus halophilus Nc4) is provided in FIG. 9 and the specific activity of the ICDH enzyme from Chlorobium limicola, Kosmotoga olearia TBF 19.5.1, and Nitrosococcus halophilus Nc4 is provided in Table 4, below.

TABLE 4
Calculated specific activity.

| Cycle | | Specific activity |
|---|---|---|
| Enzyme | Source organism | (μmol/min/mg) |
| Isocitrate | Chlorobium limicola | 11.7 ± 0.8 |
| dehydro- | Kosmotoga olearia TBF 19.5.1 | 0.42 ± 0.01 (RT) |
| genase | | 67 (65° C.) |
| (ICDH) | Nitrosococcus halophilus Nc4 | 19 ± 1 |

(5) crTCA Cycle Reaction #5: Isocitrate Lyase

### Brief Assay Description:

The assay for isocitrate lyase (ICL) is a continuous spectrophotometric rate determination in which ICL converts isocitrate to succinate and glyoxylate. The glyoxylate is chemically converted to glyoxylate phenylhydrazone in the presence of phenylhydrazine. The glyoxylate phenylhydrazone is measured at 324 nm.

### Assay Method:

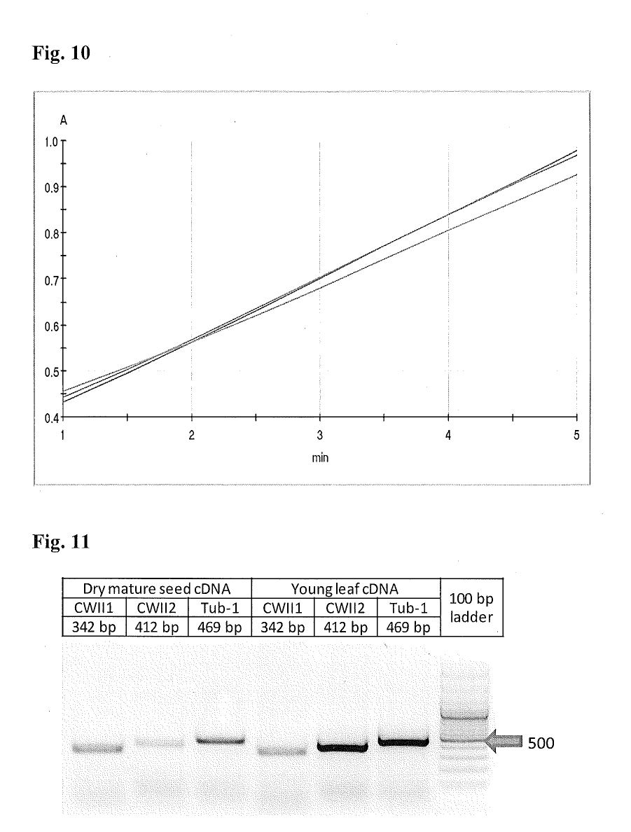

The reaction mixture contains 30 mM imidazole (pH 6.8), 5 mM MgCl₂, 1 mM EDTA, 4 mM phenylhydrazine and 10 mM isocitrate. The reaction was performed at room temperature. After adding ICL, the reaction was continuously monitored at 324 nm. A spectrum showing the ICL assay (from Rhodococcus pyridinivorans AK37) is provided in FIG. 10 and the specific activity of the ICDH enzyme from Corynebacterium glutamicum ATCC 13032, Gordonia alkanivorans NBRC 16433, Nocardia farcinica IFM 10152 and Rhodococcus pyridinivorans AK37 is provided in Table 5, below

TABLE 5
Calculated specific activity.

| Cycle | | Specific activity |
|---|---|---|
| Enzyme | Source organism | (μmol/min/mg) |
| Isocitrate | Corynebacterium glutamicum | 1.26 |
| | ATCC 13032 | |
| lyase | Gordonia alkanivorans NBRC 16433 | 0.31 ± 0.04 |
| (ICL) | Nocardia farcinica IFM 10152 | 10.0 ± 0.3 |
| | Rhodococcus pyridinivorans AK37 | 4.9 ± 0.4 |

### Example 3

### Expression of the Synthetic crTCA Pathway in Camelina sativa

The oilseed crop Camelina sativa (L.) Crantz has been naturalized to almost all of the United States (United States Department of Agriculture USDA, N.R.C.S. Plant Database. 2011). It is grown in rotation either as an annual summer crop or biannual winter crop. It is adapted to a wide range of temperate climates on marginal land, is drought and salt tolerant, and requires very little water or fertilizer. Its seeds have a high oil content (≧40%) that can be extracted by energy efficient cold pressing. The remaining omega-3 fatty acid-rich meal has been approved by the FDA for inclusion in livestock feed. A further advantage is that camelina does not compete for land with food crops and produces feed for livestock as well as productivity (and jobs) on unfarmed land. Camelina further has a short life cycle and can produce up to four generations per year in greenhouses.

Camelina sativa will be genetically engineered to express a new synthetic pathway (crTCA) to increase photosynthetic CO₂ assimilation in the leaves and other useful characteristics. This pathway will be integrated with other transgenes to increase the CO₂ concentration inside the chloroplast (CO₂-transporter AQP1), increase photosynthetic efficiency by reducing reactive oxygen species (archea superoxide reductase) and/or to increase the export of the assimilated carbon from the leaves to the fruits and seeds.

As discussed above, the synthetic shortened version of the rTCA, which we term the condensed reverse TCA (crTCA) cycle, employs enzymes that have the activity of (1) a succinyl-CoA synthetase that catalyzes conversion of succinate to succinyl-CoA, (2) a 2-oxoglutarate:ferredoxin oxidoreductase that converts succinyl-CoA to 2-oxoglutarate, (3) a 2-oxoglutarate carboxylase that converts 2-oxoglutarate to oxalosuccinate, (4) an oxalosuccinate reductase that converts oxalosuccinate to isocitrate, and (5) an isocitrate lyase that cleaves isocitrate into succinate and glyoxylate (FIG. 1). Therefore, to increase photosynthetic CO₂ fixation a synthetic carbon fixation pathway (crTCA), as discussed above in Example 1 and Example 2, that could work in concert with the existing Calvin Benson cycle polynucleotides encoding polypeptides having the enzyme activity of succinyl-CoA synthetase, 2-oxoglutarate:ferredoxin oxidoreductase, 2-oxoglutarate, oxalosuccinate reductase and isocitrate lyase will be introduced into camelina.

The glyoxylate generated by the crTCA cycle will ultimately be converted by two additional enzymes, glyoxylate carboligase and tartronic-semialdehyde reductase, to phosphoglycerate, which can then be used for carbon fixation in the Calvin Benson cycle, thereby increasing overall photosynthetic carbon fixation.

### Example 4

### Increasing CO₂ Uptake into the Chloroplast (AQP1)

Slow diffusion of CO2 across cell wall and inner chloroplast membrane limits photosynthetic rates (Flexas et al. Plant Cell Environ. 31(5):602-21 (2008); Tholen and Zhu. Plant Physiol. 156(1):90-105 (2011)). An approach to overcoming this limitation and increasing CO₂ uptake can be through the introduction into a plant of an aquaporin. An aquaporin with high similarity to human CO₂ porin (AQP1) has been identified in tobacco and shown to facilitate CO₂ membrane transport in plants (Uehlein et al. Nature. 425(6959):734-7 (2003); Uehlein et al. Plant Cell 20(3):648-57 (2008); Flexas et al. Plant J. 48(3):427-39 (2006)). This NtAQP1 is localized to the inner chloroplast envelope membrane as well as to mesophyll cell plasma membranes (Uehlein et al. Plant Cell 20(3):648-57 (2008)). Expression of an aquaporin such as NtAQP1 in camelina under a constitutive promoter (e.g., 35S constitutive promoter) should increase CO₂ conductivity to the site of fixation, resulting in increased carbon fixation (e.g., increased photosynthesis) and/or increased biomass production.

### Example 5

### Reducing Reactive Oxygen Species by Superoxide Reductase (SOR)

Oxidative damage by reactive oxygen species (ROS) as a result of plant metabolism and environmental stress reduces photosynthetic efficiency (Foyer and Noctor. Antioxid Redox Signal 11(4):861-905 (2009); Krieger-Liszkay et al. Physiol Plant. 142(1):17-25 (2011)). Antioxidant enzymes such as superoxide dismutases, peroxidases and catalases protect photosystems (Krieger-Liszkay et al. Physiol Plant. 142(1):17-25 (2011); Allen et al. Free Radic Biol Med. 23(3):473-9 (1997); Payton et al. J Exp Bot. 52(365):2345-54 (2001); Tseng et al. Plant Physiol Biochem 45(10-11):822-33 (2007)). Our research showed that expression in plant systems of a catalytically efficient superoxide reductase (SOR) from the hyperthermophilic archaeon Pyrococcus furiosus protects chlorophyll function in response to environmental stresses such as heat, high light, and drought (Im et al. Plant Physiol. 151(2):893-904 (2009); Im et al. FEBS Lett. 579(25):5521-6 (2005)). P. furiosus SOR will be expressed in camelina as well to reduce ROS levels and protect photosystem function.

### Example 6

### Increasing Sucrose Partitioning into Seeds (cwII RNAi)

The export of sugars occurs from photosynthesizing mesophyll cells through the cell wall into the phloem/companion cell complex, which carries sugars via mass flow to non-photosynthetic tissues. Phloem unloading occurs either via the cell wall (apoplasm) or via plasmodesmata (Koch, K., Curr Opin Plant Biol. 7(3):235-46 (2004); Ward et al. International Review of Cytology—a Survey of Cell Biology Vol 178:41-71 (1998)). Export and import through the apoplasm are controlled by the activity of cell wall invertase (cwI), which hydrolyzes sucrose into glucose and fructose and is regulated by a specific inhibitor protein (cwII) (Ward et al. International Review of Cytology—a Survey of Cell Biology Vol 178:41-71 (1998); Ruan et al. Molecular Plant. 3(6):942-955 (2010)). In general, low cell wall invertase activity increases sucrose export from the source tissue, and high cell wall invertase activity increases sucrose unloading into fruits and seeds/grains. Quantitative trait loci analysis for fruit size in tomato (Lin5), and grain size in rice (GIF1) and maize (MN1) identified mutations in cell-wall invertases that led to reduction in its activity in pedicel/fruit tissues (Wang et al. Nature Genetics 40(11):1370-1374 (2008); Fridman et al. Science. 305(5691):1786-1789 (2004); Cheng et al. Plant Cell. 8(6):971-983 (1996)) as key regulators for phloem unloading and therefore determinants of seed and fruit size. Fruit-specific suppression of the cell wall invertase inhibitor (cwII) in tomato and rice led to increases in net seed/grain weight of 22% and 10%, respectively (Wang et al. Nature Genetics 40(11):1370-1374 (2008); Jin et al. Plant Cell. 21(7):2072-89 (2009). Two general approaches have been used to modify sucrose flux: overexpression of cwI or repression of its inhibitor protein, cwII (Wang et al. Nature Genetics 40(11):1370-1374 (2008); Sonnewald et al. Plant J. 1(1):95-106 (1991); von Schaewen et al. Embo J 9(10):3033-44 (1990); Zanor et al. Plant Physiology 150(3):1204-1218 (2009); Jin et al. Plant Cell. 21(7):2072-89 (2009); Greiner et al. Nat Biotechnol. 17(7):708-11 (1999)). In the present invention, suppression of CwII in camelina via RNAi technology will be used to direct assimilate partitioning into fruit/seeds.

Thus, to identify a cwII, leaf tissue from Camelina sativa was sequenced using two multiplexed lanes on an Illumina GAIIx flow cell. Sequences for invertase inhibitors from Arabidopsis (thaliana and lyrata), tobacco, and tomato were BLASTed against assembled contigs from the camelina leaf RNA Seq reads. Each of the two Arabidopsis genes aligned to hit a single sequence, the long assembled contig with tblastn had percent identity ≧80% and with an e-value cutoff of 10⁻¹⁰. The sequences from tobacco and tomato only yielded hits once the identity threshold was reduced to 40%.

Based on the individual amino acid alignments with Arabidopsis and the ClustalW multiple-sequence alignments comparing Arabidopsis thaliana, Arabidopsis lyrata, and Camelina sativa contigs, the hits were considered to reliably represent cell wall invertase inhibitors in camelina and will be referred to from here on as putative sequences “CWII 1” and “CWII 2”.

RT-PCR using cDNA from dry mature camelina seeds and young leaf as well as CWII isoform specific primers revealed that both cwII isoforms are expressed in both tissues (FIG. 11). Based on the sequence alignments as discussed above, we generated isoform specific primers for cwII to characterize their expression in seeds. Primers to tubulin-1 were used as internal controls. Both isoforms are present in both tissues (leaf and seed), but it appears that the amount of cwII1 expressed in mature seeds is greater compared to cwII2, while mRNA abundance of cwII2 is greater in young leaves compared to cwII1. The promoter sequences of both CWII genes were identified for use in driving expression of the antisense/RNAi constructs.

Four fragments—one corresponding to pCWII1 and three corresponding to pCWII2—were sequenced. All four were confirmed to be valid TAIL-PCR products. All fragments contained the expected known portion of sequence as well as unknown sequence upstream. The TAIL-PCR for pCWII2 revealed 650 bp of previously unknown sequence upstream of the known segment of the gene. The TAIL-PCR for pCWII1 revealed only an additional ˜118 bp of previously unknown sequence upstream of the known segment of the gene. Based on the direct sequencing results, the identity of the CWII1 product was confirmed.

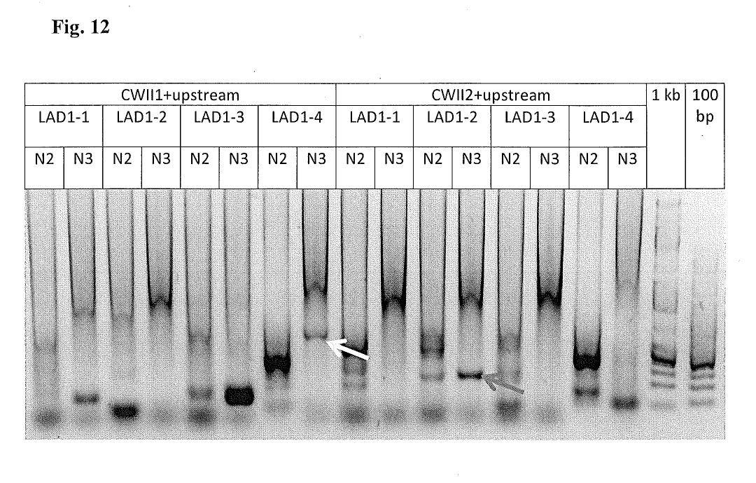

A longer fragment of the pCWII1 gene was identified with additional rounds of TAIL PCR (FIG. 12). Sequencing confirmed the likely function of a promoter as an upstream region of the CWII. In addition, a BLAST Search for sequences having some similarity to those from camelina yielded two cell wall/vacuolar inhibitors of fructosidase 1. Based on these results, we are confident that this represents a valid sequence upstream of the known coding sequences for CWII1 (see, SEQ ID NO:109, which includes promoter and coding sequence) and CWII2 (see, SEQ ID NO:110, which includes promoter and coding sequence)

### Promoter Analysis

First the start codon had to be identified from the total sequence. Because the template used thus far came from the RNASeq Analysis (PE 7), and the outermost primers were within that sequence, the beginning of the gene was not discovered until the first round of TAIL-PCR. For each of the sequences—especially CWII1—several “ATG” sites could be found close to the area where the beginning of the coding sequence was expected. To pinpoint this location, the total known sequence (including the ˜600 bp upstream) was aligned as a translated nucleotide BLAST against a protein database to determine the site from the Arabidopsis amino acid sequence.

The total known sequence (promoter and coding sequence) of CWII1 from camelina is as follows with the start codon boxed.

SEQ ID NO: 109
CTCAAAAATTAGCATTAAAAATTCTGTAAATGAACTTTAATAAATAGTATATATTTAATTAAAAAGCAATATTGA
AATTTTGAAAACCAAAAAAATGTATAGTAATTTTGAAATTCAAATCATTGCAGGAAATTAAATACATAGATGGTT
TTAGGCATAAATACACTTTCCATATCATGATCACTTGACTAATATTAATTTGGCATATTTATAATTTCATAGTAA
GATGTTATTTCAGTGTGGTCACAATATTAGACATTATATAATGTATATATAATTTATATTAGTGTTTTTGCCAAA
TTTGTTCTTGGATACTATAGAAACTAAAAAGATTAATAACCCAAACTAAAGAAATTTAAAAACATTCAAATTAAA
TTTTGATNGGACAATATCAATTTGGTGGTATATACTAAAATAAAAGTATATTACCTGAAAATATCAGAAATGATA
TATAGGTTTTTTATCCTTATTAAGAGATTTTGGTAAAGGCACGCCACCAATTCAATTATATATATACTGGTNNCG
GGCAGTACACAGACAAGACACACACACTTATAAATAAACAAAAACGAAACCTCCATCTTTTTACATATAAAGATC

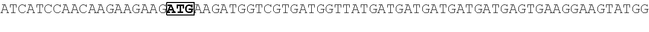

TAGATCAAACATGTAAACAGACACCAGACTTCAATCTCTGTGTCTCTCTACTCAACTCCGACCCACGTGGCTCTT
CTGCCGACACCTCTGGCCTCGCTCTCATCCTCATCGATAAAATCAAGGTATTTTTCAATTCCTTTTCTCATCTAG
TTTCTTCTATATAGATATTACCAATTATCTCAGATTATTTTCAAGTCTTATTATAAGAATCAAATCTTGACTAAA
GGTTTTGTGGTTGTTTTTTAAATTATGATATTTTTTCTATATTATTAGATGTAATATTTAATTTTATTCTATTCT
ATAACTTTGATCTCTTAAATTTTTATAAAAAGGCTCATAAGTTTCGTTATTCTACGAAAAAGTAATTATCACTAA
GACGTTTTTGTCTATAAGACTATAAGTAACACAAGGGGTTGTTTTTGATAAATAAGAAGTTTTTGATTACTTTTG
TTTAGAACACATACCTAAGCCTAAGGGTGTTATTTTTTTTTGTGTTTTCATGTCGTAGTAATATTGTTTTCAATT
TCAGTATAGTGTATATAAAGCTCGTTTGTCGTTTCTATCCCACCAATTATGTAGCTTTATTTTTCCAGAATTATC
TGAATTAAGGGGAGAGTTTAACTACAAATAAAAAATGTGAGGTAATTTCTGTTGAAATATAAACGTATGGGGTTA
TCTTATAAATTTTTTTTTGTAGGTTCTGGCGACAAAGACCTTAAACGAAATCAACGGTCTATATAAAAAGAGACC
GGAACTAAAACAGGCTTTAGACCAATGTAGTCGAAGATACAAAACGATCTTAAATGCTGATGTTCCCGAAGCCAT
CGAAGCTATCTCTAAAGGAGTCCCTAAATTTGGCGAAGATGGTGTGATCGACGCCGGGGTAGAAGCTTCTGTTTG
TGAAGAAGGGTTTCAAGGGAAATCTC

The total known sequence (promoter and coding sequence) of CWII2 from camelina is as follows with the start codon boxed.

SEQ ID NO: 110
TACGATGGACTCCAGAGCGGCCGCGGCGAGACGGTGAATGAACTAATGTGTATATATATGTATGACTT
ACTTTCGAATAATGAACTAATGTGTATGTATGACTTACTTTCGAATGAAGAAAGTTAGAAAGAATACA
AATTGATTCTTATTTCAGTTGTTCACATGTAAACACGTTATATGGCATCTTGACAAAAAGAAATATCA
CTTAATTCACATTGAGAATTCTTTTGTTTTCATATAGGACTATTATATATAGCAACAATATGTATCCT
GTAAATTTGAATCCCAATTGTAACAGCCATATATAATATTAGCATAACTATTGGACTAAATGTCATGG
TTAACGTAGTTAATGTGCTATTGTAATTAATTGTCATACCACGTAAAAATCAATAAAAGGTACTAAAA
TCATTTCATATTTTGCAACTACAAATGATAAACAAAAGTAGTATTTATTTTTATATATATTTTAAAAT
ACGTAATATCAAGAAACTGCTTAAAATATAAGACAAGAATCCTCTTTCTTCCATCTCTATCTCTCTCC

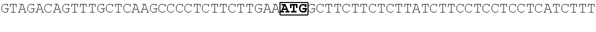

ACCCTATCCTTTCCATCCTCAACCCTAATCTCAGCCAAATCCAACGCGACAATAATCGAATCAACTTG
CAAAACCACGAACAACTACAAATTCTGTGTCTCGGCTCTCAAATCCGACCCAAGAAGTCCCACAGCCG
ACACAAAAGGTCTCGCAGCCATTATGATCGGCGTTGGTATGACAAACGCCACTTCCACCGCAACTTAC
ATCGCCGGAAACCTAACATCCGCTGCAAACGACGTCGTCCTTAAAAAGGTGTTACAAGATTGCTCCGA
GAAGTATGCTCTCGCCGCTGATTCTCTCCGTCAAACAATTCAATATCTTGATAATGAAGCTTATGACT
ATGCTTCCATGCATGTGCTGGCGGCGGAGGATTATCCTAATGTTTGCCGCAATATTTTCCGCCGAGCT
AAGGGGCTGTCTTATCCGGTGGAGATTCGTCGGCGTGAACAGAGTCTGAGACGTATCTGTGGTGTTGT
CTCAGGGATTCTTGATCGTCTTGTTGAA

These promoter sequences (SEQ ID NO:104 (cwII1); SEQ ID NO:105 (cwII2) can be used in fusion constructs with RNAi to cwII to inhibit cwII. Thus, for example, a fusion construct between the nucleotide sequences of SEQ ID NO:104 and SEQ ID NO:106 and/or between the nucleotide sequences of SEQ ID NO:105 and SEQ ID NO:107 can be constructed and used to inhibit cwII. Additionally, an RNAi construct of this invention for inhibition of cwII can include a fusion between the nucleotide sequences of SEQ ID NO:104 and SEQ ID NO:108 and/or between the nucleotide sequences of SEQ ID NO:105 and SEQ ID NO:108.

### Example 7

### Cloning of Single and Multi-Gene Expression Cassettes

The polynucleotides of interest (e.g., polynucleotides encoding polypeptides having the activity of succinyl-CoA synthetase, 2-oxoglutarate:ferredoxin oxidoreductase, 2-oxoglutarate, oxalosuccinate reductase and isocitrate lyase (i.e., the crTCA enzymes), glyoxylate carboligase, tartronic-semialdehyde reductase, superoxide reductase, a polynucleotide encoding an inhibitor of cwII, and/or a polynucleotide encoding an aquaporin) can be expressed singly or in polygene clusters as fusion proteins using the ubiquitin-based vector, or as linked, separate gene constructs within a T-DNA. In addition to over-expressing transgenes, we will have an RNAi construct made to suppress translation of endogenous cell wall invertase inhibitor (cwII). The transgenes will be in 4 clusters or links, and three crosses will be performed to obtain lines that will have all proposed transgenes expressed in single plant lines. These plant lines will then be evaluated for expression of the heterologous polynucleotides and for yield and performance.

### Example 8

### Transformation and Selection of Camelina

Camelina sativa variety (Ukraine) will be used and Agrobacterium-mediated transformation will be used for transformation. Camelina can be transformed by “floral dip” or vacuum application (Lu and Kang. Plant Cell Reports 27(2):273-278 (2008); Liu et al. In Vitro Cell Devel Biol-Animal. 44:S40-S41 (2008)) or any other method effective for the generation of stable camelina transformants. The Gateway vector with CaMV 35S promoter (Earley et al. Plant Journal. 45(4):616-629 (2006)) can be used for construction of the transgene cassettes. Gateway vectors or other vectors can be used for expression in seed, seed coat, or seed pod with the respective tissue specific promoter and/or targeting sequences.

To facilitate selection of seedlings after transformation of camelina, a selectable marker gene will be used together with a transgene. Thus, for each expression cassette, kanamycin, hygromycin B, bialaphos/ppt or DsRed selection (Lu and Kang. Plant Cell Reports 27(2):273-278 (2008)) can be used to facilitate selection of crossed seeds or seedlings between two clusters of genes. Double selection can be performed, followed by polymerase chain reaction (PCR) assays for each transgene to ensure the presence of the transgenes. Transgene expression can be monitored by Western and/or quantitative reverse transcriptase (qRT)-PCR, and validated by Northern blot analysis. Thus, four selectable markers will be used in selection from multiple crosses.

### Generating Homozygous Transgenic Lines

After “floral dip” transformation, about 1% of the seeds will be transgenic, and can be identified by selection. As discussed above, four different selectable marker genes will be evaluated: NPTII, HPT, BAR, and dsRed. After the selfing of the T1 plants, the seeds produced are the T2 generation. T2 plants should segregate to have ¼ homozygous for the transgene, ½ heterozygous for the transgene, and ¼ without transgene. Selection will be carried out on the T3 generation to identify homozygotes. The seeds of the lines from the T3 generation will be multiplied.

### Other Transgenic Plants

In some case, plants can be evaluated as heterozygotes. For plants from crosses, we will identify plants with desirable combinations of transgenes by double, triple or quadruple selection.

### Protocol for Transforming Camelina

Luria Broth (LB) medium for growing Agrobacterium
Infiltration medium:
½X MS salts
5% (w/v) Sucrose
0.044 uM BAP
0.05% Silwet L-77

### Procedure:

(1) Two days prior to transformation, a pre-culture of Agrobacterium carrying the appropriate binary vector is prepared by inoculating the Agrobacterium onto 3 ml LB medium including suitable antibiotics and incubating the culture at 28° C.
(2) One day prior to transformation a larger volume of (150 ml-300 ml) LB medium is inoculated with at least 1 ml of the preculture and incubated at 28° C. for about 16-24 hrs.
(3) Water plants prior to transformation.
(4) On the day of transformation of the plant, Agrobacterium cells are pelleted by centrifugation at 6000 rpm for 10 min at room temperature (e.g., about 19° C. to about 24° C.).
(5) The pellet is resuspended in 300-600 ml of infiltration medium (note: the infiltration medium is about double the volume used in the agro culture (about 150-300 ml)).
(6) The suspension solution is transferred to an open container that can hold the volume of infiltration medium prepared (300-600 ml) in which plants can be dipped and which fits into a desiccator.
(7) Place the container from (6) into a desiccator, invert a plant and dip the inflorescence shoots into the infiltration medium.
(8) Connect the desiccator to a vacuum pump and evacuate for 5 min at 16-85 kPa.
(9) Release the vacuum slowly.
(10) After releasing vacuum, remove the plants and orient them into an upright position or on their sides in a plastic nursery flat, and place a cover over them for the next 24 hours.
(11) The next day, the cover is removed, the plants rinsed with water and returned to their normal growing conditions (e.g., of about 22° C./18° C. (day/night) with daily watering under about 250-400 μE white light).
(12) A week later the plants were transformed again, repeating steps 1-11.
(13) The plants were watered on alternate days beginning after transformation for about 2-3 weeks and then twice a week for about another 2 weeks after which they were watered about once a week for about another 2-3 weeks for drying.

### Example 8

### Analysis of Transformed C. sativa Plants

### (1) Verification of Expression in the Various Plant Organelles

RT-PCR and pRT-PCR Methods.

RNA is isolated using the RNeasy kit (Qiagen), with an additional DNase I treatment to remove contaminating genomic DNA. Reverse transcription (RT) was carried out to generate cDNA using Omniscript reverse transcriptase enzyme (Qiagen). GFP-fused-SOR transcripts can be detected by PCR as described by Im et al., (2005) using internal GFP forward and gene specific primers (SOR reverse and actin specific primers), APX specific primers described in (Panchuk et al. Plant Physiol 129: 838-853 (2002) and Zat12 specific primers (forward; 5′ AACACAAACCACAAGAGGATCA 3′ (SEQ ID NO:111) and reverse; 5′ CGTCAACGTTTTCTTGTCCA 3′ (SEQ ID NO:112)). Quantitative RT-PCR was carried out using Full Velocity SYBR-Green® QPCR Master Mix (Stratagene) on a MX3000P thermocycler (Stratagene). Gene specific primers for select genes were designed with the help of AtRTPrimer, a database for generating specific RT-PCR primer pairs (Han and Kim, BMC Bioinformatics 7:179 (2006)). Relative gene expression data were generated using the 2^{-ΔΔCt} method (Livak and Schmittgen, Methods 25:402-408 (2001)) using the wild-type zero time point as the reference. PCR conditions were 1 cycle of 95° C. for 10 min, 95° C. for 15 s, and 60° C. for 30 s to see the dissociation curve, 40 cycles of 95° C. for 1 minute for DNA denaturation, and 55° C. for 30 s for DNA annealing and extension.

### Immunoblotting (Western Analysis for SOR Detection)

Total protein extract is obtained from liquid N₂ frozen plants or seedlings grown as described by Weigel and Glazebrook, Arabidopsis: A Laboratory Manual. Cold Spring Harbor Laboratory Press, Cold Spring Harbor, N.Y. (2002)). Protein concentration is quantified as described by Bradford (Anal Biochem 72: 248-254, (1976)). Protein is separated by 10% (w/v) SDS-PAGE and detected with rabbit antibodies raised against P. furiosus SOR (at 1:2,000 dilution) or antibodies raised against HSP70, BiP, and CRT (at 1:1,000 dilution). Immunoreactivity is visualized with either horseradish peroxidase-conjugated anti-rabbit or anti-mouse antibodies (Pierce, Rockford, Ill.).

### SOR Activity Assay

Samples are ground with liquid nitrogen and lysed as described previously (Im et al., FEBS Lett 579: 5521-5526 (2005)). Samples are centrifuged at 27,000 g at 4° C. for 30 min and resulting supernatants are passed through a 0.45 micron filter unit to remove cellular debris. Extracts are dialyzed overnight in 50 mM phosphate buffer. To reduce plant SOD background activity of dialyzed samples, samples are heat-treated (heat-treated at 80° C. for 15 min) and centrifuged at 21,000 g for 15 min. The heat treatments used are sufficient to inactivate some endogenous plant SOD activity, allowing for greater discrimination between SOD and SOR activity in the transgenic plants. To avoid leaf pigments and reduce loss of activity resulting from dialysis, roots are harvested from seedlings grown for 28 days or 42 days on agar plates in a growth chamber (8 h light/16 h dark).

The standard SOD/SOR assay is performed as described in Im et al. (FEBS Lett 579: 5521-5526 (2005)). One unit of SOD/SOR activity is defined as the amount of enzyme that inhibits the rate of reduction of cytochrome c by 50% (McCord and Fridovich, J Biol Chem 244: 6049-6055 (1969)).

### (2) Reduction in ROS

### H₂O₂ Measurements (FOX Assay)

A ferrous ammonium sulfate/xylenol orange (FOX) method is used to quantify H₂O₂ in plant extracts (Wolff, Methods Enzymol 233: 182-189, 1994)). The original FOX method is modified by addition of an acidification step where 1 ml of 25 mM H₂SO₄ was added to each sample to allow for precipitation of interfering substances (sugars, starches, polysaccharides) for 15 min on ice, and centrifuged at 9,700 g, for 15 min, at 4° C. The cell free extract is collected and passed through a 0.45 □m-filter unit. 100 μl is added to 1 ml of the FOX reagent, mixed, and incubated at room temperature for 20 min. The concentration of H₂O₂ in the reagent is calibrated using absorbance at 240 nm and an extinction coefficient of 43.6 M⁻¹ cm⁻¹. The concentration of H₂O₂ is measured in nmoles H₂O₂ per gram of fresh wt cells.

### Ascorbate Peroxidase (APX) Activity Assay

APX activity is determined as described previously (Nakano and Asada, Plant Cell Physiol 22:867-880, 1981). Fifty μg of the extract is used in a 3 ml APX assay and the reaction proceeds for 2 minutes. APX activity is expressed as μmol of ascorbate oxidized (mg protein)⁻¹ min⁻¹. Additional confirmation of APX activity can be done by an in-gel assay as described by Panchuk et al. (Plant Physiol 129: 838-853 (2002)).

### (3) Protection of the Photosynthetic Apparatus and its Surrounding Membrane Lipids

To quantify the protection of the photosystems, leaf fluorescence and CO2 fixation rates of fully expanded leaves is measured using a LiCOR system. The maximal photochemical efficiency of the PSII is calculated using the ratio Fᵥ/Fₘ, where Fᵥ=Fₘ−Fₒ (Genty et al., Biochimica et Biophysica Acta (BBA)—General Subjects 990: 87-92 (1989)). This is calculated from initial (Fₒ) and maximum fluorescence (Fₘ) as measured in vivo on the last fully expanded leaf pre-acclimatized to the dark for approximately 40 min. Fₘ can be estimated by applying a light saturating flash with an intensity of ca. 8,000 μmol photons m⁻²s⁻¹.

### (4) Reduction in Photorespiration

Reduction in photorespiration is determined by CO₂ fixation rates as described above using a LICOR system. Plants are exposed to atmospheric CO₂:O₂ mixtures (400 ppm CO₂/21% O₂) or at saturating CO₂ concentrations (4000 ppm/21% O₂) and their biomass, photosynthetic CO₂ fixation rates, chlorophyll fluorescence and chlorophyll content are quantified. Higher CO₂ fixation rates in the transgenic plants under limiting CO₂ compared to wild type and control plants indicate reduced photorespiratory activity.

### (5) Increased Tolerance to Abiotic Stress

### Thermotolerance Assays

To test seed basal thermotolerance, stratified seeds are treated at 45° C. for 5 h and germination was evaluated 2 days (d) later following the protocol of Larkindale et al. Plant Physiol 138: 882-897 (2005), The hypocotyl elongation assay was carried out as described by Hong and Vierling, (Proc Natl Acad Sci USA 97: 4392-4397 (2000)). Growth after the heat treatment was measured and compared with that of seedlings receiving no heat treatment. For tests of vegetative-stage plants, 10 day-old grown seedlings were used as described by Hong and Vierling (Proc Natl Acad Sci USA 97: 4392-4397 (2000)). Heat-treated plates were returned to the 22° C. incubator and all plates were left at 22° C. for 7 d. The number of seedlings that survived were counted after 7 d.

Mature, flowering plants grown at 22° C. are exposed for 0 days, 2 days, 4 days, 6 days and 10 days to 35° C. Survival rate, seed set, flower number, chlorophyll content and total final seed number, seed weight and seed germination rate is analyzed per plant.

### Quantification of Chlorophyll for Plants Exposed to Heat Challenge

Etiolated seedlings were grown for 2.5 days in the dark at 22° C.; exposed to 48° C. for 30 min in the dark, and transferred to continuous light for 24 hrs. Seedlings were ground with liquid nitrogen and extracted with 80% (v/v) acetone by shaking until the leaves became bleached. The chlorophyll content in the acetone extract was quantified spectrophotometrically based on absorbance at 663 nm as described by (Burke et al. Plant Physiol. 123:575-588 (2000)).

### SOR Protection Against Chemically Induced ROS

Seeds (25 seeds of each line) are sterilized and plated on a single plate of 0.8% MS medium containing different concentrations of paraquat (0, 0.25, 0.5 and 1 μM). Plant survival (number of green seedlings) is calculated for each line after 14 d under continuous light. Results are reported as percent of each control (100%) and show mean±SD from 3 independent experiments.

### (6) Reduction in Lignin Polymerization

### Histochemical Staining

In order to examine the lignified cell walls in stems, the transgenic and WT plants are grown under the same conditions for 2 months. The second internodes of stems (from ground level) are excised, the bark removed, and the internodes hand-cut into 20-30 μm thick slices, and subjected to histochemical analysis. Wiesner staining is performed by incubating sections in 1% phloroglucinol (w/v) in 6 mol l⁻¹ HCl for 5 min, and the sections observed under a dissecting microscope (Pomar et al., Protoplasma 220:17-28 (2002); Weng et al., The Plant Cell 22, 1033-1045 (2010). For Mäule staining, hand-cut stem sections are soaked in 1% KMnO₄ for 5 min, then rinsed with water, destained in 30% HCl, washed with water, mounted in concentrated NH₄OH, and examined under a dissecting microscope (Atanassova et al., The Plant Journal 8, 465-477 (1995); Weng et al., The Plant Cell 22, 1033-1045 (2010)).

### Assay of Klason Lignin Content

The second internodes of stems (from ground level) of transgenic and WT plants grown under the same conditions for approximately 2 months, are excised, the bark removed, and the internodes then cut into thin sections and put into an 80° C. oven. The dried stem materials are ground into a fine powder, extracted four times in methanol and dried. Then 200 mg of the extract is mixed with 5 ml of 72% (w/w) sulfuric acid at 30° C. and hydrolyzed for 1 h. The hydrolysate was diluted to 4% sulfur by the addition of water and then cooked for 1 h in boiling water. The solid residue is filtered through a glass filter. Finally, the sample is washed, dried at 80° C. overnight and then weighed. The lignin content is measured and expressed as a percentage of the original weight of cell wall residue (Dence C. 1992. Lignin determination. In: Lin S, ed., Methods in lignin chemistry. Berlin: Springer-Verlag, 33-61).

### (7) Increased Accessibility to Cell Wall Cellulose by an Enzyme

### Cellulose Accessibility

The cellulose accessibility of biomass and the pure cellulose samples is determined using fluorescence-labeled, purified Trichoderma reesei Cel7A. Triplicate samples (250 mL final volume) containing 1.0 mM T. reesei Cel7A with a substrate concentration equivalent to 1.0 mg mL⁻¹ final cellulose concentration in 5 mM sodium acetate pH 5.0 buffer are prepared for each reaction time assayed throughout a 120 h time course. Reactions are conducted at 38° C., rotating end-over-end and assayed at 1, 4, 24, 48, and 120 h. Each reaction is initiated by the addition of enzyme and terminated by filtration in a 96-well vacuum filter manifold (Innovative Microplate, Chicopee, Mass.) equipped with a 1.0 mm glass fiber filter. The reaction supernatant is assayed for reducing sugars using the BCA method (Doner and Irwin, Anal Biochem 202(1):50-531992) against a cellobiose standard curve. The solid fraction retained in the filter was assayed for bound T. reesei Cel7A concentration.

### Bound Cellulase Enzyme Quantitation

The concentration of bound enzyme on the solids fraction from the accessibility experiments is assayed by fluorometry with adjustments for biomass autofluorescence. Following filtration of the reaction samples, the retained solids (containing pure cellulose samples (PCS) bound T. reesei Cel7A) are resuspended with 250 mL of distilled water. For each sample, 150 mL of the resuspended solids are transferred to a microtiter plate and read in a FLUOstar optima plate reader (BMG Labtechnologies, Durham, N.C.) at excitation and emission wavelengths of 584 and 612 nm, respectively. The emission intensities from the samples are converted to concentrations of T. reesei Cel7A using regression parameters from a standard curve of calibration standards that are measured concurrently. To negate the autofluorescence of each of the PCS, a separate calibration is made for each PCS sample digested with Cel7A. The calibration curves contain six levels of standard additions (0-1 mM T. reesei Cel7A) with the same concentration of PCS as used in each of the accessibility experiments. To negate the effects of plate-to-plate or day-to-day variations in the fluorescence measurements, a fresh set of calibration standards (in triplicate, with the appropriate PCS sample) is included with each microtiter plate containing unknown samples from the reactions.

The effect of digestion on the correction of autofluorescence in the calibration standards is examined as follows. Fifteen replicates of a PCS sample are digested to 67±9% by unlabeled T. reesei Cel7A in 5-days, using the conditions described above for the cellulase accessibility experiments. The reactions are terminated by filtration and the solids fractions re-suspended in 125 mL of distilled water. The re-suspended solids are transferred to a microtiter plate, with 75 mL from each replicate pipetted into each well. Standard additions of fluorescence-labeled T. reesei Cel7A including five levels ranging from 0.12 to 2 mM are prepared. Each amount is pipetted in triplicate (75 mL per replicate) to the wells containing digested PCS. Calibration standards with the same final T. reesei concentrations are then prepared in the same microtiter plate, using undigested PCS. The plate is read in the fluorometer as described earlier. The concentrations of T. reesei Cel7A with the digested PCS are determined using regression parameters from the standard curve developed using the undigested PCS. These values are compared to the expected values to determine the effect of extensive digestion on the quantitation method.

Methods for the pQE-1 crTCA enzyme expression constructs are provided in Example 1. A standard calcium chloride transformation method is employed for transforming E. coli.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

1. A method for increasing carbon fixation and/or increasing biomass production in a plant, comprising:
introducing into a plant, plant part, and/or plant cell one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase and (e) isocitrate lyase to produce a stably transformed plant, plant part, and/or plant cell expressing said one or more heterologous polynucleotides.

2. The method of claim 1, wherein the one or more heterologous polynucleotides are introduced into a nucleus and/or a chloroplast of said plant, plant part, and/or plant cell.

3. The method of claim 1, wherein one or more of said polypeptides are operably linked to an amino acid sequence that targets said polypeptides to the chloroplast.

4. The method of claim 1, wherein the succinyl CoA synthetase is from Escherichia coli, Azotobacter vinelandii, Bradyrhizobium sp., Azospirillum sp., or any combination thereof; the 2-oxoglutarate:ferredoxin oxidoreductase is from Paenibacillus sp., Halobacterium sp., Hydrogenobacter thermophilus, Bacillus sp, Paenibacillus larvae subsp. larvae, Haladaptus paucihalophilus, Magnetococcus sp., or any combination thereof; the 2-oxoglutarate carboxylase is from Candidatus Nitrospira defluvii, Hydrogenobacter thermophilus, Thiocystis violascens, Mariprofundus ferroxydans, Pseudomonas stutzeri, or any combination thereof; the oxalosuccinate reductase is from Acinetobacter baumannii, Chlorobium limicola, Kosmotoga olearia, Marine gamma proteobacterium, or any combination thereof; and/or the isocitrate lyase is from Corynebacterium glutamicum, Gordonia alkanivorans, Nocardia farcinica, Rhodococcus pyridinivorans, Rhodococcus jostii, or any combination thereof.

5. The method of claim 1, further comprising
introducing into the plant, plant part, and/or plant cell one or more heterologous polynucleotides encoding polypeptides having the enzyme activity of glyoxylate carboligase and tartronic semialdehyde reductase to produce a stably transformed plant, plant part, and/or plant cell expressing said one or more heterologous polynucleotides.

6. The method of claim 1, further comprising
introducing into the plant, plant part, and/or plant cell a heterologous polynucleotide encoding a superoxide reductase from an archaeon species, a heterologous polynucleotide encoding an CO₂ transporter, a heterologous polynucleotide encoding an RNAi inhibitor of cell wall invertase inhibitor, or any combination thereof, to produce a stably transformed plant, plant part, and/or plant cell expressing said heterologous polynucleotide(s).

8. A stably transformed plant, plant part or plant cell produced by the method of claim 1.

9. A stably transformed plant, plant part or plant cell comprising one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase.

10. The stably transformed plant, plant part or plant cell of claim 9, wherein the succinyl CoA synthetase is from Escherichia coli, Azotobacter vinelandii DJ, Bradyrhizobium sp., Azospirillum sp., or any combination thereof; the 2-oxoglutarate:ferredoxin oxidoreductase is from Paenibacillus sp., Halobacterium sp, Hydrogenobacter thermophilus, Bacillus sp, Paenibacillus larvae subsp. larvae, Haladaptus paucihalophilus, Magnetococcus sp., or any combination thereof; the 2-oxoglutarate carboxylase is from Candidatus Nitrospira defluvii, Hydrogenobacter thermophilus, Candidatus Nitrospira defluvii, Hydrogenobacter thermophilus, Thiocystis violascens, Mariprofundus ferroxydans, Pseudomonas stutzeri, or any combination thereof; the oxalosuccinate reductase is from Acinetobacter baumannii, Chlorobium limicola, Kosmotoga olearia, marine gamma proteobacterium, or any combination thereof; and/or the isocitrate lyase is from the Corynebacterium glutamicum, Gordonia alkanivorans, Nocardia farcinica, Rhodococcus pyridinivorans, Rhodococcus jostii, or any combination thereof.

11. The stably transformed plant, plant part or plant cell of claim 9, further comprising one or more heterologous polynucleotides encoding polypeptides having the enzyme activity of glyoxylate carboligase and tartronic semialdehyde reductase to produce a stably transformed plant, plant part, and/or plant cell expressing said one or more heterologous polynucleotides.

12. The stably transformed plant, plant part or plant cell of claim 9, further comprising a heterologous polynucleotide encoding a superoxide reductase from an archaeon species, a heterologous polynucleotide encoding CO₂ transporter, a heterologous polynucleotide encoding an RNAi inhibitor of cell wall invertase inhibitor, or any combination thereof.

13. A seed of the stably transformed plant of claim 9, wherein the seed comprises in its genome the one or more heterologous polynucleotides encoding polypeptides having the enzyme activity of (a) succinyl CoA synthetase, (b) 2-oxoglutarate:ferredoxin oxidoreductase, (c) 2-oxoglutarate carboxylase, (d) oxalosuccinate reductase, and (e) isocitrate lyase.

14. A product produced from the stably transformed plant, plant part or plant cell of claim 9.

15. A product produced from the stably transformed plant, plant part or plant cell of claim 11.

16. A product produced from the stably transformed plant, plant part or plant cell of claim 12.

17. A product produced from the stably transformed seed of claim 13.

19. The product of claim 14, wherein the product is a food, drink, animal feed, fiber, oil, pharmaceutical and/or biofuel.

19. The product of claim 15, wherein the product is a food, drink, animal feed, fiber, oil, pharmaceutical and/or biofuel.

20. The product of claim 17, wherein the product is a food, drink, animal feed, fiber, oil, pharmaceutical and/or biofuel.
